(19) United States
(12) Patent Application Publication
(10) Pub. No.: US 2025/0250265 A1
(43) Pub. Date: Aug. 7, 2025

(54) BENZOTHIAZOLE-PHENYLSULFONYL-PIPERIDINE ANALOGS AS ACTIVATORS OF NACYLPHOSPHATIDYLETHANOLAMINE HYDROLYZING PHOSPHOLIPASE D

(71) Applicant: Vanderbilt University, Nashville, TN (US)

(72) Inventors: Sean Davies, Nashville, TN (US); Gary A. Sulikowski, Nashville, TN (US); Kwangho Kim, Nashville, TN (US); Alex G. Waterson, Nashville, TN (US); Ian M. Romaine, Nashville, TN (US); Paige N. Vinson, Nashville, TN (US); Charles David Weaver, Nashville, TN (US); Jonah Elliot Zarrow, Encino, CA (US); Geetika Aggarwal, Saint Louis, MO (US); Rocco D. Gogliotti, Kingston Springs, TN (US)

(21) Appl. No.: 18/855,876

(22) PCT Filed: Apr. 14, 2023
(86) PCT No.: PCT/US2023/018597

§ 371 Date: Oct. 10, 2024

Related U.S. Application Data
(60) Provisional application No. 63/331,322, filed on Apr. 15, 2022.

(51) Int. Cl. C07D 417/12; A61K 31/454; A61K 31/4545; A61P 1/16; A61P 3/10; C07D 417/14

(52) U.S. Cl. CPC C07D 417/12; A61K 31/454; A61K 31/4545; A61P 1/16; A61P 3/10; C07D 417/14

(57) ABSTRACT

Described herein are benzothiazole-phenyl-sulfonyl-piperidine compounds for activating N-acyl-phosphatidylethanolamine hydrolyzing phospholipase D (NAPE-PLD).

### CROSS-REFERENCE TO RELATED APPLICATIONS

This application claims priority to U.S. Provisional Patent Application No. 63/331,322, filed on Apr. 15, 2022, which is incorporated by reference herein in its entirety.

### TECHNICAL FIELD

Described herein are benzothiazole-phenylsulfonyl-piperidine (BT-PSP) compounds for activating N-acyl-phosphatidylethanolamine hydrolyzing phospholipase D (NAPE-PLD).

### BACKGROUND

Non-endocannabinoid N-acyl-ethanolamides (NAEs), including N-oleoyl-ethanolamide (OEA) and N-palmitoylethanolamide (PEA), are bioactive lipids that exert pleiotropic protective effects against metabolic diseases. OEA is rapidly biosynthesized in the intestinal tract in response to food intake and promotes satiety, fatty acid oxidation, and glucose-stimulated insulin secretion. Administering OEA to rodents fed a high-fat diet reduces food intake, fat accumulation, hyperglycemia, hyperlipidemia, inflammation, and hepatic steatosis. Like OEA, PEA is also biosynthesized in many peripheral tissues and exerts significant anti-inflammatory effects including inhibiting leukocyte chemotaxis to inflammatory stimuli and mast cell activation, and enhances the efferocytotic capacity of macrophages, which is essential for the resolution of inflammation. Administering PEA to rodents inhibits inflammation induced by various stimuli and reduces hypertriglyceridemia, and atherosclerotic lesion area and necrosis in atherosclerosis-prone mice fed a Western Diet.

However, although repeated i.p. injection of OEA or PEA or their precursors significantly blunts metabolic disease in rodents, delivering them in adequate doses in humans has been challenging due to their poor solubility and rapid metabolism. Therefore, the early promise of administering OEA, PEA, and their precursors has failed to translate adequately in human trials.

N-acyl-phosphatidylethanolamine hydrolyzing phospholipase D (NAPE-PLD) is a beta-lactamase fold zinc metallohydrolase that catalyzes OEA and PEA biosynthesis by hydrolyzing appropriate precursor N-acyl-phosphatidylethanolamines (NAPEs). Increasing intestinal NAPE-PLD expression via an adenoviral vector increased intestinal OEA and PEA levels and reduced food intake compared to the control vector. High-fat diets markedly reduce NAPE-PLD expression and OEA and PEA levels in many tissues including intestine, aorta, spleen, and bone marrow. Mimicking this by selective deletion of intestinal NAPE-PLD reduced intestinal OEA and PEA levels and increased adiposity, hypertriglyceridemia, and hepatic steatosis. Selective deletion of hepatocyte NAPE-PLD resulted in hepatic steatosis and increased body fat. Selective deletion of adipocyte NAPE-PLD increased adiposity and hyperglycemia and prevented cold-induced adipocyte browning.

Given the metabolic defects induced by genetic deletion of NAPE-PLD, the effect of high-fat diets on NAPE-PLD expression, and the metabolic benefits from elevating OEA and PEA levels, small molecule activators of NAPE-PLD should restore normal levels of OEA and PEA in vivo and markedly reduce manifestations of metabolic disease induced by high-fat/high-fructose diets including adiposity, hyperglycemia, hyperlipidemia, insulin resistance, inflammation, and poor wound healing.

What is needed are small molecule activators of NAPE-PLD that will induce increased NAE levels in vivo.

### SUMMARY

One embodiment described herein is a compound of formula (I):

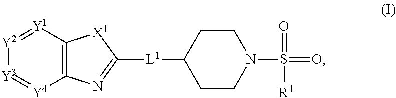

or a pharmaceutically acceptable salt thereof,
wherein:
X¹ is S or O;
Y¹ and Y³ are each C—R² and Y² and Y⁴ are each C—H, or
Y¹ and Y³ are each C—H and Y² and Y⁴ are each C—R²;
L¹ is

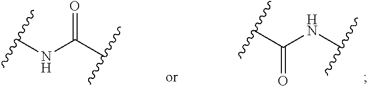 [SMILES: *NC(*)=O ; *NC(*)=O]

R¹ is G¹, —CH₂-G¹, or C₁₋₄alkyl;
R², at each occurrence, is independently C₁₋₄alkyl, C₁₋₄haloalkyl, halogen, C₃₋₄cycloalkyl, —OC₁₋₄alkyl, —OC₁₋₂haloalkyl, or —OC₃₋₄cycloalkyl;
G¹ is a phenyl, a 5- to 9-membered heteroaryl containing 1-3 heteroatoms, a 3- to 7-membered carbocycle, or a 4- to 6-membered heterocycle containing 1-2 heteroatoms, wherein G¹ is optionally substituted with 1 to 3 substituents, each independently R¹¹ or —C₁₋₂alkylene-R¹¹;
R¹¹ is C₁₋₄alkyl, C₁₋₄haloalkyl, C₃₋₄cycloalkyl, halogen, cyano, —OR^{11a}, —N(R^{11a})₂, —NR^{11a}C(O)R^{11b}, —C(O) R^{11b}, —CO₂R^{11b}, or —SO₂R^{11b};
R^{11a}, at each occurrence, are each independently hydrogen, C₁₋₄alkyl, C₁₋₄haloalkyl, or C₃₋₄cycloalkyl; and
R^{11b}, at each occurrence, is independently C₁₋₄alkyl, C₁₋₄haloalkyl, or C₃₋₄cycloalkyl.

In one aspect, X¹ is S.

In another aspect Y¹ and Y³ are each C—C₁₋₄alkyl and Y² and Y⁴ are each C—H, or, where Y¹ and Y³ are each C—H and Y² and Y⁴ are each C—C₁₋₄alkyl.

In another aspect, Y¹ and Y³ are each C—CH₃, and Y² and Y⁴ are each C—H.

In another aspect, Y¹ and Y³ are each C—H, and Y² and Y⁴ are each C—CH₃.

In another aspect, L¹ is

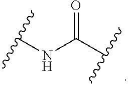 [SMILES: *NC(*)=O]

In another aspect, R¹ is G¹.

In another aspect, G¹ is the optionally substituted phenyl.

In another aspect, G¹ is

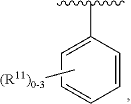

wherein R¹¹, at each occurrence, is independently halogen, cyano, C₁₋₄alkyl, C₁₋₂haloalkyl, —OC₁₋₄alkyl, or —OC₁₋₂haloalkyl.

In another aspect, G¹ is

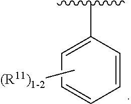

In another aspect, G¹ is

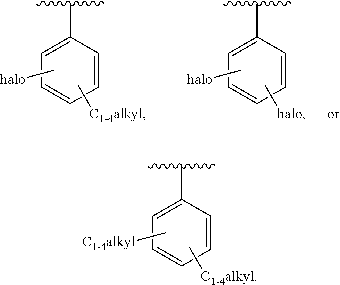

In another aspect, G¹ is

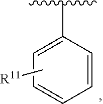

where R¹¹ is halogen, cyano, C₁₋₄alkyl, C₁₋₂haloalkyl, —OC₁₋₄alkyl, or —OC₁₋₂haloalkyl.

In another aspect, G¹ is the optionally substituted 5- to 9-membered heteroaryl.

In another aspect, the ring system of the optionally substituted 5- to 9-membered heteroaryl is a pyridinyl, a pyrimidinyl, a pyrazolyl, or a benzo[c][1,2,5]oxadiazolyl.

In another aspect, R¹ is

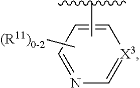

wherein X³ is C—H, or N, and R¹¹, at each occurrence, is independently halogen, cyano, C₁₋₄alkyl, C₁₋₂haloalkyl, —OC₁₋₄alkyl, or —OC₁₋₂haloalkyl.

In another aspect, R¹ is

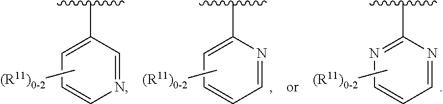

In another aspect, R¹ is

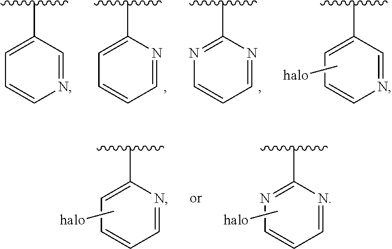

In another aspect, R¹ is —CH₂-G¹.

In another aspect, G¹ is the optionally substituted 3- to 7-membered carbocycle.

In another aspect, the ring system of the optionally substituted 3- to 7-membered carbocycle is a 3- or 6-membered carbocycle.

In another aspect, R¹ is

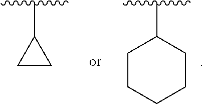

In another aspect, R¹ is C₁₋₄alkyl.

In another aspect, R¹ is methyl.

In another aspect, the compound is
1-((3-chloro-4-fluorophenyl)sulfonyl)-N-(5,7-dimethylbenzo[d]thiazol-2-yl)piperidine-4-carboxamide;
N-(4,6-dimethylbenzo[d]thiazol-2-yl)-1-((4-fluoro-2-methylphenyl)sulfonyl)piperidine-4-carboxamide;
N-(4,6-dimethylbenzo[d]thiazol-2-yl)-1-(pyridin-3-ylsulfonyl)piperidine-4-carboxamide;
N-(4,6-dimethylbenzo[d]thiazol-2-yl)-1-((1-methyl-1H-pyrazol-4-yl)sulfonyl)piperidine-4-carboxamide;
1-((2-chlorophenyl)sulfonyl)-N-(4,6-dimethylbenzo[d]thiazol-2-yl)piperidine-4-carboxamide;
N-(4,6-dimethylbenzo[d]thiazol-2-yl)-1-(o-tolylsulfonyl)piperidine-4-carboxamide;
N-(4,6-dimethylbenzo[d]thiazol-2-yl)-1-((3-fluorophenyl)sulfonyl)piperidine-4-carboxamide;
1-((3-chlorophenyl)sulfonyl)-N-(4,6-dimethylbenzo[d]thiazol-2-yl)piperidine-4-carboxamide;
N-(4,6-dimethylbenzo[d]thiazol-2-yl)-1-(m-tolylsulfonyl)piperidine-4-carboxamide;
1-((4-cyanophenyl)sulfonyl)-N-(4,6-dimethylbenzo[d]thiazol-2-yl)piperidine-4-carboxamide;
N-(4,6-dimethylbenzo[d]thiazol-2-yl)-1-((4-(trifluoromethoxy)phenyl)sulfonyl)piperidine-4-carboxamide;
1-((2-chloro-4-fluorophenyl)sulfonyl)-N-(4,6-dimethylbenzo[d]thiazol-2-yl)piperidine-4-carboxamide;
N-(4,6-dimethylbenzo[d]thiazol-2-yl)-1-((4-fluoro-2-methylphenyl)sulfonyl)piperidine-4-carboxamide;
N-(4,6-dimethylbenzo[d]thiazol-2-yl)-1-((6-fluoropyridin-3-yl)sulfonyl)piperidine-4-carboxamide;
N-(4,6-dimethylbenzo[d]thiazol-2-yl)-1-((5-fluoropyridin-2-yl)sulfonyl)piperidine-4-carboxamide;
N-(4,6-dimethylbenzo[d]thiazol-2-yl)-1-((5-fluoropyrimidin-2-yl)sulfonyl)piperidine-4-carboxamide;
N-(4,6-dimethylbenzo[d]thiazol-2-yl)-1-((2-fluoropyridin-3-yl)sulfonyl)piperidine-4-carboxamide;
N-(4,6-dimethylbenzo[d]thiazol-2-yl)-1-((3-fluoropyridin-2-yl)sulfonyl)piperidine-4-carboxamide;
N-(4,6-dimethylbenzo[d]thiazol-2-yl)-1-((3-fluoro-4-methylphenyl)sulfonyl)piperidine-4-carboxamide;
1-(cyclohexylsulfonyl)-N-(4,6-dimethylbenzo[d]thiazol-2-yl)piperidine-4-carboxamide;
1-((3,4-dichlorophenyl)sulfonyl)-N-(4,6-dimethylbenzo[d]thiazol-2-yl)piperidine-4-carboxamide;
1-((2,4-difluorobenzyl)sulfonyl)-N-(4,6-dimethylbenzo[d]thiazol-2-yl)piperidine-4-carboxamide;
1-(cyclopropylsulfonyl)-N-(4,6-dimethylbenzo[d]thiazol-2-yl)piperidine-4-carboxamide;
1-((cyclopropylmethyl)sulfonyl)-N-(4,6-dimethylbenzo[d]thiazol-2-yl)piperidine-4-carboxamide;
1-(benzo[c][1,2,5]oxadiazol-4-ylsulfonyl)-N-(4,6-dimethylbenzo[d]thiazol-2-yl)piperidine-4-carboxamide;
1-((3-chloro-4-fluorophenyl)sulfonyl)-N-(5,7-dimethylbenzo[d]thiazol-2-yl)piperidine-4-carboxamide;
N-(5,7-dimethylbenzo[d]thiazol-2-yl)-1-((4-fluoro-2-methylphenyl)sulfonyl)piperidine-4-carboxamide;
N-(5,7-dimethylbenzo[d]thiazol-2-yl)-1-(pyridin-3-ylsulfonyl)piperidine-4-carboxamide;
N-(5,7-dimethylbenzo[d]thiazol-2-yl)-1-((1-methyl-1H-pyrazol-4-yl)sulfonyl)piperidine-4-carboxamide;
1-((2-chlorophenyl)sulfonyl)-N-(5,7-dimethylbenzo[d]thiazol-2-yl)piperidine-4-carboxamide;
N-(5,7-dimethylbenzo[d]thiazol-2-yl)-1-(o-tolylsulfonyl)piperidine-4-carboxamide;
N-(5,7-dimethylbenzo[d]thiazol-2-yl)-1-((3-fluorophenyl)sulfonyl)piperidine-4-carboxamide;
1-((3-chlorophenyl)sulfonyl)-N-(5,7-dimethylbenzo[d]thiazol-2-yl)piperidine-4-carboxamide;
N-(5,7-dimethylbenzo[d]thiazol-2-yl)-1-(m-tolylsulfonyl)piperidine-4-carboxamide;
1-((4-cyanophenyl)sulfonyl)-N-(5,7-dimethylbenzo[d]thiazol-2-yl)piperidine-4-carboxamide;
N-(5,7-dimethylbenzo[d]thiazol-2-yl)-1-((4-(trifluoromethoxy)phenyl)sulfonyl)piperidine-4-carboxamide;
1-((2-chloro-4-fluorophenyl)sulfonyl)-N-(5,7-dimethylbenzo[d]thiazol-2-yl)piperidine-4-carboxamide;
N-(5,7-dimethylbenzo[d]thiazol-2-yl)-1-((4-fluoro-2-methylphenyl)sulfonyl)piperidine-4-carboxamide;
N-(5,7-dimethylbenzo[d]thiazol-2-yl)-1-((6-fluoropyridin-3-yl)sulfonyl)piperidine-4-carboxamide;
N-(5,7-dimethylbenzo[d]thiazol-2-yl)-1-((5-fluoropyridin-2-yl)sulfonyl)piperidine-4-carboxamide;
N-(5,7-dimethylbenzo[d]thiazol-2-yl)-1-((5-fluoropyrimidin-2-yl)sulfonyl)piperidine-4-carboxamide;
N-(5,7-dimethylbenzo[d]thiazol-2-yl)-1-((2-fluoropyridin-3-yl)sulfonyl)piperidine-4-carboxamide;
N-(5,7-dimethylbenzo[d]thiazol-2-yl)-1-((3-fluoropyridin-2-yl)sulfonyl)piperidine-4-carboxamide;
N-(5,7-dimethylbenzo[d]thiazol-2-yl)-1-((3-fluoro-4-methylphenyl)sulfonyl)piperidine-4-carboxamide;
1-(cyclohexylsulfonyl)-N-(5,7-dimethylbenzo[d]thiazol-2-yl)piperidine-4-carboxamide;
1-((3,4-dichlorophenyl)sulfonyl)-N-(5,7-dimethylbenzo[d]thiazol-2-yl)piperidine-4-carboxamide;
1-((2,4-difluorobenzyl)sulfonyl)-N-(5,7-dimethylbenzo[d]thiazol-2-yl)piperidine-4-carboxamide;
1-(cyclopropylsulfonyl)-N-(5,7-dimethylbenzo[d]thiazol-2-yl)piperidine-4-carboxamide;
1-((cyclopropylmethyl)sulfonyl)-N-(5,7-dimethylbenzo[d]thiazol-2-yl)piperidine-4-carboxamide; or
1-(benzo[c][1,2,5]oxadiazol-4-ylsulfonyl)-N-(5,7-dimethylbenzo[d]thiazol-2-yl)piperidine-4-carboxamide.

Another embodiment described herein is a pharmaceutical composition comprising a compound of formula (I):

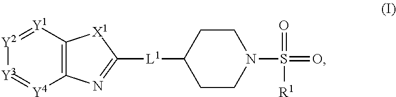

or a pharmaceutically acceptable salt thereof,
wherein:
X¹ is S or O;
Y¹, Y², Y³, and Y⁴ are each C—R² or C—H;
L¹ is

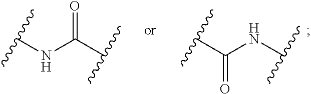 [SMILES: *NC(*)=O ; *NC(*)=O]

R¹ is G¹, —CH₂-G¹, or C₁₋₄alkyl;
R², at each occurrence, is independently C₁₋₄alkyl, C₁₋₄haloalkyl, halogen, C₃₋₄cycloalkyl, —OC₁₋₄alkyl, —OC₁₋₂haloalkyl, or —OC₃₋₄cycloalkyl;
G¹ is a phenyl, a 5- to 9-membered heteroaryl containing 1-3 heteroatoms, a 3- to 7-membered carbocycle, or a 4- to 6-membered heterocycle containing 1-2 heteroatoms, wherein G¹ is optionally substituted with 1 to 3 substituents, each independently R¹¹ or —C₁₋₂alkylene-R¹¹;
R¹¹ is C₁₋₄alkyl, C₁₋₄haloalkyl, C₃₋₄cycloalkyl, halogen, cyano, —OR^{11a}, —N(R^{11a})₂, —NR^{11a}C(O)R^{11b}, —C(O) R^{11b}, —CO₂R^{11b}, or —SO₂R^{11b};
R^{11a}, at each occurrence, are each independently hydrogen, C₁₋₄alkyl, C₁₋₄haloalkyl, or C₃₋₄cycloalkyl; and
R^{11b}, at each occurrence, is independently C₁₋₄alkyl, C₁₋₄haloalkyl, or C₃₋₄cycloalkyl,
and a pharmaceutically acceptable carrier.

Another embodiment described herein is a method for treating a disease or disorder associated with metabolic dysfunction in a mammal, the method comprising administering to the mammal a therapeutically effective amount of the compound of a compound of formula (I), or pharmaceutically acceptable salt thereof, or a pharmaceutical composition comprising a compound of formula (I) and a pharmaceutically acceptable carrier. In one aspect, the disease or disorder is associated with N-acyl phosphatidylethanolamine hydrolyzing phospholipase D (NAPE-PLD) dysfunction. In another aspect, the disease or disorder is obesity, type 2 diabetes, hyperlipidemia, non-alcoholic fatty liver disease, atherosclerosis, hypertriglyceridemia, or hypertension. In another aspect, the disease or disorder is a non-healing wound, a chronic ulcer of the leg or foot, cellulitis or abscess of the leg, or gangrene. In another aspect, the pharmaceutical composition is formulated for topical administration.

Another embodiment described herein is a compound of formula (I), or a pharmaceutically acceptable salt thereof, or a pharmaceutical composition comprising a compound of formula (I) and a pharmaceutically acceptable carrier, for use in the treatment of a disease or disorder associated with metabolic dysfunction in a mammal.

Another embodiment described herein is the use of a compound of formula (I), or pharmaceutically acceptable salt thereof, or a pharmaceutical composition comprising a compound of formula (I) and a pharmaceutically acceptable carrier, for the preparation of a medicament for the treatment of a disease or disorder associated with metabolic dysfunction in a mammal.

### DESCRIPTION OF THE DRAWINGS

FIG. 1 shows a bar graph illustrating that bone marrow derived macrophages (BMDMϕs) from Nape-pld^{-/-} mice have increased tumor necrosis factor alpha (TNFα) expression in response to Isolevuglandin-modified phosphatidylethanolamine (IsoLG-PE).

FIG. 2 shows a bar graph depicting that BMDMϕs from Nape-pld^{-/-} mice have reduced efferocytosis capacity.

FIG. 3 shows the chemical structures of the fluorescent probes PED-A1 and fluorogenic amide/ether-N-acylphosphatidylethanolamine (flame-NAPE).

FIG. 4A-B show scatterplots illustrating that phospholipase activity in HepG2 cells measured using flame-NAPE is sensitive to NAPE-PLD inhibition but not PED-A1 inhibition, whereas phospholipase activity measured by PED-A1 is sensitive to both. HepG2 cells in 96-well plates were treated with 10 μM tetrahydrolipstatin (THL, a pan-lipase inhibitor) and/or 15 μM bithionol (Bith, a NAPE-PLD inhibitor) prior to the addition of either PED-A1 (FIG. 4A) or flame-NAPE (FIG. 4B) (4 μM). Representative 50 min fluorescence time course (1 read per minute) for each treatment. Similar time course obtained on two separate days. Symbols represent average of 6-8 replicate wells for each treatment.

FIG. 5 shows a bar graph illustrating that 10 μM VU534 increases NAPE-PLD activity in primary BMDMϕ, and that Bith inhibits NAPE-PLD activity in primary BMDMϕ.

FIG. 6 shows a bar graph depicting that 10 μM VU534 increases efferocytosis capacity, and that Bith reduced efferocytosis.

FIG. 7 shows a scatterplot illustrating that VU534 enhances NAPE-PLD activity in HEPG2 cells.

FIG. 8 shows a scatterplot illustrating that VU517 enhances NAPE-PLD activity in HEPG2 cells.

FIG. 9 shows a scatterplot illustrating that VU575 enhances NAPE-PLD activity in HEPG2 cells.

FIG. 10 shows a scatterplot depicting that VU534 increases the catalytic rate of NAPE-PLD. RFU: relative fluorescence units.

FIG. 11 shows the Molecular Dynamics simulation of the NAPE-PLD active site before (pink) and 50 ns after (green) VU534 binding. Spheres: catalytic zinc. Ball and stick: phosphatidylethanolamine (PE, used as surrogate for NAPE).

FIG. 12 shows a scatterplot depicting that VU534 inhibits soluble epoxide hydrolase (sEH) activity.

FIG. 13 shows a scatterplot depicting that VU534 does not inhibit Fatty Amide Acid Hydrolase (FAAH) activity.

FIG. 14 shows a bar graph of the effects of various BT-PSP analogs (10 μM) on NAPE-PLD activity of RAW264.7 macrophages.

FIG. 15 shows the biosynthesis of N-acyl-ethanolamides via NAPE-PLD. N-acyl-ethanolamides (NAEs) including palmitoylethanolamide (PEA) and oleoylethanolamide (OEA) are formed by NAPE-PLD pathway in a two-step process. First, PE N-acyltransferases transfer an acyl chain from phosphatidylethanolamine (PC) to the nitrogen of phosphatidylethanolamine (PE) to generate N-acyl-phosphatidylethanolamines (NAPEs), and lysophosphatidylcholine (lysoPC). Then NAPE-PLD cleaves NAPE at the distal phosphodiester bond to generate the NAE and phosphatidic acid (PA). NAEs then act on receptors including PPARa, GPR119, and GPR55 to exert biological effects. NAEs are rapidly inactivated by fatty acid amide hydrolase (FAAH) and N-acylethanolamide acid amidase (NAAA) by their degradation to ethanolamine and free fatty acid.

FIG. 16A-C show high throughput screening identifies potential NAPE-PLD activators. FIG. 16A shows a schematic representation of the HTS assay which detects Nape-Pld activity by the fluorescence resulting from the hydrolysis of the fluorogenic NAPE analog PED-A1. Intact PED-A1 only weakly fluoresces due to internal quenching by its dinitrophenyl moiety. Nape-pld hydrolysis of PED-A1 generates dinitrophenyl-hexanoyl-ethanolamide (DNP-EA and highly fluorescent BODIPY-labeled phosphatidic acid (BODIPY-PA). FIG. 16B shows sample activity curves from HTS for controls, a representative activator hit, and a representative inhibitor hit. The shaded region represents the time period used in scoring fluorescence changes for all compounds. FIG. 16C shows B-scores of various test library compounds in the HTS assay. Compounds with B-scores ≥3 (activators) or ≤−3 (inhibitors) were deemed to be potential hits.

FIG. 17A-C show VU534 and VU533 increase the NAPE-PLD activity of RAW264.7 macrophages. FIG. 17A shows the effect of graded concentrations of VU534 (left panel), VU533 (middle panel), and VU233 (right panel) on Nape-pld activity in RAW264.7 cells, measured using PED-A1. Each compound was tested on at least two separate days and the individual replicates from each day normalized to vehicle control and then combined (mean±SEM, n=4-11). 1-way ANOVA p<0.0001 for VU534, p<0.0001 for VU533, and p=not significant for VU233. *p<0.05 vs. 0 μM, Dunnet's multiple comparison test. Non-linear regression with variable slope (four parameter) was used to calculate EC₅₀ and Eₘₐₓ for VU534 EC₅₀ 6.6 μM (95% CI 2.6 to 11.2 μM), Eₘₐₓ 1.6-fold (95% CI n.d.); and VU533 EC₅₀ 2.5 μM (95% CI 1.4 to 6.1 μM), Eₘₐₓ 2.2-fold (95% CI 2.0 to 2.7-fold). FIG. 17B shows the correlation between maximal efficacy of 19 BT-PSPs and analogs in the recombinant Nape-pld assay and their efficacy at 30 μM in cultured RAW264.7 cells. Simple linear regression. Slope=0.5455, R² 0.3394 p=0.0089 for slope significantly non-zero. FIG. 17C shows bithionol, a Nape-pld inhibitor, blocks increased Nape-pld activity in RAW264.7 cells induced by Compound 8. 1-way ANOVA p<0.0001 Sidak's multiple comparison test, a p<0.05 vs 0 μM Bith with 0 μM VU534 group, ^{b}p<0.05 vs. 0 μM Bith with 20 μM VU534 group.

FIG. 18A-B show VU534 and VU533 activate human NAPE-PLD. FIG. 18A shows the effect of graded concentrations of VU534, VU533, and VU233 on activity of recombinant human NAPE-PLD with PED-A1 as substrate. Mean±SEM, n=3. Non-linear regression with variable slope (four parameter) was used to calculate EC₅₀ and Eₘₐₓ. VU534 EC₅₀ 0.93 μM (95% CI 0.63 to 1.39 μM), Eₘₐₓ 1.8-fold (95% CI 1.8 to 1.9-fold); VU533 EC₅₀ 0.20 μM (95% CI 0.12 to 0.32 μM), Eₘₐₓ 1.9-fold (95% CI 1.8 to 2.0-fold); VU233 not calculable. FIG. 18B shows the effect of graded concentrations of VU534, VU533, and VU233 on NAPE-PLD activity of HepG2 cells measured using flame-NAPE as substrate. Each compound was tested on two separate days and the individual replicates from each day normalized to vehicle control and then combined (mean±SEM, n=4-6); VU534 EC₅₀ 1.5 μM (95% CI 0.6 to 2.8 μM), Eₘₐₓ 1.6-fold activity (95% CI 1.5 to 1.8-fold); VU533 EC₅₀ 3.0 μM (1.4 to 5.7 μM), Eₘₐₓ 1.6-fold (95% CI 1.5 to 1.8) fold; VU233 not calculable.

FIG. 19A-D show additional characterization of NAPE-PLD modulation by VU534. FIG. 19A shows the activity of recombinant mouse NAPE-PLD using N-oleoyl-phosphatidylethanolamine (NOPE) as substrate and measuring OEA and NOPE by LC/MS/MS. Ratio of OEA to NAPE was normalized to 0 μM compound control. The assays of VU534 and VU233 were performed using the same 0 μM compound replicates. 1-way ANOVA VU534 p<0.0001, VU533 p=0.007, VU233 p=0.0547; Dunnett's multiple comparison test for individual compounds **p=0.0074, ***p=0.0005, ****p<0.0001. FIG. 19B shows Michaelis-Menten analysis using flame-NAPE as substrate for recombinant mouse NAPE-PLD with or without VU534. Non-linear regression curves (allosteric sigmoidal) were used to calculate K_{1/2} and Vₘₐₓ. FIG. 19C shows in vitro competition assay for effects on flame-NAPE hydrolysis by recombinant mouse Nape-pld. 1-way ANOVA, p<0.0001. Groups sharing letters do not significantly differ from each other in Tukey multiple comparisons test. FIG. 19D shows data from in vitro competition assay data normalized to the value for 0 μM LEI-401 with 5 μM VU534 for all samples with 5 μM VU534 (VU534) or 0 μM LEI-401 with vehicle for all samples with no VU534 (Vehicle). Samples treated with the same concentration of LEI-401, with or without 5 μM VU534, did not significantly differ, Sidak's multiple comparisons test.

FIG. 20A-B show evaluation of off-target effects on FAAH and sEH. FIG. 20A shows the effects of graded concentrations of VU534, VU533, or VU233 on activity of Fatty Acid Amide Hydrolase (FAAH). 1-way ANOVA VU534 p=0.0007; VU533 p<0.0001; and VU233 p<0.0001; *p<0.05 vs. 0 μM, Dunnett's multiple comparison test for individual compounds. FIG. 20B shows the effects of graded concentrations of VU534, VU533, or VU233 on activity of soluble Epoxide Hydrolase (sEH). 1-way ANOVA VU534 p<0.0001; VU533 p<0.0001; and VU233 p<0.0001; *p<0.05 vs. 0 μM, Dunnett's multiple comparison test for individual compounds.

FIG. 21A-C show modulation of NAPE-PLD modulates efferocytosis by macrophages. FIG. 21A shows BMDM from wild-type mice were treated with 10 μM VU534, VU533, VU233 or bithionol (Bith) for 6 h prior to initiation of efferocytosis assay. 1-way ANOVA p=0.0004. Dunnett's multiple comparison's test p value shown for each comparison. Data shown from one representative experimental day. All compounds were tested in two to six separate experimental days. FIG. 21B shows BMDM from wild-type mice were treated with 10 μM VU534 or with sEH inhibitor AUDA (10 μM) or TPPU (10 μM) for 6 h prior to initiation of efferocytosis assay. 1-way ANOVA p=0.0004. Dunnett's multiple comparisons test p value shown for each comparison. Data shown from one representative experimental day of two total experimental days. FIG. 21C shows BMDM from wild-type (WT) or Napepld^{-/-} (KO) mice were treated with vehicle (veh) or 10 UM VU534 for 6 h prior to initiation of the efferocytosis assay. 1-way ANOVA p<0.0001, Dunnett's multiple comparison p value shown for each comparison. Data shown from one representative experimental day of three total experimental days.

### DETAILED DESCRIPTION

Disclosed herein are allosteric activators or positive allosteric modulators of N-acyl phosphatidylethanolamine hydrolyzing phospholipase D (NAPE-PLD). The activators or modulators may be compounds of formula (I). Compounds of formula (I) may exhibit selectivity for NAPE-PLD over other enzymes that regulate metabolism and inflammation. Compounds of formula (I) can be used to treat or prevent diseases and disorders associated with NAPE-PLD by stimulating or increasing NAPE-PLD activity. Reduced NAPE-PLD expression has been implicated in several different diseases and disorders associated with metabolic dysfunction including, but not limited to, obesity, type 2 diabetes, hyperlipidemia, non-alcoholic fatty liver disease, atherosclerosis, hypertriglyceridemia, and hypertension. One strategy to selectively bind and activate or modulate NAPE-PLD includes identifying allosteric sites which may be amenable to activation or modulation by a small molecule. In particular, activation or positive allosteric modulation of NAPE-PLD can result in the activation of processes governed by NAPE-PLD and provide therapeutic benefits for disorders associated with NAPE-PLD dysfunction.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to +10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “pharmaceutically acceptable carrier” or “pharmaceutically acceptable excipient” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see e.g., Remington's Pharmaceutical Sciences, 18^{th} ed., Mack Printing Company, 1990, pp. 1289-1329). Except insofar as any conventional carrier is incompatible with the active ingredient, its use in the therapeutic or pharmaceutical compositions is contemplated.

As used herein, the terms “salt” or “salts” refers to an acid addition or base addition salt of a compound of the invention. “Salts” include in particular “pharmaceutical acceptable salts.” The term “pharmaceutically acceptable salts” refers to salts that retain the biological effectiveness and properties of the compounds of this invention and, which typically are not biologically or otherwise undesirable. In many cases, the compounds described herein are capable of forming acid and/or base salts by virtue of the presence of amino and/or carboxyl groups or groups similar thereto.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

As used herein, a “metabolic disease” or a “metabolic disorder” refers to diseases and disorders that occur due to an in vivo metabolic dysfunction. The metabolic disease or disorder is generally caused by the imbalance of carbohydrates, lipids, proteins, vitamins, electrolytes, water, and the like. Representative examples thereof include obesity, diabetes, hyperlipidemia, arteriosclerosis, and the like, all of which are caused by a high fat diet.

As used herein, “treating or preventing a metabolic disease or disorder” includes alleviating and mitigating a metabolic disease or disorder, and improving symptoms, and, also includes lowering the probability of getting a metabolic disease or disorder.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy,” as used herein, refers to a group-O-alkyl. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond.

The term “alkoxyalkyl,” as used herein, refers to an alkoxy group, as defined herein, appended to the parent molecular moiety through an alkyl group, as defined herein.

The term “alkoxyfluoroalkyl,” as used herein, refers to an alkoxy group, as defined herein, appended to the parent molecular moiety through a fluoroalkyl group, as defined herein.

The term “alkylene,” as used herein, refers to a divalent group derived from a straight or branched chain hydrocarbon of 1 to 10 carbon atoms, for example, of 2 to 5 carbon atoms. Representative examples of alkylene include, but are not limited to, —CH₂—, —CD₂-, —CH₂CH₂—, —CH₂CH₂CH₂—, —CH₂CH₂CH₂CH₂—, and —CH₂CH₂CH₂CH₂CH₂—.

The term “alkylamino,” as used herein, means at least one alkyl group, as defined herein, is appended to the parent molecular moiety through an amino group, as defined herein.

The term “amide,” as used herein, means —C(O)NR— or —NRC(O)—, wherein R may be hydrogen, alkyl, cycloalkyl, aryl, heteroaryl, heterocycle, alkenyl, or heteroalkyl.

The term “aminoalkyl,” as used herein, means at least one amino group, as defined herein, is appended to the parent molecular moiety through an alkylene group, as defined herein.

The term “amino,” as used herein, means —NRₓR_{y}, wherein Rₓ and R_{y} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NRₓ—, wherein Rₓ may be hydrogen, alkyl, cycloalkyl, aryl, heteroaryl, heterocycle, alkenyl, or heteroalkyl.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “cyanoalkyl,” as used herein, means at least one-CN group, is appended to the parent molecular moiety through an alkylene group, as defined herein.

The term “cyanofluoroalkyl,” as used herein, means at least one-CN group, is appended to the parent molecular moiety through a fluoroalkyl group, as defined herein.

The term “cycloalkoxy,” as used herein, refers to a cycloalkyl group, as defined herein, appended to the parent molecular moiety through an oxygen atom.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl).

Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “cycloalkenyl” or “cycloalkene,” as used herein, means a non-aromatic monocyclic or multicyclic ring system containing all carbon atoms as ring members and at least one carbon-carbon double bond and preferably having from 5-10 carbon atoms per ring. The term “cycloalkenyl” is used herein to refer to a cycloalkene when present as a substituent. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl).

Exemplary monocyclic cycloalkenyl rings include cyclopentenyl, cyclohexenyl or cycloheptenyl. Exemplary monocyclic cycloalkenyl rings include cyclopentenyl, cyclohexenyl or cycloheptenyl.

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The terms cycloalkylene and heterocyclylene refer to divalent groups derived from the base ring, i.e., cycloalkane, heterocycle. For purposes of illustration, examples of cycloalkylene and heterocyclylene include, respectively,

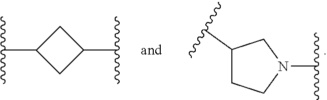 [SMILES: *C1CC(*)C1 ; *C1CCN(*)C1]

Cycloalkylene and heterocyclylene include a geminal divalent groups such as 1,1-C₃₋₆cycloalkylene

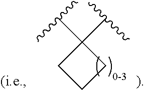

A further example is 1,1-cyclopropylene

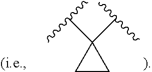 [SMILES: *C1(*)CC1]

The term “fluoroalkyl,” as used herein, means an alkyl group, as defined herein, in which one, two, three, four, five, six, seven or eight hydrogen atoms are replaced by fluorine. Representative examples of fluoroalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

The term “fluoroalkylene,” as used herein, means an alkylene group, as defined herein, in which one, two, three, four, five, six, seven or eight hydrogen atoms are replaced by fluorine. Representative examples of fluoroalkyl include, but are not limited to —CF₂—, —CH₂CF₂—, 1,2-difluoroethylene, 1,1,2,2-tetrafluoroethylene, 1,3,3,3-tetrafluoropropylene, 1,1,2,3,3-pentafluoropropylene, and perfluoropropylene such as 1,1,2,2,3,3-hexafluoropropylene.

The term “halogen” or “halo,” as used herein, means CI, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

The term “heteroaryl,” as used herein, refers to an aromatic monocyclic heteroatom-containing ring (monocyclic heteroaryl) or a bicyclic ring system containing at least one monocyclic heteroaromatic ring (bicyclic heteroaryl). The term “heteroaryl” is used herein to refer to a heteroarene when present as a substituent. The monocyclic heteroaryl are five or six membered rings containing at least one heteroatom independently selected from the group consisting of N, O and S (e.g., 1, 2, 3, or 4 heteroatoms independently selected from O, S, and N). The five membered aromatic monocyclic rings have two double bonds, and the six membered aromatic monocyclic rings have three double bonds. The bicyclic heteroaryl is an 8- to 12-membered ring system and includes a fused bicyclic heteroaromatic ring system (i.e., 10π electron system) such as a monocyclic heteroaryl ring fused to a 6-membered arene (e.g., quinolin-4-yl, indol-1-yl), a monocyclic heteroaryl ring fused to a monocyclic heteroarene (e.g., naphthyridinyl), and a phenyl fused to a monocyclic heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 10π electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3,2-b]pyridinyl). The bicyclic heteroaryl is attached to the parent molecular moiety at an aromatic ring atom. Other representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, tetrahydroisoquinolinyl, 7-oxabicyclo[2.2.1]heptanyl, hexahydro-2H-cyclopenta[b]furanyl, 2-oxaspiro[3.3]heptanyl, 3-oxaspiro[5.5]undecanyl, 6-oxaspiro[2.5]octan-1-yl, and 3-oxabicyclo[3.1.0]hexan-6-yl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocyclyls are connected to the parent molecular moiety at a non-aromatic ring atom.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

The term “hydroxyfluoroalkyl,” as used herein, means at least one —OH group, is appended to the parent molecular moiety through a fluoroalkyl group, as defined herein.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

### Compounds

### Compounds of Formula (I)

In one aspect, the invention provides compounds of formula (I), wherein X¹, X³, Y¹, Y², Y³, Y⁴, L¹, G¹, R¹, R², R¹¹, R^{11a}, and R^{11b} are as defined herein.

Unsubstituted or substituted rings (i.e., optionally substituted) such as aryl, heteroaryl, etc. are composed of both a ring system and the ring system's optional substituents. Accordingly, the ring system may be defined independently of its substituents, such that redefining only the ring system leaves any previous optional substituents present. For example, a 5- to 12-membered heteroaryl with optional substituents may be further defined by specifying the ring system of the 5- to 12-membered heteroaryl is a 5- to 6-membered heteroaryl (i.e., 5- to 6-membered heteroaryl ring system), in which case the optional substituents of the 5- to 12-membered heteroaryl are still present on the 5- to 6-membered heteroaryl, unless otherwise expressly indicated.

In the following, embodiments of the invention are disclosed. The first embodiment is denoted E1, another embodiment is denoted E2 and so forth.

E1. A compound of formula (I):

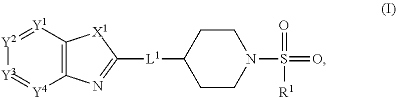

or a pharmaceutically acceptable salt thereof,
wherein:
X¹ is S or O;
Y¹ and Y³ are each C—R² and Y² and Y⁴ are each C—H, or
Y¹ and Y³ are each C—H and Y² and Y⁴ are each C—R²;
L¹ is

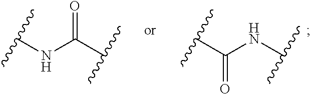 [SMILES: *NC(*)=O ; *NC(*)=O]

R¹ is G¹, —CH₂-G¹, or C₁₋₄alkyl;
R², at each occurrence, is independently C₁₋₄alkyl, C₁₋₄haloalkyl, halogen, C₃₋₄cycloalkyl, —OC₁₋₄alkyl, —OC₁₋₂haloalkyl, or —OC₃₋₄cycloalkyl;
G¹ is a phenyl, a 5- to 9-membered heteroaryl containing 1-3 heteroatoms, a 3- to 7-membered carbocycle, or a 4- to 6-membered heterocycle containing 1-2 heteroatoms, wherein G¹ is optionally substituted with 1 to 3 substituents, each independently R¹¹ or —C₁₋₂alkylene-R¹¹;
R¹¹ is C₁₋₄alkyl, C₁₋₄haloalkyl, C₃₋₄cycloalkyl, halogen, cyano, —OR^{11a}, —N(R^{11a})₂, —NR^{11a}C(O)R^{11b}, —C(O) R^{11b}, —CO₂R^{11b}, or —SO₂R^{11b};
R^{11a}, at each occurrence, are each independently hydrogen, C₁₋₄alkyl, C₁₋₄haloalkyl, or C₃₋₄cycloalkyl; and
R^{11b}, at each occurrence, is independently C₁₋₄alkyl, C₁₋₄haloalkyl, or C₃₋₄cycloalkyl.

E2. The compound of E1, or a pharmaceutically acceptable salt thereof, wherein X¹ is S.

E3. The compound of E1 or E2, or a pharmaceutically acceptable salt thereof, wherein Y¹ and Y³ are each C—C₁₋₄alkyl and Y² and Y⁴ are each C—H, or, where Y¹ and Y³ are each C—H and Y² and Y⁴ are each C—C₁₋₄alkyl.

E4. The compound of any one of E1-E3, or a pharmaceutically acceptable salt thereof, wherein Y¹ and Y³ are each C—CH₃, and Y² and Y⁴ are each C—H.

E5. The compound of any one of E1-E3, or a pharmaceutically acceptable salt thereof, wherein Y¹ and Y³ are each C—H, and Y² and Y⁴ are each C—CH₃.

E6. The compound of any one of E1-E5, or a pharmaceutically acceptable salt thereof, wherein L¹ is

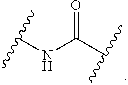 [SMILES: *NC(*)=O]

E7. The compound of any one of E1-E6, or a pharmaceutically acceptable salt thereof, wherein R¹ is G¹.

E8. The compound of E7, or a pharmaceutically acceptable salt thereof, wherein G¹ is the optionally substituted phenyl.

E9. The compound of E8, or a pharmaceutically acceptable salt thereof, wherein G¹ is

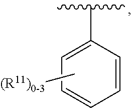

wherein R¹¹, at each occurrence, is independently halogen, cyano, C₁₋₄alkyl, C₁₋₂haloalkyl, —OC₁₋₄alkyl, or —OC₁₋₂haloalkyl.

E10. The compound of E9, or a pharmaceutically acceptable salt thereof, wherein G¹ is

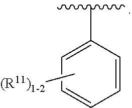

E11. The compound of E10, or a pharmaceutically acceptable salt thereof, wherein G¹ is

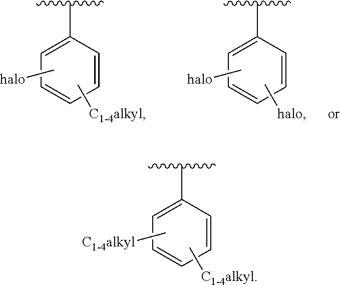

E12. The compound of E11, or a pharmaceutically acceptable salt thereof, wherein G¹ is

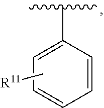

where R¹¹ is halogen, cyano, C₁₋₄alkyl, C₁₋₂haloalkyl, —OC₁₋₄alkyl, or —OC₁₋₂haloalkyl.

E13. The compound of E7, or a pharmaceutically acceptable salt thereof, wherein G¹ is the optionally substituted 5- to 9-membered heteroaryl.

E14. The compound of E13, or a pharmaceutically acceptable salt thereof, wherein the ring system of the optionally substituted 5- to 9-membered heteroaryl is a pyridinyl, a pyrimidinyl, a pyrazolyl, or a benzo[c][1,2,5]oxadiazolyl.

E15. The compound of E14, or a pharmaceutically acceptable salt thereof, wherein R¹ is

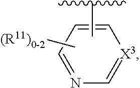

wherein X³ is C—H, or N, and R¹¹, at each occurrence, is independently halogen, cyano, C₁₋₄alkyl, C₁₋₂haloalkyl, —OC₁₋₄alkyl, or —OC₁₋₂haloalkyl.

E16. The compound of E15, or a pharmaceutically acceptable salt thereof, wherein R¹ is

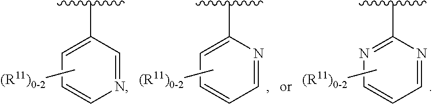

E17. The compound of claim E16, or a pharmaceutically acceptable salt thereof, wherein R¹ is

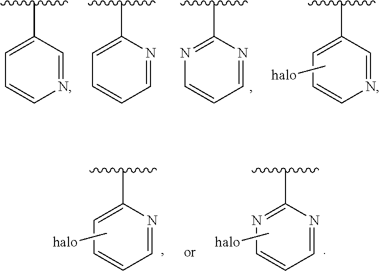

E18. The compound of any one of any one of E1-E6, or a pharmaceutically acceptable salt thereof, wherein R¹ is —CH₂-G¹.

E19. The compound of E7 or E18, or a pharmaceutically acceptable salt thereof, wherein G¹ is the optionally substituted 3- to 7-membered carbocycle.

E20. The compound of E19, or a pharmaceutically acceptable salt thereof, wherein the ring system of the optionally substituted 3- to 7-membered carbocycle is a 3- or 6-membered carbocycle.

E21. The compound of E20, or a pharmaceutically acceptable salt thereof, wherein R¹ is

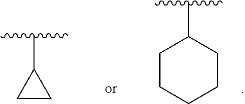

E22. The compound of any one of E1-E6, or a pharmaceutically acceptable salt thereof, wherein R¹ is C₁₋₄alkyl.

E23. The compound of E22, or a pharmaceutically acceptable salt thereof, wherein R¹ is methyl.

E24. The compound of E1, wherein the compound is
1-((3-chloro-4-fluorophenyl)sulfonyl)-N-(5,7-dimethylbenzo[d]thiazol-2-yl)piperidine-4-carboxamide;
N-(4,6-dimethylbenzo[d]thiazol-2-yl)-1-((4-fluoro-2-methylphenyl)sulfonyl)piperidine-4-carboxamide;
N-(4,6-dimethylbenzo[d]thiazol-2-yl)-1-(pyridin-3-ylsulfonyl)piperidine-4-carboxamide;
N-(4,6-dimethylbenzo[d]thiazol-2-yl)-1-((1-methyl-1H-pyrazol-4-yl)sulfonyl)piperidine-4-carboxamide;
1-((2-chlorophenyl)sulfonyl)-N-(4,6-dimethylbenzo[d]thiazol-2-yl)piperidine-4-carboxamide;
N-(4,6-dimethylbenzo[d]thiazol-2-yl)-1-(o-tolylsulfonyl)piperidine-4-carboxamide;
N-(4,6-dimethylbenzo[d]thiazol-2-yl)-1-((3-fluorophenyl)sulfonyl)piperidine-4-carboxamide;
1-((3-chlorophenyl)sulfonyl)-N-(4,6-dimethylbenzo[d]thiazol-2-yl)piperidine-4-carboxamide;
N-(4,6-dimethylbenzo[d]thiazol-2-yl)-1-(m-tolylsulfonyl)piperidine-4-carboxamide;
1-((4-cyanophenyl)sulfonyl)-N-(4,6-dimethylbenzo[d]thiazol-2-yl)piperidine-4-carboxamide;
N-(4,6-dimethylbenzo[d]thiazol-2-yl)-1-((4-(trifluoromethoxy)phenyl)sulfonyl)piperidine-4-carboxamide;
1-((2-chloro-4-fluorophenyl)sulfonyl)-N-(4,6-dimethylbenzo[d]thiazol-2-yl)piperidine-4-carboxamide;
N-(4,6-dimethylbenzo[d]thiazol-2-yl)-1-((4-fluoro-2-methylphenyl)sulfonyl)piperidine-4-carboxamide;
N-(4,6-dimethylbenzo[d]thiazol-2-yl)-1-((6-fluoropyridin-3-yl)sulfonyl)piperidine-4-carboxamide;
N-(4,6-dimethylbenzo[d]thiazol-2-yl)-1-((5-fluoropyridin-2-yl)sulfonyl)piperidine-4-carboxamide;
N-(4,6-dimethylbenzo[d]thiazol-2-yl)-1-((5-fluoropyrimidin-2-yl)sulfonyl)piperidine-4-carboxamide;
N-(4,6-dimethylbenzo[d]thiazol-2-yl)-1-((2-fluoropyridin-3-yl)sulfonyl)piperidine-4-carboxamide;
N-(4,6-dimethylbenzo[d]thiazol-2-yl)-1-((3-fluoropyridin-2-yl)sulfonyl)piperidine-4-carboxamide;
N-(4,6-dimethylbenzo[d]thiazol-2-yl)-1-((3-fluoro-4-methylphenyl)sulfonyl)piperidine-4-carboxamide;
1-(cyclohexylsulfonyl)-N-(4,6-dimethylbenzo[d]thiazol-2-yl)piperidine-4-carboxamide;
1-((3,4-dichlorophenyl)sulfonyl)-N-(4,6-dimethylbenzo[d]thiazol-2-yl)piperidine-4-carboxamide;
1-((2,4-difluorobenzyl)sulfonyl)-N-(4,6-dimethylbenzo[d]thiazol-2-yl)piperidine-4-carboxamide;
1-(cyclopropylsulfonyl)-N-(4,6-dimethylbenzo[d]thiazol-2-yl)piperidine-4-carboxamide;
1-((cyclopropylmethyl)sulfonyl)-N-(4,6-dimethylbenzo[d]thiazol-2-yl)piperidine-4-carboxamide;
1-(benzo[c][1,2,5]oxadiazol-4-ylsulfonyl)-N-(4,6-dimethylbenzo[d]thiazol-2-yl)piperidine-4-carboxamide;
1-((3-chloro-4-fluorophenyl)sulfonyl)-N-(5,7-dimethylbenzo[d]thiazol-2-yl)piperidine-4-carboxamide;
N-(5,7-dimethylbenzo[d]thiazol-2-yl)-1-((4-fluoro-2-methylphenyl)sulfonyl)piperidine-4-carboxamide;
N-(5,7-dimethylbenzo[d]thiazol-2-yl)-1-(pyridin-3-ylsulfonyl)piperidine-4-carboxamide;
N-(5,7-dimethylbenzo[d]thiazol-2-yl)-1-((1-methyl-1H-pyrazol-4-yl)sulfonyl)piperidine-4-carboxamide;
1-((2-chlorophenyl)sulfonyl)-N-(5,7-dimethylbenzo[d]thiazol-2-yl)piperidine-4-carboxamide;
N-(5,7-dimethylbenzo[d]thiazol-2-yl)-1-(o-tolylsulfonyl)piperidine-4-carboxamide;
N-(5,7-dimethylbenzo[d]thiazol-2-yl)-1-((3-fluorophenyl)sulfonyl)piperidine-4-carboxamide;
1-((3-chlorophenyl)sulfonyl)-N-(5,7-dimethylbenzo[d]thiazol-2-yl)piperidine-4-carboxamide;
N-(5,7-dimethylbenzo[d]thiazol-2-yl)-1-(m-tolylsulfonyl)piperidine-4-carboxamide;
1-((4-cyanophenyl)sulfonyl)-N-(5,7-dimethylbenzo[d]thiazol-2-yl)piperidine-4-carboxamide;
N-(5,7-dimethylbenzo[d]thiazol-2-yl)-1-((4-(trifluoromethoxy)phenyl)sulfonyl)piperidine-4-carboxamide;
1-((2-chloro-4-fluorophenyl)sulfonyl)-N-(5,7-dimethylbenzo[d]thiazol-2-yl)piperidine-4-carboxamide;
N-(5,7-dimethylbenzo[d]thiazol-2-yl)-1-((4-fluoro-2-methylphenyl)sulfonyl)piperidine-4-carboxamide;
N-(5,7-dimethylbenzo[d]thiazol-2-yl)-1-((6-fluoropyridin-3-yl)sulfonyl)piperidine-4-carboxamide;
N-(5,7-dimethylbenzo[d]thiazol-2-yl)-1-((5-fluoropyridin-2-yl)sulfonyl)piperidine-4-carboxamide;
N-(5,7-dimethylbenzo[d]thiazol-2-yl)-1-((5-fluoropyrimidin-2-yl)sulfonyl)piperidine-4-carboxamide;
N-(5,7-dimethylbenzo[d]thiazol-2-yl)-1-((2-fluoropyridin-3-yl)sulfonyl)piperidine-4-carboxamide;
N-(5,7-dimethylbenzo[d]thiazol-2-yl)-1-((3-fluoropyridin-2-yl)sulfonyl)piperidine-4-carboxamide;
N-(5,7-dimethylbenzo[d]thiazol-2-yl)-1-((3-fluoro-4-methylphenyl)sulfonyl)piperidine-4-carboxamide;
1-(cyclohexylsulfonyl)-N-(5,7-dimethylbenzo[d]thiazol-2-yl)piperidine-4-carboxamide;
1-((3,4-dichlorophenyl)sulfonyl)-N-(5,7-dimethylbenzo[d]thiazol-2-yl)piperidine-4-carboxamide;
1-((2,4-difluorobenzyl)sulfonyl)-N-(5,7-dimethylbenzo[d]thiazol-2-yl)piperidine-4-carboxamide;
1-(cyclopropylsulfonyl)-N-(5,7-dimethylbenzo[d]thiazol-2-yl)piperidine-4-carboxamide;
1-((cyclopropylmethyl)sulfonyl)-N-(5,7-dimethylbenzo[d]thiazol-2-yl)piperidine-4-carboxamide; or
1-(benzo[c][1,2,5]oxadiazol-4-ylsulfonyl)-N-(5,7-dimethylbenzo[d]thiazol-2-yl)piperidine-4-carboxamide.

E25. A pharmaceutical composition comprising the compound of any one of E1-E24 and a pharmaceutically acceptable carrier.

E26. A pharmaceutical composition comprising a compound of formula (I)

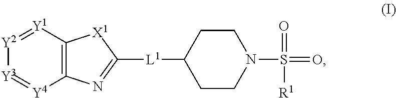

or a pharmaceutically acceptable salt thereof,
wherein:
X¹ is S or O;
Y¹, Y², Y³, and Y⁴ are each C—R² or C—H;
L¹ is

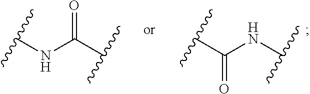 [SMILES: *NC(*)=O ; *NC(*)=O]

R¹ is G¹, —CH₂-G¹, or C₁₋₄alkyl;
R², at each occurrence, is independently C₁₋₄alkyl, C₁₋₄haloalkyl, halogen, C₃₋₄cycloalkyl, —OC₁₋₄alkyl, —OC₁₋₂haloalkyl, or —OC₃₋₄cycloalkyl;
G¹ is a phenyl, a 5- to 9-membered heteroaryl containing 1-3 heteroatoms, a 3- to 7-membered carbocycle, or a 4- to 6-membered heterocycle containing 1-2 heteroatoms, wherein G¹ is optionally substituted with 1 to 3 substituents, each independently R¹¹ or —C₁₋₂alkylene-R¹¹;
R¹¹ is C₁₋₄alkyl, C₁₋₄haloalkyl, C₃₋₄cycloalkyl, halogen, cyano, —OR^{11a}, —N(R^{11a})₂, —NR^{11a}C(O)R^{11b}, —C(O) R^{11b}, —CO₂R^{11b}, or —SO₂R^{11b};
R^{11a}, at each occurrence, are each independently hydrogen, C₁₋₄alkyl, C₁₋₄haloalkyl, or C₃₋₄cycloalkyl; and
R^{11b}, at each occurrence, is independently C₁₋₄alkyl, C₁₋₄haloalkyl, or C₃₋₄cycloalkyl;
and a pharmaceutically acceptable carrier.

E27. A method for treating a disease or disorder associated with metabolic dysfunction in a mammal, the method comprising administering to the mammal a therapeutically effective amount of the compound of any one of E1-E24, or pharmaceutically acceptable salt thereof, or the pharmaceutical composition of E25 or E26.

E28. The method of E27, wherein the disease or disorder is associated with N-acyl phosphatidylethanolamine hydrolyzing phospholipase D (NAPE-PLD) dysfunction.

E29. The method of E27 or E28, wherein the disease or disorder is obesity, type 2 diabetes, hyperlipidemia, non-alcoholic fatty liver disease, atherosclerosis, hypertriglyceridemia, or hypertension.

E30. The method of E28 or E29, wherein the disease or disorder is a non-healing wound, a chronic ulcer of the leg or foot, cellulitis or abscess of the leg, or gangrene.

E31. The pharmaceutical composition of E25 or E26, wherein the pharmaceutical composition is formulated for topical administration.

E32. The compound of any one of E1-E24, or pharmaceutically acceptable salt thereof, or the pharmaceutical composition of E25 or E26, for use in the treatment of a disease or disorder associated with metabolic dysfunction in a mammal.

E33. The use of the compound of any one of E1-E24, or pharmaceutically acceptable salt thereof, or the pharmaceutical composition of E25 or E26, for the preparation of a medicament for the treatment of a disease or disorder associated with metabolic dysfunction in a mammal.

Compound names can be assigned by using Struct=Name naming algorithm as part of CHEMDRAW® ULTRA.

The compound may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45:13-30. The disclosure contemplates various stereoisomers and mixtures thereof and these are specifically included within the scope of this invention. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

It should be understood that the compound may possess tautomeric forms, as well as geometric isomers, and that these also constitute an aspect of the invention.

In the compounds of formula (I), and any subformulas, any “hydrogen” or “H,” whether explicitly recited or implicit in the structure, encompasses hydrogen isotopes 1H (protium) and 2H (deuterium).

The present disclosure also includes isotopically-labeled compounds (e.g., deuterium labeled), where an atom in the isotopically-labeled compound is specified as a particular isotope of the atom. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively.

Isotopically-enriched forms of compounds of formula (I), or any subformulas, may generally be prepared by conventional techniques known to those skilled in the art or by processes analogous to those described in the accompanying Examples using an appropriate isotopically-enriched reagent in place of a non-isotopically-enriched reagent. The extent of isotopic enrichment can be characterized as a percent incorporation of a particular isotope at an isotopically-labeled atom (e.g., % deuterium incorporation at a deuterium label).

### NAPE-PLD Activators or Positive Allosteric Modulators

The disclosed compounds may act or function as allosteric activators or positive allosteric modulators of N-acyl phosphatidylethanolamine hydrolyzing phospholipase D (NAPE-PLD).

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein the term “a therapeutically effective amount” of a compound described herein refers to an amount of the compound described herein that will elicit the biological or medical response of a subject, for example, reduction or inhibition, or increase or stimulation of an enzyme or a protein activity, or ameliorate symptoms, alleviate conditions, slow or delay disease progression, or prevent a disease, etc. In one non-limiting embodiment, the term “a therapeutically effective amount” refers to the amount of the compound described herein that, when administered to a subject, is effective to (1) at least partially alleviate, inhibit, prevent and/or ameliorate a condition, or a disorder or a disease (i) mediated by NAPE-PLD, or (ii) associated with NAPE-PLD activity, or (iii) characterized by activity (normal or abnormal) of NAPE-PLD; or (2) activate or modulate the activity of NAPE-PLD; or (3) activate or modulate the expression of NAPE-PLD. In another non-limiting embodiment, the term “a therapeutically effective amount” refers to the amount of the compound described herein that, when administered to a cell, or a tissue, or a non-cellular biological material, or a medium, is effective to at least partially activating or modulating the activity of NAPE-PLD; or at least partially activating or modulating the expression of NAPE-PLD.

Compounds of formula (I) may activate NAPE-PLD with an AC₅₀ ranging from about 1 nM to about 30 μM. The compounds may have an AC₅₀ of about 30 μM, about 29 μM, about 28 μM, about 27 μM, about 26 μM, about 25 μM, about 24 μM, about 23 μM, about 22 μM, about 21 μM, about 20 μM, about 19 μM, about 18 μM, about 17 μM, about 16 μM, about 15 μM, about 14 μM, about 13 μM, about 12 μM, about 11 μM, about 10 μM, about 9 μM, about 8 μM, about 7 μM, about 6 μM, about 5 μM, about 4 μM, about 3 μM, about 2 μM, about 1 μM, about 950 nM, about 900 nM, about 850 nM, about 800 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 50 nM, about 10 nM, about 5 nM, or about 1 nM. Compounds of formula (I) may activate NAPE-PLD with an AC₅₀ of less than 30 μM, less than 29 μM, less than 28 μM, less than 27 μM, less than 26 μM, less than 25 μM, less than 24 μM, less than 23 μM, less than 22 μM, less than 21 μM, less than 20 μM, less than 19 μM, less than 18 μM, less than 17 μM, less than 16 μM, less than 15 μM, less than 14 μM, less than 13 μM, less than 12 μM, less than 11 μM, less than 10 μM, less than 9 μM, less than 8 μM, less than 7 μM, less than 6 μM, less than 5 μM, less than 4 μM, less than 3 μM, less than 2 μM, less than 1 μM, less than 950 nM, less than 900 nM, less than 850 nM, less than 800 nM, less than 850 nM, less than 800 nM, less than 750 nM, less than 700 nM, less than 650 nM, less than 600 nM, less than 550 nM, less than 500 nM, less than 450 nM, less than 400 nM, less than 350 nM, less than 300 nM, less than 250 nM, less than 200 nM, less than 150 nM, less than 100 nM, less than 50 nM, less than 10 nM, less than 5 nM, or less than 1 nM.

### Pharmaceutical Salts

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides, and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### General Synthesis of Compounds of Formula (I)

Compounds of formula (I) may be prepared by synthetic processes. Abbreviations which have been used in the descriptions of the Schemes that follow are:
DCM is dichloromethane;
DIPEA is N,N-diisopropylethylamine;
DMF is dimethylformamide;
HATU is N-[(dimethylamino)-1H-1,2,3-triazolo-[4,5-b]pyridin-1-ylmethylene]-N-methylmethanaminium hexafluorophosphate N-oxide; and
TFA is trifluoro acetic acid.

Compounds of formula (I) or any of its subformulas may be synthesized as shown in the following schemes.

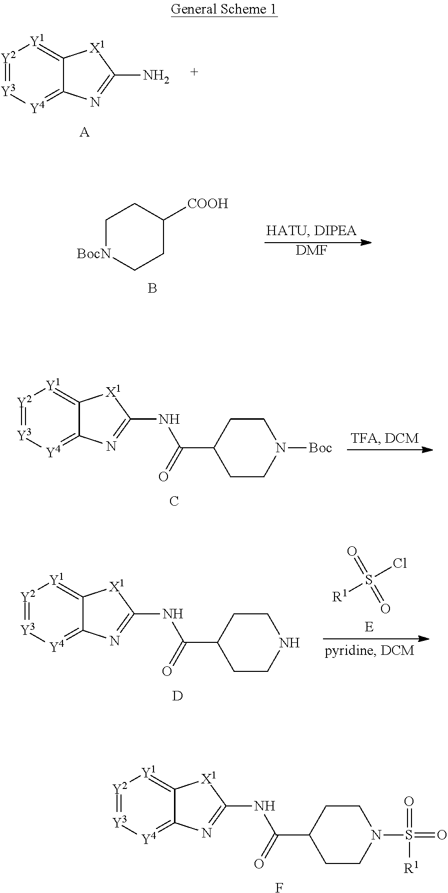

As shown in General Scheme 1, compounds of formula A may be subjected to peptide coupling conditions, wherein compound A is reacted with HATU and a compound of formula B under suitable basic conditions to form an intermediate compound of formula C. Subsequently, compounds of formula C may be subjected to suitable Boc-deprotection conditions to form an intermediate compound of formula D. Intermediate compounds of formula D may be reacted with a sulfonyl chloride compound of formula E under suitable conditions to produce a compound of formula F.

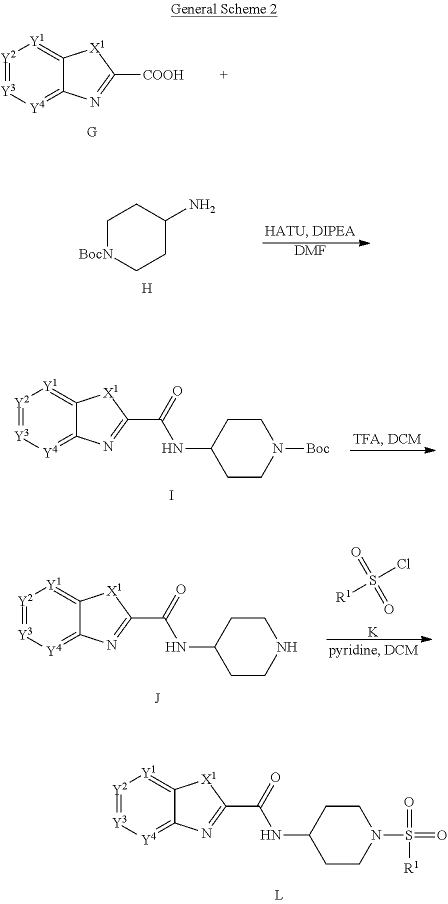

Alternatively, as shown in General Scheme 2, compounds of formula G may be subjected to peptide coupling conditions, wherein a compound of formula G is reacted with HATU and a compound of formula H under suitable basic conditions to form an intermediate compound of formula I. Subsequently, intermediate compounds of formula I may be subjected to suitable Boc-deprotection conditions to form an intermediate compound of formula J. Intermediate compounds of formula J may be reacted with a sulfonyl chloride compound of formula K under suitable conditions to produce a compound of formula L.

Peptide coupling conditions suitable for use in the processes of General Schemes 1 and 2 are well known in the art. Suitable peptide coupling conditions include those generally outlined in General Schemes 1 and 2, and as described in the Examples herein.

The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in Vogel's Textbook of Practical Organic Chemistry”, 5^{th} ed. (1989), by Furniss, Hannaford, Smith, and Tatchell, Longman Scientific & Technical, Essex CM20 2JE, England.

A disclosed compound may have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Optimum reaction conditions and reaction times for each individual step can vary depending on the particular reactants employed and substituents present in the reactants used. Specific procedures are provided in the Examples section. Reactions can be worked up in the conventional manner, e.g., by eliminating the solvent from the residue and further purified according to methodologies generally known in the art such as, but not limited to, crystallization, distillation, extraction, trituration, and chromatography. Unless otherwise described, the starting materials and reagents are either commercially available or can be prepared by one skilled in the art from commercially available materials using methods described in the chemical literature.

Starting materials, if not commercially available, can be prepared by procedures selected from standard organic chemical techniques, techniques that are analogous to the synthesis of known, structurally similar compounds, or techniques that are analogous to the above-described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th} ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization, or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

### Pharmaceutical Compositions

The disclosed compounds may be incorporated into pharmaceutical compositions suitable for administration to a subject (such as a patient, which may be a human or non-human). The pharmaceutical compositions may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention [e.g., a compound of formula (I)] are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their physiologically acceptable salts and solvates may be formulated for administration by, for example, solid dosing, eyedrop, in a topical oil-based formulation, injection, inhalation (either through the mouth or the nose), implants, or oral, buccal, parenteral, or rectal administration. Techniques and formulations may generally be found in “Remington's Pharmaceutical Sciences”, (Meade Publishing Co., Easton, Pa.). Therapeutic compositions must typically be sterile and stable under the conditions of manufacture and storage.

The route by which the disclosed compounds are administered, and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions. Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmelose, crospovidone, sodium carboxymethyl starch, sodium starch glycolate, clays, and ion exchange resins. The amount of disintegrant(s) in a systemic or topical composition is typically about 0.1 to about 10%. Suitable colorants include a colorant such as an FD&C dye. When used, the amount of colorant in a systemic or topical composition is typically about 0.005 to about 0.1%. Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%. Suitable antioxidants include butylated hydroxyanisole (“BHA”), butylated hydroxytoluene (“BHT”), and vitamin E. The amount of antioxidant(s) in a systemic or topical composition is typically about 0.1 to about 5%. Suitable preservatives include benzalkonium chloride, methyl paraben and sodium benzoate. The amount of preservative(s) in a systemic or topical composition is typically about 0.01 to about 5%. Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%. Suitable suspending agents include AVICEL RC-591 (from FMC Corporation of Philadelphia, PA) and sodium alginate.

The amount of suspending agent(s) in a systemic or topical composition is typically about 1 to about 8%. Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of active [e.g., compound of formula (I)] and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose, and cellulose. Specific binders include starch, gelatin, and sucrose. Specific disintegrants include alginic acid and croscarmelose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound [e.g., a compound of formula (I)], and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention. Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT coatings (available from Rohm & Haas G.M.B.H. of Darmstadt, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound (e.g., a compound of formula (I)), and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the medicament. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2^{nd} ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols. The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%. The amount of thickener(s) in a topical composition is typically about 0% to about 95%. Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified Montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%. The amount of fragrance in a topical composition is typically about 0% to about 0.5%, particularly, about 0.001% to about 0.1%. Suitable pH adjusting additives include HCl or NaOH in amounts sufficient to adjust the pH of a topical pharmaceutical composition.

### Methods of Treatment

The disclosed compounds and compositions may be used in methods for treatment of NAPE-PLD related medical diseases and/or disorders. The methods of treatment may comprise administering to a subject in need of such treatment a composition comprising a therapeutically effective amount of the compound of formula (I).

The compositions can be administered to a subject in need thereof to modulate NAPE-PLD, for a variety of diverse biological processes. The present disclosure is directed to methods for administering the composition to activate NAPE-PLD, an enzyme that plays a role in regulating metabolism and inflammation.

The compositions may be useful for treating and preventing certain diseases and disorders in humans and animals relating to NAPE-PLD dysfunction. Representative metabolic diseases and disorders include pre-diabetes, diabetes (type I or type II), metabolic syndrome, obesity, non-alcoholic fatty liver disease (NAFLD), non-alcoholic steatohepatitis (NASH), diabetic dyslipidemia, hyperlipidemia, hypertension, hypertriglyceridemia, hyperfattyacidemia, and hyperinsulinemia. The metabolic disorder may also include a histopathological change associated with chronic or acute hyperglycemia (e.g., degeneration of pancreas (beta-cell destruction), kidney tubule calcification, degeneration of liver, eye damage (diabetic retinopathy), diabetic foot, ulcerations in mucosa such as mouth and gums, excess bleeding, delayed blood coagulation, or wound healing). In some instances, the metabolic disease or disorder is obesity, type 2 diabetes, hyperlipidemia, non-alcoholic fatty liver disease, atherosclerosis, hypertriglyceridemia, or hypertension. In some instances, the metabolic disease or disorder is a non-healing wound, a chronic ulcer of the leg or foot, cellulitis or abscess of the leg, or gangrene.

Treatment or prevention of such diseases and disorders can be effected by activating or modulating NAPE-PLD in a subject, by administering a compound of formula (I) or composition disclosed herein, either alone or in combination with an appropriate ancillary agent as part of the therapeutic regimen to the subject in need thereof. In some instances, the ancillary agent is selected from an antidiabetic agent (e.g., metformin, glyburide, glimepiride, glipyride, glipizide, chlorpropamide, gliclazide, acarbose, miglitol, pioglitazone, troglitazone, dapagliflozin, rosiglitazone, insulin, GI-262570, isaglitazone, JTT-501, NN-2344, L895645, YM-440, R-119702, A39677, repaglinide, nateglinide, KAD1129, APR-H039242, GW-409544, KRP297, AC2993, Exendin-4, LY307161, NN2211 or LY315902), an anti-obesity agent (e.g., Orlistat, ATL-962, A39677, L750355, CP331648, sibutramine, topiramate, axokine, dexamphetamine, phentermine, phenylpropanolamine, famoxin, or mazindol) or a lipid-modulating agent (e.g., pravastatin, lovastatin, simvastatin, atorvastatin, cerivastatin, fluvastatin, nisvastatin, ZD-4522, fenofibrate, gemfibrozil, clofibrate, implitapide, CP-529,414, avasimibe, TS-962, MD-700, or LY295427).

### Adiposity, Hypertriglyceridemia, and Hepatic Steatosis

N-acyl-phosphatidylethanolamine hydrolyzing phospholipase D (NAPE-PLD) is a beta-lactamase fold zinc metallohydrolase that catalyzes the biosynthesis of N-acyl-ethanolamides (NAEs), such as N-oleoyl-ethanolamide (OEA) and N-palmitoyl-ethanolamide (PEA), by hydrolyzing appropriate precursor N-acyl-phosphatidylethanolamines (NAPEs). NAEs such as OEA and PEA exert pleiotropic effects against metabolic disease. OEA is rapidly biosynthesized in the intestinal tract in response to food intake and promotes satiety, fatty acid oxidation, and glucose-stimulated insulin secretion. Administering OEA to rodents fed a high-fat diet reduces food intake, fat accumulation, hyperglycemia, hyperlipidemia, inflammation, and hepatic steatosis. Like OEA, PEA is also biosynthesized in many peripheral tissues and exerts significant anti-inflammatory effects including inhibiting leukocyte chemotaxis to inflammatory stimuli and mast cell activation, and enhances the efferocytotic capacity of macrophages, which is essential for the resolution of inflammation. Administering PEA to rodents inhibits inflammation induced by various stimuli and reduces hypertriglyceridemia, and atherosclerotic lesion area and necrosis in atherosclerosis-prone mice fed a Western Diet.

Increasing intestinal NAPE-PLD expression via an adenoviral vector increased intestinal OEA and PEA levels and reduced food intake compared to the control vector. In contrast, high-fat diets markedly reduce NAPE-PLD expression and OEA and PEA levels in many tissues including intestine, aorta, spleen, and bone marrow. Mimicking this by selective deletion of intestinal NAPE-PLD reduced intestinal OEA and PEA levels and increased adiposity, hypertriglyceridemia, and hepatic steatosis. The selective deletion of hepatocyte NAPE-PLD resulted in hepatic steatosis and increased body fat. The selective deletion of adipocyte NAPE-PLD increased adiposity and hyperglycemia and prevented cold-induced adipocyte browning.

### Non-Healing Wounds

Reduced NAPE-PLD expression is also associated with wound ulceration. Chronic diabetic wounds are characterized by abnormal persistence of M1 macrophages that hinder wound healing. During normal wound healing, there is a transition from ˜85% M1 macrophages initially to 15-20% one week after injury. In contrast, nonhealing diabetic wounds fail to shift to M2, so that M1 macrophages comprise ˜80% of chronic wound margins. Macrophages in chronic wounds have been reported to have reduced efferocytosis capacity and accumulation of apoptotic neutrophils promote a strong inflammatory environment. Bioinformatic analysis has revealed that humans with genetically encoded reduced NAPE-PLD expression have markedly increased risk for foot and skin ulcers, a major complication of diabetes. Further, preliminary studies have shown that NAPE-PLD deletion in bone marrow-derived macrophage reduced their efferocytotic capacity.

It will be apparent to one of ordinary skill in the relevant art that suitable modifications and adaptations to the compositions, formulations, methods, processes, apparata, assemblies, and applications described herein can be made without departing from the scope of any embodiments or aspects thereof. The compositions, apparata, assemblies, and methods provided are exemplary and are not intended to limit the scope of any of the disclosed embodiments. All the various embodiments, aspects, and options disclosed herein can be combined in any variations or iterations. The scope of the compositions, formulations, methods, apparata, assemblies, and processes described herein include all actual or potential combinations of embodiments, aspects, options, examples, and preferences described herein. The compositions, formulations, apparata, assemblies, or methods described herein may omit any component or step, substitute any component or step disclosed herein, or include any component or step disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. All patents and publications cited herein are incorporated by reference herein for the specific teachings thereof.

Various embodiments and aspects of the inventions described herein are summarized by the following clauses:

Clause 1. A compound of formula (I):

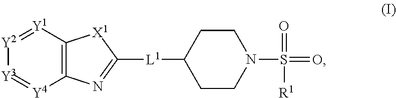

or a pharmaceutically acceptable salt thereof,
wherein:
X¹ is S or O;
Y¹ and Y³ are each C—R² and Y² and Y⁴ are each C—H, or
Y¹ and Y³ are each C—H and Y² and Y⁴ are each C—R²;
L¹ is

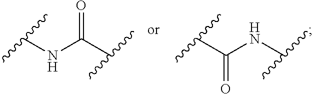 [SMILES: *NC(*)=O ; *NC(*)=O]

R¹ is G¹, —CH₂-G¹, or C₁₋₄alkyl;
R², at each occurrence, is independently C₁₋₄alkyl, C₁₋₄haloalkyl, halogen, C₃₋₄cycloalkyl, —OC₁₋₄alkyl, —OC₁₋₂haloalkyl, or —OC₃₋₄cycloalkyl;
G¹ is a phenyl, a 5- to 9-membered heteroaryl containing 1-3 heteroatoms, a 3- to 7-membered carbocycle, or a 4- to 6-membered heterocycle containing 1-2 heteroatoms, wherein G¹ is optionally substituted with 1 to 3 substituents, each independently R¹¹ or —C₁₋₂alkylene-R¹¹;
R¹¹ is C₁₋₄alkyl, C₁₋₄haloalkyl, C₃₋₄cycloalkyl, halogen, cyano, —OR^{11a}, —N(R^{11a})₂, —NR^{11a}C(O)R^{11b}, —C(O) R^{11b}, —CO₂R^{11b}, or —SO₂R^{11b};
R^{11a}, at each occurrence, are each independently hydrogen, C₁₋₄alkyl, C₁₋₄haloalkyl, or C₃₋₄cycloalkyl; and
R^{11b}, at each occurrence, is independently C₁₋₄alkyl, C₁₋₄haloalkyl, or C₃₋₄cycloalkyl;
with the proviso that the compound is not a compound of formula (I-a)

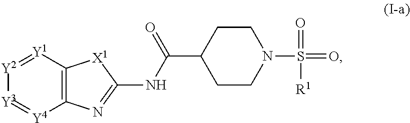

wherein:
i. X¹ is S,
   Y¹ and Y³ are each C—CH₃, and Y² and Y⁴ are each C—H, or
   Y¹ and Y³ are each C—H, and Y² and Y⁴ are each C—CH₃, Y² and Y⁴ are each C—F, or Y² is C—Cl and Y⁴ is C—CH₃;
   R¹ is methyl,

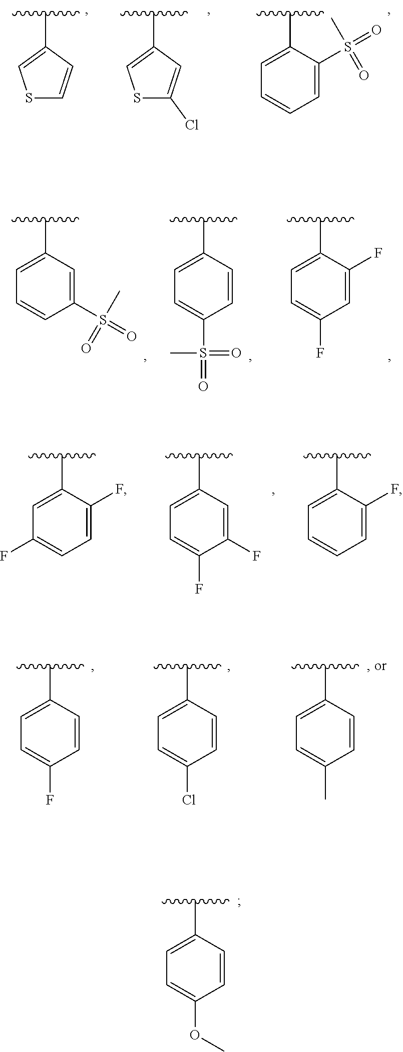

or wherein
ii. X¹ is S,
   Y¹ and Y³ are each C—H, and Y² and Y⁴ are each C—CH₃, and
   R¹ is ethyl, n-butyl,

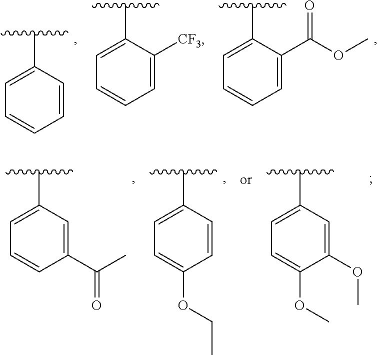

or wherein
iii. X¹ is S, Y¹ and Y³ are each C—H, Y² and Y⁴ are each C—F, and R¹ is

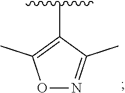

or wherein
iv. X¹ is O, Y¹ and Y³ are each C—CH₃, Y² and Y⁴ are each C—H, and R¹ is methyl.

Clause 2. The compound of clause 1, or a pharmaceutically acceptable salt thereof, wherein X¹ is S.

Clause 3. The compound of clause 1 or clause 2, or a pharmaceutically acceptable salt thereof, wherein Y¹ and Y³ are each C—C₁₋₄alkyl and Y² and Y⁴ are each C—H, or, where Y¹ and Y³ are each C—H and Y² and Y⁴ are each C—C₁₋₄alkyl.

Clause 4. The compound of any one of clauses 1-3, or a pharmaceutically acceptable salt thereof, wherein Y¹ and Y³ are each C—CH₃, and Y² and Y⁴ are each C—H.

Clause 5. The compound of any one of clauses 1-3, or a pharmaceutically acceptable salt thereof, wherein Y¹ and Y³ are each C—H, and Y² and Y⁴ are each C—CH₃.

Clause 6. The compound of any one of clauses 1-5, or a pharmaceutically acceptable salt thereof, wherein L¹ is

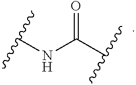 [SMILES: *NC(*)=O]

Clause 7. The compound of any one of clauses 1-7, or a pharmaceutically acceptable salt thereof, wherein R¹ is G¹.

Clause 8. The compound of clause 8, or a pharmaceutically acceptable salt thereof, wherein G¹ is the optionally substituted phenyl.

Clause 9. The compound of clause 9, or a pharmaceutically acceptable salt thereof, wherein G¹ is

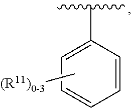

wherein R¹¹, at each occurrence, is independently halogen, cyano, C₁₋₄alkyl, C₁₋₂haloalkyl, —OC₁₋₄alkyl, or —OC₁₋₂haloalkyl.

Clause 10. The compound of clause 10, or a pharmaceutically acceptable salt thereof, wherein G¹ is

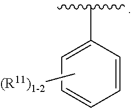

Clause 11. The compound of clause 11, or a pharmaceutically acceptable salt thereof, wherein G¹ is

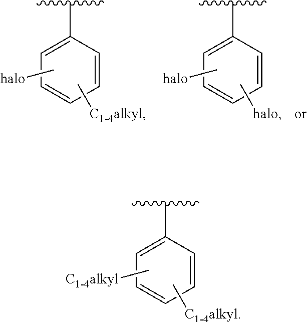

Clause 12. The compound of clause 12, or a pharmaceutically acceptable salt thereof, wherein G¹ is

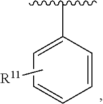

where R¹¹ is halogen, cyano, C₁₋₄alkyl, C₁₋₂haloalkyl, —OC₁₋₄alkyl, or —OC₁₋₂haloalkyl.

Clause 13. The compound of clause 8, or a pharmaceutically acceptable salt thereof, wherein G¹ is the optionally substituted 5- to 9-membered heteroaryl.

Clause 14. The compound of clause 14, or a pharmaceutically acceptable salt thereof, wherein the ring system of the optionally substituted 5- to 9-membered heteroaryl is a pyridinyl, a pyrimidinyl, a pyrazolyl, or a benzo[c][1,2,5]oxadiazolyl.

Clause 15. The compound of clause 15, or a pharmaceutically acceptable salt thereof, wherein R¹ is

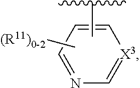

wherein X³ is C—H, or N, and R¹¹, at each occurrence, is independently halogen, cyano, C₁₋₄alkyl, C₁₋₂haloalkyl, —OC₁₋₄alkyl, or —OC₁₋₂haloalkyl.

Clause 16. The compound of clause 16, or a pharmaceutically acceptable salt thereof, wherein R¹ is

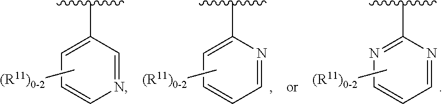

Clause 17. The compound of clause 17, or a pharmaceutically acceptable salt thereof, wherein R¹ is

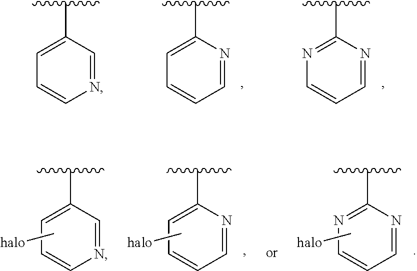

Clause 18. The compound of any one of any one of clauses 1-6, or a pharmaceutically acceptable salt thereof, wherein R¹ is —CH₂-G¹.

Clause 19. The compound of clause 8 or clause 19, or a pharmaceutically acceptable salt thereof, wherein G¹ is the optionally substituted 3- to 7-membered carbocycle.

Clause 20. The compound of clause 20, or a pharmaceutically acceptable salt thereof, wherein the ring system of the optionally substituted 3- to 7-membered carbocycle is a 3- or 6-membered carbocycle.

Clause 21. The compound of clause 21, or a pharmaceutically acceptable salt thereof, wherein R¹ is

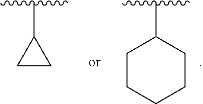

Clause 22. The compound of any one of clauses 1-6, or a pharmaceutically acceptable salt thereof, wherein R¹ is C₁₋₄alkyl.

Clause 23. The compound of clause 23, or a pharmaceutically acceptable salt thereof, wherein R¹ is methyl.

Clause 24. The compound of clause 1, wherein the compound is
1-((3-chloro-4-fluorophenyl)sulfonyl)-N-(5,7-dimethylbenzo[d]thiazol-2-yl)piperidine-4-carboxamide;
N-(4,6-dimethylbenzo[d]thiazol-2-yl)-1-((4-fluoro-2-methylphenyl)sulfonyl)piperidine-4-carboxamide;
N-(4,6-dimethylbenzo[d]thiazol-2-yl)-1-(pyridin-3-ylsulfonyl)piperidine-4-carboxamide;
N-(4,6-dimethylbenzo[d]thiazol-2-yl)-1-((1-methyl-1H-pyrazol-4-yl)sulfonyl)piperidine-4-carboxamide;
1-((2-chlorophenyl)sulfonyl)-N-(4,6-dimethylbenzo[d]thiazol-2-yl)piperidine-4-carboxamide;
N-(4,6-dimethylbenzo[d]thiazol-2-yl)-1-(o-tolylsulfonyl)piperidine-4-carboxamide;
N-(4,6-dimethylbenzo[d]thiazol-2-yl)-1-((3-fluorophenyl)sulfonyl)piperidine-4-carboxamide;
1-((3-chlorophenyl)sulfonyl)-N-(4,6-dimethylbenzo[d]thiazol-2-yl)piperidine-4-carboxamide;
N-(4,6-dimethylbenzo[d]thiazol-2-yl)-1-(m-tolylsulfonyl)piperidine-4-carboxamide;
1-((4-cyanophenyl)sulfonyl)-N-(4,6-dimethylbenzo[d]thiazol-2-yl)piperidine-4-carboxamide;
N-(4,6-dimethylbenzo[d]thiazol-2-yl)-1-((4-(trifluoromethoxy)phenyl)sulfonyl)piperidine-4-carboxamide;
1-((2-chloro-4-fluorophenyl)sulfonyl)-N-(4,6-dimethylbenzo[d]thiazol-2-yl)piperidine-4-carboxamide;
N-(4,6-dimethylbenzo[d]thiazol-2-yl)-1-((4-fluoro-2-methylphenyl)sulfonyl)piperidine-4-carboxamide;
N-(4,6-dimethylbenzo[d]thiazol-2-yl)-1-((6-fluoropyridin-3-yl)sulfonyl)piperidine-4-carboxamide;
N-(4,6-dimethylbenzo[d]thiazol-2-yl)-1-((5-fluoropyridin-2-yl)sulfonyl)piperidine-4-carboxamide;
N-(4,6-dimethylbenzo[d]thiazol-2-yl)-1-((5-fluoropyrimidin-2-yl)sulfonyl)piperidine-4-carboxamide;
N-(4,6-dimethylbenzo[d]thiazol-2-yl)-1-((2-fluoropyridin-3-yl)sulfonyl)piperidine-4-carboxamide;
N-(4,6-dimethylbenzo[d]thiazol-2-yl)-1-((3-fluoropyridin-2-yl)sulfonyl)piperidine-4-carboxamide;
N-(4,6-dimethylbenzo[d]thiazol-2-yl)-1-((3-fluoro-4-methylphenyl)sulfonyl)piperidine-4-carboxamide;
1-(cyclohexylsulfonyl)-N-(4,6-dimethylbenzo[d]thiazol-2-yl)piperidine-4-carboxamide;
1-((3,4-dichlorophenyl)sulfonyl)-N-(4,6-dimethylbenzo[d]thiazol-2-yl)piperidine-4-carboxamide;
1-((2,4-difluorobenzyl)sulfonyl)-N-(4,6-dimethylbenzo[d]thiazol-2-yl)piperidine-4-carboxamide;
1-(cyclopropylsulfonyl)-N-(4,6-dimethylbenzo[d]thiazol-2-yl)piperidine-4-carboxamide;
1-((cyclopropylmethyl)sulfonyl)-N-(4,6-dimethylbenzo[d]thiazol-2-yl)piperidine-4-carboxamide;
1-(benzo[c][1,2,5]oxadiazol-4-ylsulfonyl)-N-(4,6-dimethylbenzo[d]thiazol-2-yl)piperidine-4-carboxamide;
1-((3-chloro-4-fluorophenyl)sulfonyl)-N-(5,7-dimethylbenzo[d]thiazol-2-yl)piperidine-4-carboxamide;
N-(5,7-dimethylbenzo[d]thiazol-2-yl)-1-((4-fluoro-2-methylphenyl)sulfonyl)piperidine-4-carboxamide;
N-(5,7-dimethylbenzo[d]thiazol-2-yl)-1-(pyridin-3-ylsulfonyl)piperidine-4-carboxamide;
N-(5,7-dimethylbenzo[d]thiazol-2-yl)-1-((1-methyl-1H-pyrazol-4-yl)sulfonyl)piperidine-4-carboxamide;
1-((2-chlorophenyl)sulfonyl)-N-(5,7-dimethylbenzo[d]thiazol-2-yl)piperidine-4-carboxamide;
N-(5,7-dimethylbenzo[d]thiazol-2-yl)-1-(o-tolylsulfonyl)piperidine-4-carboxamide;
N-(5,7-dimethylbenzo[d]thiazol-2-yl)-1-((3-fluorophenyl)sulfonyl)piperidine-4-carboxamide;
1-((3-chlorophenyl)sulfonyl)-N-(5,7-dimethylbenzo[d]thiazol-2-yl)piperidine-4-carboxamide;
N-(5,7-dimethylbenzo[d]thiazol-2-yl)-1-(m-tolylsulfonyl)piperidine-4-carboxamide;
1-((4-cyanophenyl)sulfonyl)-N-(5,7-dimethylbenzo[d]thiazol-2-yl)piperidine-4-carboxamide;
N-(5,7-dimethylbenzo[d]thiazol-2-yl)-1-((4-(trifluoromethoxy)phenyl)sulfonyl)piperidine-4-carboxamide;
1-((2-chloro-4-fluorophenyl)sulfonyl)-N-(5,7-dimethylbenzo[d]thiazol-2-yl)piperidine-4-carboxamide;
N-(5,7-dimethylbenzo[d]thiazol-2-yl)-1-((4-fluoro-2-methylphenyl)sulfonyl)piperidine-4-carboxamide;
N-(5,7-dimethylbenzo[d]thiazol-2-yl)-1-((6-fluoropyridin-3-yl)sulfonyl)piperidine-4-carboxamide;
N-(5,7-dimethylbenzo[d]thiazol-2-yl)-1-((5-fluoropyridin-2-yl)sulfonyl)piperidine-4-carboxamide;
N-(5,7-dimethylbenzo[d]thiazol-2-yl)-1-((5-fluoropyrimidin-2-yl)sulfonyl)piperidine-4-carboxamide;
N-(5,7-dimethylbenzo[d]thiazol-2-yl)-1-((2-fluoropyridin-3-yl)sulfonyl)piperidine-4-carboxamide;
N-(5,7-dimethylbenzo[d]thiazol-2-yl)-1-((3-fluoropyridin-2-yl)sulfonyl)piperidine-4-carboxamide;
N-(5,7-dimethylbenzo[d]thiazol-2-yl)-1-((3-fluoro-4-methylphenyl)sulfonyl)piperidine-4-carboxamide;
1-(cyclohexylsulfonyl)-N-(5,7-dimethylbenzo[d]thiazol-2-yl)piperidine-4-carboxamide;
1-((3,4-dichlorophenyl)sulfonyl)-N-(5,7-dimethylbenzo[d]thiazol-2-yl)piperidine-4-carboxamide;
1-((2,4-difluorobenzyl)sulfonyl)-N-(5,7-dimethylbenzo[d]thiazol-2-yl)piperidine-4-carboxamide;
1-(cyclopropylsulfonyl)-N-(5,7-dimethylbenzo[d]thiazol-2-yl)piperidine-4-carboxamide;
1-((cyclopropylmethyl)sulfonyl)-N-(5,7-dimethylbenzo[d]thiazol-2-yl)piperidine-4-carboxamide; or
1-(benzo[c][1,2,5]oxadiazol-4-ylsulfonyl)-N-(5,7-dimethylbenzo[d]thiazol-2-yl)piperidine-4-carboxamide.

Clause 25. A pharmaceutical composition comprising the compound of any one of clauses 1-24 and a pharmaceutically acceptable carrier.

Clause 26. A pharmaceutical composition comprising a compound of formula (I)

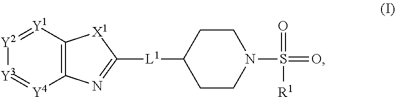

or a pharmaceutically acceptable salt thereof,
wherein:
X¹ is S or O;
Y¹, Y², Y³, and Y⁴ are each C—R² or C—H;
L¹ is

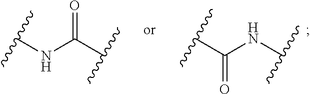 [SMILES: *NC(*)=O ; *NC(*)=O]

R¹ is G¹, —CH₂-G¹, or C₁₋₄alkyl;
R², at each occurrence, is independently C₁₋₄alkyl, C₁₋₄haloalkyl, halogen, C₃₋₄cycloalkyl, —OC₁₋₄alkyl, —OC₁₋₂haloalkyl, or —OC₃₋₄cycloalkyl;
G¹ is a phenyl, a 5- to 9-membered heteroaryl containing 1-3 heteroatoms, a 3- to 7-membered carbocycle, or a 4- to 6-membered heterocycle containing 1-2 heteroatoms, wherein G¹ is optionally substituted with 1 to 3 substituents, each independently R¹¹ or —C₁₋₂alkylene-R¹¹;
R¹¹ is C₁₋₄alkyl, C₁₋₄haloalkyl, C₃₋₄cycloalkyl, halogen, cyano, —OR^{11a}, —N(R^{11a})₂, —NR^{11a}C(O)R^{11b}, —C(O) R^{11b}, —CO₂R^{11b}, or —SO₂R^{11b};
R^{11a}, at each occurrence, are each independently hydrogen, C₁₋₄alkyl, C₁₋₄haloalkyl, or C₃₋₄cycloalkyl; and
R^{11b}, at each occurrence, is independently C₁₋₄alkyl, C₁₋₄haloalkyl, or C₃₋₄cycloalkyl;
and a pharmaceutically acceptable carrier,
with the proviso that the compound is not a compound of formula (I-b)

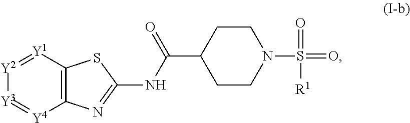

wherein:
i. Y¹, Y³, and Y⁴ are each C—H, Y² is C—CH₃, C—OCH₃, or C—OCH₂CH₃, and R¹ is

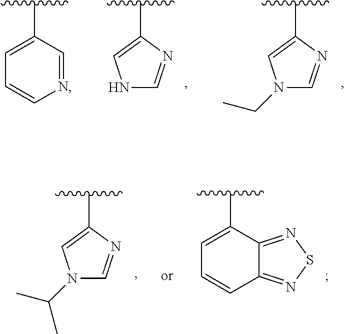

or wherein
ii. Y¹, Y³, and Y⁴ are each C—H, Y² is C—H, C—CH₃, C—OCH₃, or C—F, and R¹ is

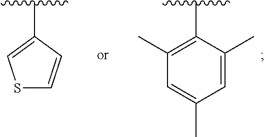

or wherein
iv. Y¹, Y³, and Y⁴ are each C—H, Y⁴ is C—CH₃, and R¹ is

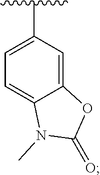 [SMILES: *c1ccc2c(c1)oc(=O)n2C]

or wherein
v. Y¹ and Y⁴ are each C—H, Y² and Y³ are each C—OH, and R¹ is

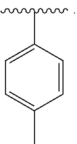 [SMILES: *c1ccc(C)cc1]

Clause 27. A method for treating a disease or disorder associated with metabolic dysfunction in a mammal, the method comprising administering to the mammal a therapeutically effective amount of the compound of any one of clauses 1-24, or pharmaceutically acceptable salt thereof, or the pharmaceutical composition of clause 25 or clause 26.

Clause 28. The method of clause 27, wherein the disease or disorder is associated with N-acyl phosphatidylethanolamine hydrolyzing phospholipase D (NAPE-PLD) dysfunction.

Clause 29. The method of clause 27 or clause 28, wherein the disease or disorder is obesity, type 2 diabetes, hyperlipidemia, non-alcoholic fatty liver disease, atherosclerosis, hypertriglyceridemia, or hypertension.

Clause 30. The method of clause 28 or clause 29, wherein the disease or disorder is a non-healing wound, a chronic ulcer of the leg or foot, cellulitis or abscess of the leg, or gangrene.

Clause 31. The pharmaceutical composition of clause 25 or clause 26, wherein the pharmaceutical composition is formulated for topical administration.

Clause 32. The compound of any one of clauses 1-24, or pharmaceutically acceptable salt thereof, or the pharmaceutical composition of clause 25 or clause 26, for use in the treatment of a disease or disorder associated with metabolic dysfunction in a mammal.

Clause 33. The use of the compound of any one of clauses 1-24, or pharmaceutically acceptable salt thereof, or the pharmaceutical composition of clause 25 or clause 26, for the preparation of a medicament for the treatment of a disease or disorder associated with metabolic dysfunction in a mammal.

The compounds and processes of the invention will be better understood by reference to the following examples, which are intended as an illustration of and not a limitation upon the scope of the invention.

### EXAMPLES

Abbreviations that may be used in the examples that follow are:
DCM is dichloromethane;
DIPEA is N,N-diisopropylethylamine;
DMF is dimethylformamide;
DMSO is dimethylsulfoxide;
EtOAc is ethyl acetate;
eq, eq., or equiv is equivalent(s);
HATU is N-[(dimethylamino)-1H-1,2,3-triazolo-[4,5-b]pyridin-1-ylmethylene]-N-methylmethanaminium hexafluorophosphate N-oxide;
Hex is hexanes;
h or hr is hour(s);
LCMS is liquid chromatography mass spectrometry;
MeOH is methanol;
min or min. is minute(s);
rt, RT, or r.t. is room temperature;
sat. is saturated;
s or see is second(s); and
TFA is trifluoro acetic acid.

### Example 1

### Chemical Synthesis and Characterization

### Purchased Compounds

VU484, VU485, VU486, VU488, VU539, VU542, VU517, VU534, VU533, VU575, VU601, VU605 (as shown in Table 4) were purchased from Life Chemicals.

VU542 and VU534 were also synthesized and characterized by the Vanderbilt Chemical Synthesis core as well as the remaining series of additional benzothiazole compounds as described below.

### Synthesis of Benzothiazole Phenylsulfonyl-Piperidine Carboxamides

### General Procedure

All non-aqueous reactions were performed in flame-dried or oven dried round-bottomed flasks under an atmosphere of argon. Stainless steel syringes or cannula were used to transfer air- and moisture-sensitive liquids. Reaction temperatures were controlled using a thermocouple thermometer and analog hotplate stirrer. Reactions were conducted at room temperature (approximately 23° C.) unless otherwise noted. Flash column chromatography was conducted using silica gel 230-400 mesh. Analytical thin-layer chromatography (TLC) was performed on E. Merck silica gel 60 F254 plates and visualized using UV, and potassium permanganate stain. Yields were reported as isolated, spectroscopically pure compounds.

### Materials

Solvents were obtained from either an MBraun MB-SPS solvent system or freshly distilled (tetrahydrofuran was distilled from sodium-benzophenone; toluene was distilled from calcium hydride and used immediately; dimethyl sulfoxide was distilled from calcium hydride and stored over 4 Å molecular sieves). Commercial reagents were used as received. The molarity of n-butyllithium solutions was determined by titration using diphenylacetic acid as an indicator (average of three determinations).

### Instrumentation

Semi-preparative reverse phase HPLC was conducted on a Waters HPLC system using a Phenomenex Luna 5 μm C18(2) 100A Axia 250×10.00 mm column or preparative reverse phase HPLC (Gilson) using a Phenomenex Luna column (100 Å, 50×21.20 mm, 5 μm C18) with UV/Vis detection. Infrared spectra were obtained as thin films on NaCl plates using a Thermo Electron IR100 series instrument and are reported in terms of frequency of absorption (cm⁻¹). ¹H NMR spectra were recorded on Bruker 400, 500, or 600 MHz spectrometers and are reported relative to deuterated solvent signals. Data for ¹H NMR spectra are reported as follows: chemical shift (δ ppm), multiplicity (s=singlet, d=doublet, t=triplet, q=quartet, p=pentet, m=multiplet, br=broad, app=apparent), coupling constants (Hz), and integration. ¹³C NMR spectra were recorded on Bruker 100, 125, or 150 MHz spectrometers and are reported relative to deuterated solvent signals. LC/MS was conducted and recorded on an Agilent Technologies 6130 Quadrupole instrument.

### Compound Preparation

### General Synthesis of Benzothiazole Phenylsulfonyl-Piperidine Carboxamides

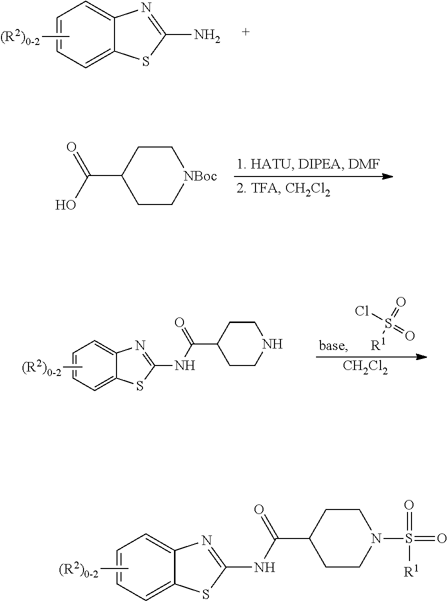

### General Amide Coupling

To a solution of N-Boc-piperidine-4-carboxylic acid (2 g, 13.3 mmol) and HATU (7.6 g, 19.9 mmol) in DMF (40 mL) at 0° C. was added diisopropylethylamine (6.9 mL, 40 mmol) dropwise. After 5 min a solution of a 2-aminobenzothiazole (13.3 mmol) in DMF (5 mL) was added dropwise. The reaction was allowed to warm to ambient temperature and stirred for 24 h. The resulting red-brown solution was quenched with water (20 mL) and extracted with ethyl acetate (3×50 mL). The organic extracts were combined and washed with water (20 mL) followed by brine (20 mL). The organic layer was dried (MgSO₄), filtered and concentrated in vacuo. The resulting residue was purified by column chromatography.

### General Deprotection

To a solution of solution of N-Boc-piperidine amide (5.5 mmol) in dichloromethane (20 mL) was added trifluoroacetic acid (1.25 mL, 16.6 mmol). The reaction was heated to reflux and stirred for 16 h. The mixture was cooled to room temperature and concentrated in vacuo. The resulting solid was taken up in a solution of DCM: MeOH (9:1) and neutralized by the addition of 1 M NaOH as judged by pH and the mixture extracted with dichloromethane (4×50 mL). The organic extracts were combined, washed with brine (10 mL), dried (MgSO₄), filtered, and concentrated in vacuo. The resulting tan solid was recrystallized from hot chloroform by slow cooling. Further product was achieved by layering of the supernatant with hexanes to give a crystalline solid.

### General Sulfonamide Formation

A vial (4 mL) equipped with a stir bar was charged with amine (1 equiv.) followed by addition of dichloromethane (to a concentration of 100 mM) and triethylamine (1.5 equiv.). A solution of arylsulfonyl chloride (1.2 equiv.) in dichloromethane was added to the reaction mixture at room temperature. The reaction mixture was maintained for 16 h and diluted with dichloromethane-methanol (9:1). The mixture was washed with saturated NaHCO₃(aq.) followed by water. The organic layer was dried (MgSO₄) and concentrated in vacuo. The residue was purified by recrystallization from hot chloroform.

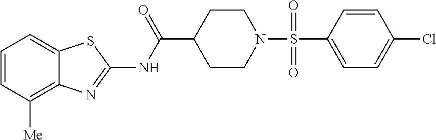 [SMILES: Cc1cccc2sc(NC(=O)C3CCN(S(=O)(=O)c4ccc(Cl)cc4)CC3)nc12]

### VU542 (Table 4, Entry 6)

Prepared from 4-chloro-benzenesulfonyl chloride (46 mg, 0.22 mmol) according to the general procedure described above. 42 mg, 51% of the product was obtained as an off-white crystalline solid: ¹H NMR (d₆-DMSO, 400 MHz): δ 7.79-7.72 (m, 5H), 7.22 (d, J=7.2 Hz, 1H), 7.28 (t, J=8.8 Hz, 1H), 4.07 (q, J=5.2 Hz, 1H), 3.68-3.65 (m, 2H), 2.54 (s, 3H), 2.35 (dt, d, J=11.6 Hz, 2.4 Hz, 2H), 1.98-1.90 (m, 2H), 1.65 (dq, J=13.2 Hz, 4.0 Hz, 2H); LCMS calc'd for C₂₀H₂₀ClN₃O₃S₂ [M+H]⁺: 449.9. measured 450.1.

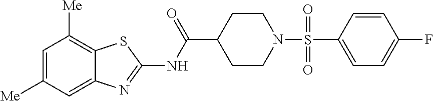 [SMILES: Cc1cc(C)c2sc(NC(=O)C3CCN(S(=O)(=O)c4ccc(F)cc4)CC3)nc2c1]

### VU534 (Table 4, Entry 8)

Prepared from 4-fluoro-benzenesulfonyl chloride (97 mg, 0.50 mmol) according to the general procedure described above. 94 mg, 84% of the product was obtained as an off-white crystalline solid: ¹H NMR (CDCl₃, 400 MHz): δ 7.70 (dd, J=9.2 Hz, 5.2 Hz, 1H), 7.69 (t, J=4.8 Hz, 1H), 7.28 (bs, 1H), 7.22 (t, J=8.8 Hz, 2H), 6.56 (s, 1H), 3.66 (dd, J=8.4 Hz, 3.2 Hz, 2H), 2.52 (s, 3H), 2.32-2.26 (m, 1H), 2.25 (s, 3H), 1.98 (dt, J=11.6 Hz, 2.4 Hz, 2H), 1.86 (dq, J=11.6 Hz, 4.0 Hz, 2H), 1.74 (dd, J=13.6 Hz, 3.2 Hz, 2H); ¹³C NMR (CDCl₃, 400 MHz): δ 172.6, 166.4, 163.9, 159.5, 147.2, 136.4, 132.3, 132.2, 131.7, 130.2, 130.1, 128.8, 126.2, 117.6, 116.4, 116.2, 44.9, 41.6, 27.6, 21.3, 20.5; LCMS calc'd for C₂₁H₂₂FN₃O₃S₂ [M+H]⁺: 447.5. measured 448.1.

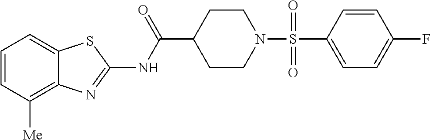 [SMILES: Cc1cccc2sc(NC(=O)C3CCN(S(=O)(=O)c4ccc(F)cc4)CC3)nc12]

### VU231 (Table 4, Entry 14)

Prepared from 4-fluoro-benzenesulfonyl chloride (97 mg, 0.50 mmol) according to the general procedure described above. 65 mg, 60% of the product was obtained as an off-white crystalline solid: ¹H NMR (CD₃OD, 400 MHz): δ 7.88 (dd, J=8.8 Hz, 4.8 Hz, 1H), 7.87 (t, J=4.8 Hz, 1H), 7.65 (d, J=7.6 Hz, 1H), 7.37 (dt, J=8.8 Hz, 2.0 Hz, 2H), 7.22 (d, J=6.4 Hz, 1H), 7.19 (q, J=7.6 Hz, 1H), 3.82 (dd, J=8.4 Hz, 3.2 Hz, 2H), 2.63 (s, 3H), 2.6-2.45 (m, 3H), 2.01 (dd, J=13.6 Hz, 3.2 Hz, 2H), 1.87 (dq, J=11.6 Hz, 4.0 Hz, 2H); LCMS calc'd for C₂₀H₂₀FN₃O₃S₂ [M+H]⁺: 433.5. measured 434.1.

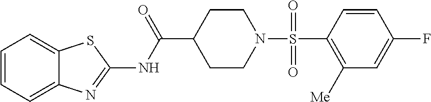 [SMILES: Cc1cc(F)ccc1S(=O)(=O)N1CCC(C(=O)Nc2nc3ccccc3s2)CC1]

### VU205 (Table 4, Entry 15)

Prepared from 4-fluoro-2-methylbenzenesulfonyl chloride (48 mg, 0.23 mmol) according to the general procedure described above. 53 mg, 64% of the product was obtained as a beige crystalline solid: ¹H NMR (d₆-DMSO, 400 MHz): δ 7.95 (d, J=8.1 Hz, 1H), 7.88 (dd, J=8.8 Hz, 5.9 Hz, 1H), 7.71 (d, J=8.0 Hz, 1H), 7.42 (t, J=7.8 Hz, 1H), 7.37 (dd, J=9.9 Hz, 2.7 Hz, 1H), 7.28 (m, 2H), 3.63 (d, J=12.5 Hz, 2H), 2.68 (m, 3H), 2.57 (s, 3H), 1.94 (d, J=13.6 Hz, 2H), 1.62 (qd, J=11.9 Hz, 3.6 Hz, 2H); ¹³C NMR (d₆-DMSO, 400 MHz): δ 173.9, 165.7, 163.2, 158.2, 148.9, 141.5, 133.1, 132.5, 131.8, 126.5, 123.9, 122.1, 120.9, 120.1, 119.9, 113.7, 75.1, 44.7, 41.0, 27.86, 20.6; LCMS calc'd for C₂₀H₂₀FN₃O₃S₂ [M+H]⁺: 433.5. measured 434.1.

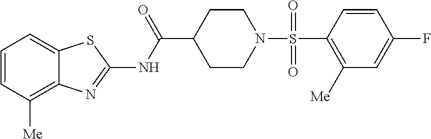 [SMILES: Cc1cc(F)ccc1S(=O)(=O)N1CCC(C(=O)Nc2nc3c(C)cccc3s2)CC1]

### VU212 (Table 4, Entry 16)

Prepared from 4-fluoro-2-methylbenzenesulfonyl chloride (48 mg, 0.23 mmol) according to the general procedure described above. 44 mg, 54% of the product was obtained as an off-white crystalline solid: ¹H NMR (d₆-DMSO, 400 MHz): δ 7.88 (dd, J=8.9 Hz, 5.9 Hz, 1H), 7.76 (d, J=7.7 Hz, 1H), 7.36 (dd, J=9.6 Hz, 2.5 Hz, 1H), 7.26 (td, J=8.4 Hz, 2.9 Hz, 1H), 7.23 (d, J=7.1 Hz, 1H), 7.17 (t, J=7.6 Hz, 1H), 3.63 (d, J=12.8 Hz, 2H), 2.66 (m, 3H), 2.57 (s, 3H), 2.54 (s, 3H), 1.93 (d, J=12.8 Hz, 2H), 1.62 (qd, J=12.6 Hz, 3.4 Hz, 2H); ¹³C NMR (d₆-DMSO, 400 MHz): δ 173.9, 157.4, 147.9, 141.5, 133.1, 132.5, 131.5, 130.1, 126.9, 123.8, 120.0, 119.4, 113.7, 74.9, 44.7, 41.0, 27.9, 20.6, 18.4; LCMS calc'd for C₂₁H₂₂FN₃O₃S₂ [M+H]⁺: 447.5. measured 448.1.

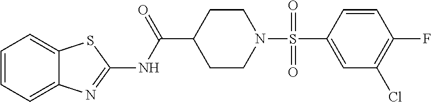 [SMILES: O=C(Nc1nc2ccccc2s1)C1CCN(S(=O)(=O)c2ccc(F)c(Cl)c2)CC1]

### VU233 (Table 4, Entry 17)

Prepared from 3-chloro-4-fluorobenzenesulfonyl chloride (53 mg, 0.23 mmol) according to the general procedure described above. 59 mg, 68% of the product was obtained as a white crystalline solid: ¹H NMR (d₆-DMSO, 400 MHz): δ 7.98 (dd, J=6.8 Hz, 2.2 Hz, 1H), 7.94 (d, J=7.9 Hz, 1H), 7.80 (ddd, J=8.7 Hz, 2.4 Hz, 2.2 Hz, 1H), 7.71 (m, 2H), 7.41 (t, J=7.8 Hz, 1H), 7.28 (t, J=7.8 Hz, 1H), 3.68 (d, J=11.9 Hz, 2H), 2.54 (tt, J=11.5 Hz, 3.8 Hz, 1H), 2.41 (td, J=11.8 Hz, 2.1 Hz, 2H), 1.95 (dd, J=13.4 Hz, 2.4 Hz, 2H), 1.65 (qd, J=12.3 Hz, 3.9 Hz, 2H); ¹³C NMR (d₆-DMSO, 400 MHz): δ 173.9, 159.0, 158.3, 148.9, 133.6, 131.8, 130.3, 129.3, 126.5, 123.9, 122.1, 121.6, 121.4, 120.8, 118.7, 118.5, 75.2, 45.7, 40.6, 27.6; LCMS calc'd for C₁₉H₁₇ClFN₃O₃S₂ [M+H]⁺: 453.9. measured 454.1.

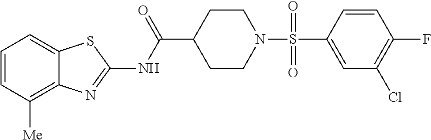 [SMILES: Cc1cccc2sc(NC(=O)C3CCN(S(=O)(=O)c4ccc(F)c(Cl)c4)CC3)nc12]

### VU209 (Table 4, Entry 18)

Prepared from 3-chloro-4-fluorobenzenesulfonyl chloride (53 mg, 0.23 mmol) according to the general procedure described above. 32 mg, 38% of the product was obtained as a white crystalline solid: ¹H NMR (d₆-DMSO, 400 MHz): δ 7.99 (dd, J=6.8 Hz, 2.2 Hz, 1H), 7.81 (m, 1H), 7.75 (d, J=7.7 Hz, 1H), 7.71 (t, J=8.9 Hz, 1H), 7.23 (d, J=6.9 Hz, 1H), 7.17 (t, J=7.8 Hz, 1H), 3.69 (d, J=11.9 Hz, 2H), 2.55 (s, 3H), 2.54 (m, 1H), 2.40 (t, J=12.0 Hz, 2H), 1.95 (d, J=12.6 Hz, 2H), 1.64 (qd, J=12.6 Hz, 3.4 Hz, 2H); ¹³C NMR (d₆-DMSO, 400 MHz): LCMS calc'd for C₂₀H₁₉ClFN₃O₃S₂ [M+H]⁺: 467.9. measured 468.1.

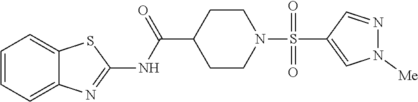 [SMILES: Cn1cc(S(=O)(=O)N2CCC(C(=O)Nc3nc4ccccc4s3)CC2)cn1]

### VU227 (Table 4, Entry 19)

Prepared from (1-methyl-1H-pyrazol-4-yl)sulfonyl chloride (41 mg, 0.23 mmol) according to the general procedure described above. 32 mg, 42% of the product was obtained as a white crystalline solid: ¹H NMR (d₆-DMSO, 400 MHz): δ 8.34 (s, 1H), 7.94 (d, J=7.8 Hz, 1H), 7.79 (s, 1H), 7.70 (d, J=7.7 Hz, 1H), 7.40 (t, J=7.7 Hz, 1H), 7.27 (t, J=7.7 Hz, 1H), 3.91 (s, 3H), 3.57 (d, J=11.6 Hz, 2H), 2.52 (m, 1H), 2.27 (td, J=11.8 Hz, 2.1 Hz, 2H), 1.97 (dd, J=13.4 Hz, 2.4 Hz, 2H), 1.69 (qd, J=12.3 Hz, 3.9 Hz, 2H); LCMS calc'd for C₁₇H₁₉N₅O₃S₂ [M+H]⁺: 405.5. measured 406.1.

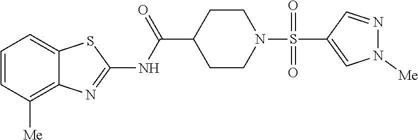 [SMILES: Cc1cccc2sc(NC(=O)C3CCN(S(=O)(=O)c4cnn(C)c4)CC3)nc12]

### VU210 (Table 4, Entry 20)

Prepared from (1-methyl-1H-pyrazol-4-yl)sulfonyl chloride (41 mg, 0.23 mmol) according to the general procedure described above. 43 mg, 56% of the product was obtained as a white crystalline solid: ¹H NMR (d₆-DMSO, 400 MHz): δ 8.34 (s, 1H), 7.80 (s, 1H), 7.75 (d, J=7.6 Hz, 1H), 7.23 (d, J=7.2 Hz, 1H), 7.17 (t, J=7.6 Hz, 1H), 3.91 (s, 3H), 3.56 (d, J=11.9 Hz, 2H), 2.54 (s, 3H), 2.51 (m, 1H), 2.25 (t, J=11.8 Hz, 2H), 1.96 (d, J=12.6 Hz, 2H), 1.68 (qd, J=12.6 Hz, 3.4 Hz, 2H); ¹³C NMR (d₆-DMSO, 400 MHz): δ 174.0, 138.9, 133.7, 131.5, 130.1, 126.9, 123.8, 119.4, 116.3, 75.0, 45.9, 40.7, 27.5, 18.4; LCMS calc'd for C₁₈H₂₁N₅O₃S₂ [M+H]⁺: 419.5. measured 420.2.

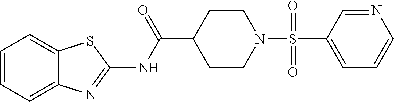 [SMILES: O=C(Nc1nc2ccccc2s1)C1CCN(S(=O)(=O)c2cccnc2)CC1]

### VU203 (Table 4, Entry 21)

Prepared from pyridine-3-sulfonyl chloride (41 mg, 0.23 mmol) according to the general procedure described above. 46 mg, 53% of the product was obtained as a white crystalline solid: ¹H NMR (d₆-DMSO, 400 MHz): δ 8.92 (d, J=2.1 Hz, 1H), 8.89 (dd, J=4.9 Hz, 1.5 Hz, 1H), 8.19 (dt, J=8.2 Hz, 2.1 Hz, 1H), 7.93 (d, J=7.8 Hz, 1H), 7.70 (m, 2H), 7.40 (t, J=7.7 Hz, 1H), 7.27 (t, J=7.7 Hz, 1H), 3.70 (d, J=11.9 Hz, 2H), 2.56 (tt, J=11.5 Hz, 3.8 Hz, 1H), 2.42 (td, J=11.8 Hz, 2.1 Hz, 2H), 1.95 (dd, J=13.4 Hz, 2.4 Hz, 2H), 1.63 (qd, J=12.3 Hz, 3.9 Hz, 2H); ¹³C NMR (de-DMSO, 400 MHz): δ 174.0, 158.5, 154.1, 148.9, 148.1, 135.9, 132.7, 131.8, 126.4, 124.9, 123.8, 122.0, 120.8, 75.0, 45.5, 40.6, 27.6; LCMS calc'd for C₁₈H₁₈N₄O₃S₂ [M+H]⁺: 402.5. measured 403.1.

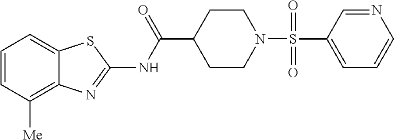 [SMILES: Cc1cccc2sc(NC(=O)C3CCN(S(=O)(=O)c4cccnc4)CC3)nc12]

### VU211 (Table 4, Entry 22)

Prepared from pyridine-3-sulfonyl chloride (41 mg, 0.23 mmol) according to the general procedure described above. 26 mg, 34% of the product was obtained as a beige crystalline solid: ¹H NMR (d₆-DMSO, 400 MHz): δ 8.92 (d, J=2.2 Hz, 1H), 8.90 (dd, J=4.9 Hz, 1.4 Hz, 1H), 8.18 (dt, J=8.0 Hz, 1.9 Hz, 1H), 7.73 (d, J=7.7 Hz, 1H), 7.70 (dd, J=7.8 Hz, 4.9 Hz, 1H), 7.22 (d, J=7.1 Hz, 1H), 7.17 (t, J=7.5 Hz, 1H), 3.71 (d, J=12.0 Hz, 2H), 2.55 (m, 1H), 2.54 (s, 3H), 2.42 (t, J=11.8 Hz, 2H), 1.94 (d, J=12.6 Hz, 2H), 1.64 (qd, J=12.6 Hz, 3.4 Hz, 2H); ¹³C NMR (d₆-DMSO, 400 MHz): δ 173.8, 157.4, 154.1, 148.1, 147.9, 136.0, 132.7, 131.5, 130.1, 126.9, 124.9, 123.8, 119.4, 45.5, 27.6, 18.4; LCMS calc'd for C₁₉H₂₀N₄O₃S₂ [M+H]⁺: 416.5. measured 417.1.

### Synthesis Example 1

### N-(5,7-Dimethylbenzo[d]thiazol-2-yl)-1-((4-fluorophenyl)sulfonyl)piperidine-4-carboxamide (VU0506534, Abbreviated as “VU534”)

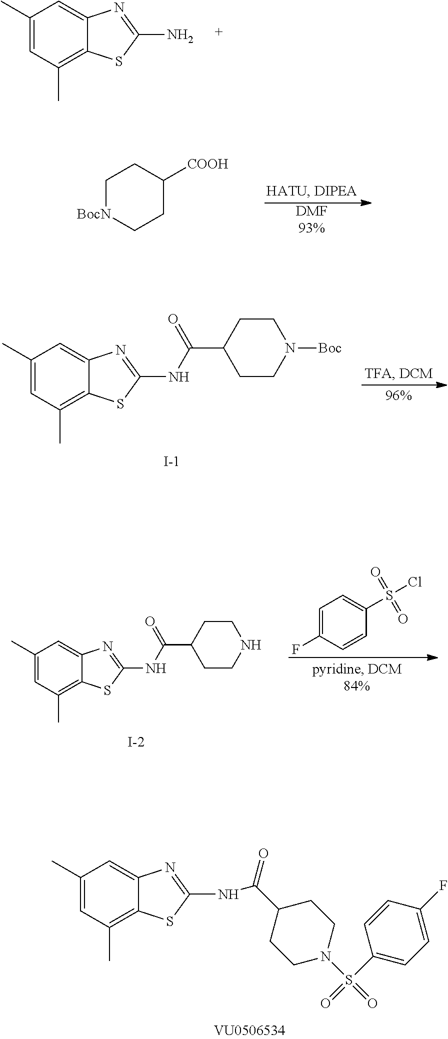

i. Preparation of tert-butyl 4-((5,7-dimethylbenzo[d]thiazol-2-yl) carbamoyl)piperidine-1-carboxylate (I-1). To a solution of 1-(tert-butoxycarbonyl)piperidine-4-carboxylic acid (0.386 g, 1.68 mmol) in DMF (5.6 mL) at 0° C. was added DIPEA (0.88 mL, 5.04 mmol) and HATU (0.96 g, 2.52 mmol). After stirring for 10 min at 0° C., 5,7-dimethylbenzo[d]thiazol-2-amine (0.3 g, 1.68 mmol) was added to reaction mixture. The reaction mixture was stirred for 5 h at room temperature, diluted with EtOAc (20 mL), and washed with water (20 mL×3). The combined organic layer was dried over MgSO₄, concentrated in vacuo, and purified on a Teledyne ISCO Combi-Flash system via normal phase chromatography on silica gel (0-40% gradient elution of EtOAc in hexanes) to afford I-1 as a white solid (0.61 g, 93%). LCMS calculated for C₂₀H₂₇N₃O₃S [M+H]⁺: 389.5 measured 390.5.

ii. Preparation of N-(5,7-dimethylbenzo[d]thiazol-2-yl)piperidine-4-carboxamide (I-2). To a solution of carbamate I-1 (0.61 g, 1.57 mmol) in dichloromethane (5.2 mL) was added TFA (0.72 mL, 9.41 mmol) at room temperature. The reaction mixture was heated at 38° C. for 3 h and then concentrated in vacuo. The residue was dissolved in chloroform (9 mL), basified with NaOH (1.0 M solution), and extracted with premixed solution of chloroform with 10% of MeOH (30 mL×3). The combined organic layer was dried over MgSO₄, concentrated in vacuo, and purified on a Teledyne ISCO Combi-Flash system via normal phase chromatography on silica gel (0-10% gradient elution of MeOH in dichloromethane) to afford I-2 as a white solid (0.44 g, 96%). LCMS calculated for C₁₅H₁₉N₃OS [M+H]⁺: 289.5 measured 290.3.

iii. Preparation of the title compound, N-(5,7-dimethylbenzo[d]thiazol-2-yl)-1-((4-fluorophenyl)sulfonyl)piperidine-4-carboxamide (VU0506534). To a solution of amine I-2 (73 mg, 0.25 mmol) in dichloromethane (2 mL) was added pyridine (81 μL, 1.0 mmol) and 4-fluorobenzenesulfonyl chloride (97.3 mg, 0.50 mmol) at 0° C. The reaction mixture was stirred at room temperature for 6 h, quenched with saturated NH₄Cl (10 mL), and extracted with DCM (10 mL×3). The combined organic layer was dried over MgSO₄, concentrated in vacuo, and purified on a Teledyne ISCO Combi-Flash system via normal phase chromatography on silica gel (0-50% gradient elution of EtOAc in hexanes) to afford the title compound as a white solid (94 mg, 84%). LCMS calculated for C₂₁H₂₂FN₃O₃S₂ [M+H]⁺: 447.5 measured 448.3. ¹H NMR (CDCl₃), 400 MHz): δ 7.70 (dd, J=9.2 Hz, 5.2 Hz, 1H), 7.69 (t, J=4.8 Hz, 1H), 7.28 (bs, 1H), 7.22 (t, J=8.8 Hz, 2H), 6.56 (s, 1H), 3.66 (dd, J=8.4 Hz, 3.2 Hz, 2H), 2.52 (s, 3H), 2.32-2.26 (m, 1H), 2.25 (s, 3H), 1.98 (dt, J=11.6 Hz, 2.4 Hz, 2H), 1.86 (dq, J=11.6 Hz, 4.0 Hz, 2H), 1.74 (dd, J=13.6 Hz, 3.2 Hz, 2H)

### Synthesis Example 2

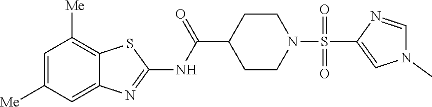 [SMILES: Cc1cc(C)c2sc(NC(=O)C3CCN(S(=O)(=O)c4cn(C)cn4)CC3)nc2c1]

### VU093368

N-(5-7-Dimethylbenzo[d]thiazol-2-yl)-1-((1-methyl-1H-imidazol-4-yl)sulfonyl)piperidine-4-carboxamide: In a manner similar to that described in Step iii of Example 1, N-(5,7-dimethylbenzo[d]thiazol-2-yl)piperidine-4-carboxamide TFA (50 mg, 0.17 mmol), DIPEA (0.06 mL, 0.34 mmol), 1-methyl-1H-pyrazole-4-sulfonyl chloride (46 mg, 0.26 mmol) and dichloromethane (3 mL) afforded the title compound (21 mg, 28%). LC=95% at 254 nm RT=1.0 min, MS=434 (m+1). ¹H NMR (CDCl₃, 400 MHz): δ 7.70 (s, 1H), 7.64 (s, 1H), 7.35 (S, 1H), 6.94 (s, 1H), 3.98 (s, 3H), 3.65 (mm, 2H), 2.53 (s, 3H), 2.34 (s, 3H), 2.15 (mm, 2H), 1.90 (mm, 4H),

### Synthesis Example 3

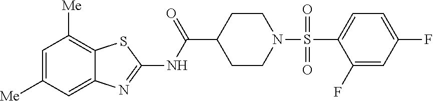 [SMILES: Cc1cc(C)c2sc(NC(=O)C3CCN(S(=O)(=O)c4ccc(F)cc4F)CC3)nc2c1]

### VU0943362

1-((2,4-Difluorophenyl)sulfonyl)-N-(5,7-dimethylbenzo[d]thiazol-2-yl)piperidine-4-carboxamide: In a manner similar to that described in Step iii of Example 1, N-(5,7-dimethylbenzo[d]thiazol-2-yl)piperidine-4-carboxamide (40 mg, 0.14 mmol), DIPEA (0.07 mL, 0.42 mmol), 2,4-difluorobenzenesulfonyl chloride (58 mg, 0.27 mmol) and dichloromethane (3.5 mL) afforded the title compound (32 mg, 49%). LC=99% at 254 nm RT=1.12 min, MS=466 (m+1).

### Synthesis Example 4

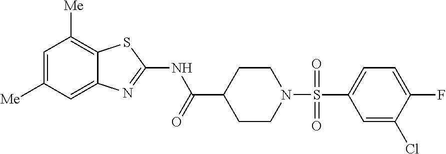 [SMILES: Cc1cc(C)c2sc(NC(=O)C3CCN(S(=O)(=O)c4ccc(F)c(Cl)c4)CC3)nc2c1]

### VU00943359

1-((3-Chloro-4-fluorophenyl)sulfonyl)-N-(5,7-dimethylbenzo[d]thiazol-2-yl)piperidine-4-carboxamide: In a manner similar to that described in Step iii of Example 1, N-(5,7-dimethylbenzo[d]thiazol-2-yl)piperidine-4-carboxamide (40 mg, 0.14 mmol), DIPEA (0.07 mL, 0.42 mmol), 3-chloro-4-fluorobenzenesulfonyl chloride (63 mg, 0.27 mmol) and dichloromethane (3.5 mL) afforded the title compound (30 mg, 45%). LC=99% at 254 nm RT=1.17 min, MS=482 (m+1). ¹H NMR (CDCl₃, 400 MHz): δ 7.80 (dd, J=2.07 Hz, J=2.07 Hz, 1H) 7.63 (mm, 1H), 7.35 (s, 1H), 7.31 (t, J=8.28 Hz, 1H), 6.92 (s, 1H), 3.72 (mm, 2H), 2.51 (S, 3H), 2.34 (s, 3H), 2.29 (mm, 2H), 1.92 (mm, 4H).

### Synthesis Example 5

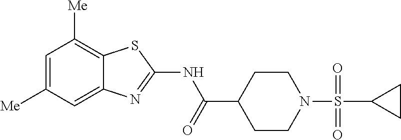 [SMILES: Cc1cc(C)c2sc(NC(=O)C3CCN(S(=O)(=O)C4CC4)CC3)nc2c1]

### VU0943354

1-(Cyclopropylsulfonyl)-N-(5,7-dimethylbenzo[d]thiazol-2-yl)piperidine-4-carboxamide: In a manner similar to that described in Step iii of Example 1, N-(5,7-dimethylbenzo[d]thiazol-2-yl)piperidine-4-carboxamide (40 mg, 0.14 mmol), DIPEA (0.07 mL, 0.42 mmol), cyclopropanesulfonyl chloride (38 mg, 0.27 mmol) and dichloromethane (3.5 mL) afforded the title compound (22 mg, 40%). LC=98% at 254 nm RT=1.01 min, MS=394 (m+1). ¹H NMR (CDCl₃, 400 MHz): δ 7.41 (s, 1H), 6.98 (s, 1H), 3.81 (mm, 2H), 2.80 (mm, 2H), 2.23 (mm, 1H), 1.93 (mm, 4H), 1.16 (dd, J=2.29, J=1.83, 2H), 0.98 (dd, J=1.83 Hz, J=2.74 HZ, 2H).

### Synthesis Example 6

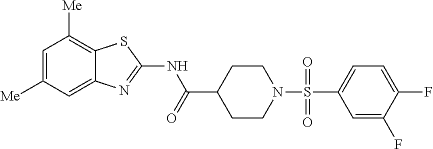 [SMILES: Cc1cc(C)c2sc(NC(=O)C3CCN(S(=O)(=O)c4ccc(F)c(F)c4)CC3)nc2c1]

### VU0943355

1-((3,4-Difluorophenyl)sulfonyl)-N-(5,7-dimethylbenzo[d]thiazol-2-yl)piperidine-4-carboxamide: In a manner similar to that described in Step iii of Example 1, N-(5,7-dimethylbenzo[d]thiazol-2-yl)piperidine-4-carboxamide (40 mg, 0.14 mmol), DIPEA (0.07 mL, 0.42 mmol), 3,4-difluorobenzenesulfonyl chloride (58 mg, 0.27 mmol) and dichloromethane (3.5 mL) afforded the title compound (34 mg, 52%). LC=98% at 254 nm RT=1.14 min, MS=466 (m+1). ¹H NMR (CDCl₃, 400 MHz): δ 7.52 (m, 2H), 7.36 (m, 1H), 7.33 (s, 1H), 6.90 (s, 1H), 3.68 (m, 2H), 2.52 (s, 3H), 2.32 (s, 3H), 2.29 (m, 1H), 2.16 (M, 2H), 1.87 (m, 4H).

### Synthesis Example 7

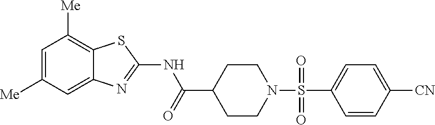 [SMILES: Cc1cc(C)c2sc(NC(=O)C3CCN(S(=O)(=O)c4ccc(C#N)cc4)CC3)nc2c1]

### VU0943356

1-((4-Cyanophenyl)sulfonyl)-N-(5,7-dimethylbenzo[d]thiazol-2-yl)piperidine-4-carboxamide: In a manner similar to that described in Step iii of Example 1, N-(5,7-dimethylbenzo[d]thiazol-2-yl)piperidine-4-carboxamide (40 mg, 0.14 mmol), DIPEA (0.07 mL, 0.42 mmol), 4-cyanobenzenesulfonyl chloride (55 mg, 0.27 mmol) and dichloromethane (3.5 mL) afforded the title compound (24 mg, 38%). LC=99% at 254 nm RT=1.10 min, MS=455 (m+1).

### Synthesis Example 8

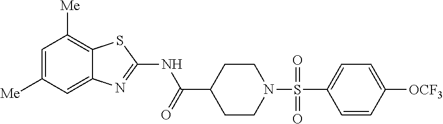 [SMILES: Cc1cc(C)c2sc(NC(=O)C3CCN(S(=O)(=O)c4ccc(OC(F)(F)F)cc4)CC3)nc2c1]

### VU0943357

N-(5,7-Dimethylbenzo[d]thiazol-2-yl)-1-((4-(trifluoromethoxy)phenyl)sulfonyl)piperidine-4-carboxamide: In a manner similar to that described in Step iii of Example 1, N-(5,7-dimethylbenzo[d]thiazol-2-yl)piperidine-4-carboxamide (40 mg, 0.14 mmol), DIPEA (0.07 mL, 0.42 mmol), 4-(trifluoromethoxy)benzenesulfonyl chloride (46 mg, 0.27 mmol) and dichloromethane (3.5 mL) afforded the title compound (26 mg, 36%). LC=96% at 254 nm RT=1.20 min, MS=514 (m+1).

### Synthesis Example 9

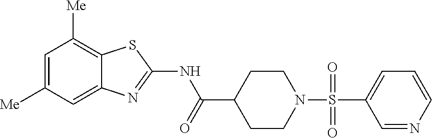 [SMILES: Cc1cc(C)c2sc(NC(=O)C3CCN(S(=O)(=O)c4cccnc4)CC3)nc2c1]

### VU0943360

N-(5,7-Dimethylbenzo[d]thiazol-2-yl)-1-(pyridin-3-ylsulfonyl)piperidine-4-carboxamide: In a manner similar to that described in Step iii of Example 1, N-(5,7-dimethylbenzo[d]thiazol-2-yl)piperidine-4-carboxamide (40 mg, 0.14 mmol), DIPEA (0.07 mL, 0.42 mmol), pyridine-3-sulfonyl chloride (48 mg, 0.27 mmol) and dichloromethane (3.5 mL) afforded the title compound (25 mg, 42%). LC=96% at 254 nm RT=1.01 min, MS=431 (m+1).

### Synthesis Example 10

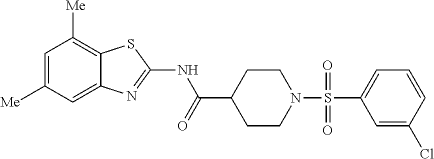 [SMILES: Cc1cc(C)c2sc(NC(=O)C3CCN(S(=O)(=O)c4cccc(Cl)c4)CC3)nc2c1]

### VU0943363

1-((3-Chlorophenyl)sulfonyl)-N-(5,7-dimethylbenzo[d]thiazol-2-yl)piperidine-4-carboxamide: In a manner similar to that described in Step iii of Example 1, N-(5,7-dimethylbenzo[d]thiazol-2-yl)piperidine-4-carboxamide (40 mg, 0.14 mmol), DIPEA (0.07 mL, 0.42 mmol), 3-chlorobenzenesulfonyl chloride (58 mg, 0.27 mmol) and dichloromethane (3.5 mL) afforded the title compound (28 mg, 44%). LC=94% at 254 nm RT=1.15 min, MS=464 (m+1).

### Synthesis Example 11

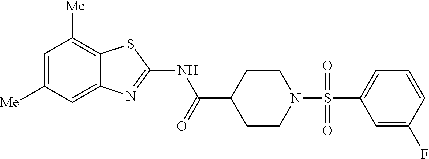 [SMILES: Cc1cc(C)c2sc(NC(=O)C3CCN(S(=O)(=O)c4cccc(F)c4)CC3)nc2c1]

### VU0943358

N-(5,7-Dimethylbenzo[d]thiazol-2-yl)-1-((3-fluorophenyl)sulfonyl)piperidine-4-carboxamide: In a manner similar to that described in Step iii of Example 1, N-(5,7-dimethylbenzo[d]thiazol-2-yl)piperidine-4-carboxamide (40 mg, 0.14 mmol), DIPEA (0.07 mL, 0.42 mmol), 3-fluorobenzenesulfonyl chloride (53 mg, 0.27 mmol) and dichloromethane (3.5 mL) afforded the title compound (33 mg, 53%). LC=95% at 254 nm RT=1.11 min, MS=448 (m+1). ¹H NMR (CDCl₃, 400 MHz): δ 7.53 (m, 2H), 7.38 (m, 2H), 7.32 (s, 1H). 6.87 (s, 1H), 3.70 (m, 2H), 2.51 (s, 3H), 2.28 (s, 3H), 2.09 (m, 2H), 1.84 (m, 5H).

### Synthesis Example 12

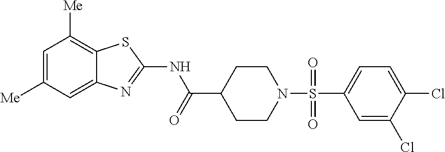 [SMILES: Cc1cc(C)c2sc(NC(=O)C3CCN(S(=O)(=O)c4ccc(Cl)c(Cl)c4)CC3)nc2c1]

### VU0943364

1-((3,4-Dichlorophenyl)sulfonyl)-N-(5,7-dimethylbenzo[d]thiazol-2-yl)piperidine-4-carboxamide: In a manner similar to that described in Step iii of Example 1, N-(5,7-dimethylbenzo[d]thiazol-2-yl)piperidine-4-carboxamide (40 mg, 0.14 mmol), DIPEA (0.07 mL, 0.42 mmol), 3,4-dichlorobenzenesulfonyl chloride (67 mg, 0.27 mmol) and dichloromethane (3.5 mL) afforded the title compound (16 mg, 23%). LC=97% at 254 nm RT=1.20 min, MS=498 (m+1). ¹H NMR (CDCl₃, 400 MHz): δ 7.82 (d, J=1.67 Hz, 1H), 7.62 (d, J=7.89 Hz, 1H), 7.53 (dd. J=1.67 Hz, J=2.07 Hz, 1H), 7.33 (s, 1H), 6.90 (s, 1H), 3.71 (m, 2H), 2.51 (s, 3H), 2.34 (m, 1H), 2.32 (s, 3H), 2.21 (m, 2H), 1.90 (m, 4H).

### Synthesis Example 13

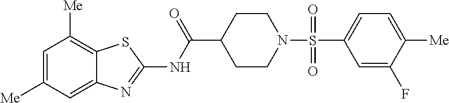 [SMILES: Cc1cc(C)c2sc(NC(=O)C3CCN(S(=O)(=O)c4ccc(C)c(F)c4)CC3)nc2c1]

### VU0944252

N-(5,7-Dimethylbenzo[d]thiazol-2-yl)-1-((3-fluoro-4-methylphenyl)sulfonyl)piperidine-4-carboxamide: In a manner similar to that described in Step iii of Example 1, N-(5,7-dimethylbenzo[d]thiazol-2-yl)piperidine-4-carboxamide (33 mg, 0.11 mmol), DIPEA (0.06 mL, 0.34 mmol), 3-fluoro-4-methylbenzenesulfonyl chloride (47 mg, 0.23 mmol) and dichloromethane (3.5 mL) afforded the title compound (39 mg, 74%). LC=95% at 254 nm RT=1.15 min, MS=462 (m+1). ¹H NMR (CDCl₃, 400 MHz): δ 7.38 (m, 2H), 7.36 (m, 1H), 7.26 (s, 1H), 6.86 (s, 1H), 3.67 (m, 2H), 2.51 (s, 3H), 2.41 (s, 3H), 2.27, (s, 3H), 2.23 (m, 1H), 2.07 (m, 2H), 1.83 (m, 4H).

### Synthesis Example 14

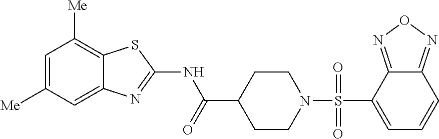 [SMILES: Cc1cc(C)c2sc(NC(=O)C3CCN(S(=O)(=O)c4cccc5nonc45)CC3)nc2c1]

### VU0944253

1-(Benzo[c][1,2,5]oxadiazol-4-ylsulfonyl)-N-(5,7-dimethylbenzo[d]thiazol-2-yl)piperidine-4-carboxamide: In a manner similar to that described in Step iii of Example 1, N-(5,7-dimethylbenzo[d]thiazol-2-yl)piperidine-4-carboxamide (33 mg, 0.11 mmol), DIPEA (0.06 mL, 0.34 mmol), benzo[c][1,2,5]oxadiazole-4-sulfonyl chloride (49 mg, 0.23 mmol) and dichloromethane (3.5 mL) afforded the title compound (17 mg, 32%). LC=94% at 254 nm RT=1.09 min, MS=472 (m+1). ¹H NMR (CDCl₃, 400 MHz): δ 8.12 (d, J=8.3 Hz, 1H), 7.99 (d, J=7.1, 1H), 7.56 (t, J=6.5 Hz, J=8.28 Hz), 1H), 7.32 (s, 1H), 6.81 (s, 1H), 3.96 (m, 2H), 2.57 (m, 2H), 2.49 (s, 3H), 2.39 (m, 1H), 2.27 (s, 3H), 1.86 (m, 4H).

### Synthesis Example 15

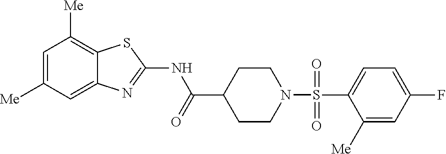 [SMILES: Cc1cc(C)c2sc(NC(=O)C3CCN(S(=O)(=O)c4ccc(F)cc4C)CC3)nc2c1]

### VU0944247

N-(5,7-Dimethylbenzo[d]thiazol-2-yl)-1-((4-fluoro-2-methylphenyl)sulfonyl)piperidine-4-carboxamide: In a manner similar to that described in Step iii of Example 1, N-(5,7-dimethylbenzo[d]thiazol-2-yl)piperidine-4-carboxamide (33 mg, 0.11 mmol), DIPEA (0.06 mL, 0.34 mmol), 4-fluoro-2-methylbenzenesulfonyl chloride (47 mg, 0.23 mmol) and dichloromethane (3.5 ml) afforded the title compound (22 mg, 42%). LC=94% at 254 nm RT=1.15 min, MS=463 (m+1). ¹H NMR (CDCl₃, 400 MHz): δ 7.88 (t, J=8.5 Hz, J=7.11 Hz, 1H), 7.37 (s, 1H), 7.02 (m, 2H), 6.95 (s, 1H), 3.64 (m, 2H), 257 (s, 3H), 2.52 (s, 3H), 2.44 (m, 3H), 2.39 (s, 3H), 1.82 (m, 4H).

### Synthesis Example 16

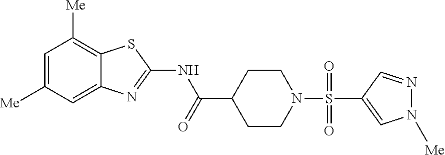 [SMILES: Cc1cc(C)c2sc(NC(=O)C3CCN(S(=O)(=O)c4cnn(C)c4)CC3)nc2c1]

### VU00944254

N-(5,7-Dimethylbenzo[d]thiazol-2-yl)-1-((1-methyl-1H-pyrazol-4-yl)sulfonyl)piperidine-4-carboxamide: In a manner similar to that described in Step iii of Example 1, N-(5,7-dimethylbenzo[d]thiazol-2-yl)piperidine-4-carboxamide (33 mg, 0.11 mmol), DIPEA (0.06 mL, 0.34 mmol), 1-methyl-1H-pyrazole-4-sulfonyl chloride (41 mg, 0.23 mmol) and dichloromethane (3.5 ml) afforded the title compound (9 mg, 17%). LC=90% at 254 nm RT=0.99 min, MS=434 (m+1). ¹H NMR (CDCl₃), 400 MHz): ð 7.71 (s, 1H), 7.66 (s, 1H), 7.37 (s, 1H), 6.9 (s, 1H), 3.98 (s, 3H), 3.66 (m, 2H), 2.53 (s, 3H), 2.37 (s, 3H), 2.33 (m, 1H), 2.26 (s, 2H), 1.93 (m, 4H).

### Synthesis Example 17

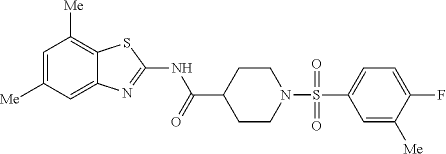 [SMILES: Cc1cc(C)c2sc(NC(=O)C3CCN(S(=O)(=O)c4ccc(F)c(C)c4)CC3)nc2c1]

### VU0944255

N-(5,7-Dimethylbenzo[d]thiazol-2-yl)-1-((4-fluoro-3-methylphenyl)sulfonyl)piperidine-4-carboxamide: In a manner similar to that described in Step iii of Example 1, N-(5,7-dimethylbenzo[d]thiazol-2-yl)piperidine-4-carboxamide (33 mg, 0.11 mmol), DIPEA (0.06 mL, 0.34 mmol), 4-fluoro-3-methylbenzenesulfonyl chloride (47 mg, 0.23 mmol) and dichloromethane (3.5 mL) afforded the title compound (30 mg, 57%). LC=96% at 254 nm RT=1.14 min, MS=463 (m+1). ¹H NMR (CDCl₃), 400 MHz): δ 7.57 (d, J=8.29 Hz, 1H), 7.53 (m, 1H), 7.32 (S. 1H), 7.15 (t, J=9.46, J=8.29, 1H), 6.88 (s, 1H), 3.69 (m, 2H), 2.51 (s, 3H), 2.36 (s, 3H), 2.33 (m, 1H), 2.29 (s, 3H), 2.15 (m, 2H), 1.86 (m, 4H).

### Synthesis Example 18

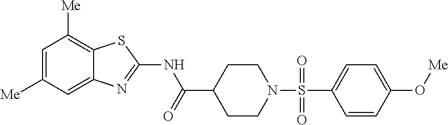 [SMILES: COc1ccc(S(=O)(=O)N2CCC(C(=O)Nc3nc4cc(C)cc(C)c4s3)CC2)cc1]

### VU0944256

N-(5,7-Dimethylbenzo[d]thiazol-2-yl)-1-((4-methoxyphenyl)sulfonyl)piperidine-4-carboxamide: In a manner similar to that described in Step iii of Example 1, N-(5,7-dimethylbenzo[d]thiazol-2-yl)piperidine-4-carboxamide (33 mg, 0.11 mmol), DIPEA (0.06 mL, 0.34 mmol), 4-methoxybenzenesulfonyl chloride (46 mg, 0.23 mmol) and dichloromethane (3.5 mL) afforded the title compound (15 mg, 28%). LC=99% at 254 nm RT=1.10 min, MS=460 (m+1).

### Synthesis Example 19

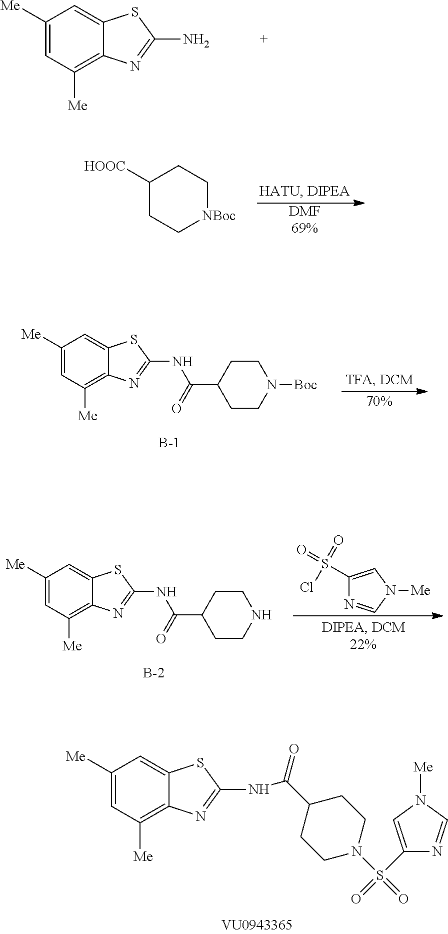

i. Preparation of tert-butyl 4-((4,6-dimethylbenzo[d]thiazol-2-yl) carbamoyl)piperidine-1-carboxylate (B-1): To a solution of 1-(tert-butoxycarbonyl)piperidine-4-carboxylic acid (2.81 g, 12.29 mmol mmol) in DMF (30 mL) at 0° C. was added DIPEA (4.33 mL, 24.58 mmol) and HATU (5.6 g, 14.75 mmol). After stirring for 10 min at 0° C., 4,6-dimethylbenzo[d]thiazol-2-amine (2.0 g, 11.17 mmol) was added to reaction mixture. The reaction mixture was stirred for 48 h at room temperature, poured onto ice and then extracted with EtOAc (3×40 mL), The combined extracts were washed with water (20 mL), 1 N HCl (20 mL), 1 N NaOH (20 mL), and brine (20 mL). The combined organic layer was dried over MgSO₄, concentrated in vacuo, and purified on a Teledyne ISCO Combi-Flash system via normal phase chromatography on silica gel (EtOAc/hexanes) to afford B-1 as a white solid (3.01 g, 69%). LCMS calculated for C₂₀H₂₇N₃O₃S [M+H]⁺: 389.5 measured 390.5. ¹H NMR (DMSO-d₆), 400 MHz): δ 7.57 (s, 1H), 7.07 (s, 1H), 3.99 (m, 2H), 2.73 (m, 2H), 2.56 (s, 3H), 2.36 (s, 3H), 1.83 (m, 1H), 1.51 (m, 4H) 1.40 (s, 9H).

ii. Preparation of N-(4,6-dimethylbenzo[d]thiazol-2-yl)piperidine-4-carboxamide (B-2). To a solution of carbamate B-1 (3.01 g, 7.71 mmol) in dichloromethane (30 mL) was added TFA (30 mL, 9.41 mmol) at room temperature. The reaction mixture was heated at ambient temperature for 1 h and then concentrated in vacuo. The residue was dissolved in dichloromethane (50 mL) and treated with 1 N NaOH until the aqueous layer was basic. The solution was filtered, dried over MgSO₄, filtered, and concentrated in vacuo to afford the title compound (1.57 g, 70%.

iii. Preparation of the title compound, N-(4,6-dimethylbenzo[d]thiazol-2-yl)-1-((1-methyl-1H-imidazol-4-yl)sulfonyl)piperidine-4-carboxamide (VU0943365): To a solution of N-(4,6-dimethylbenzo[d]thiazol-2-yl)piperidine-4-carboxamide (50 mg, 0.12 mmol) in THF (3 mL) was added DIPEA (0.11 mL, 0.62 mmol) and 1-methyl-1H-imidazole-4-sulfonyl chloride (33 mg, 0.18 mmol) at 0° C. The reaction mixture was stirred at room temperature for 48 h, dissolved in DCM (10 mL), extracted with 1 N NaOH, dried over MgSO₄, concentrated in vacuo, and purified on a Teledyne ISCO Combi-Flash system via normal phase chromatography on silica gel (DCM/MeOH) to afford the title compound as a white solid (12 mg, 22%). LC=99% at 254 nm RT=0.98 min, MS=434 (m+1). ¹H NMR (CDCl₃, 400 MHz): δ 7.53 (d, J=1.23 Hz, 1H), 7.45 (d, J=1.18 Hz, 1H), 7.43 (s, 1H), 7.07 (s, 1H), 3.94 (m, 1H), 9.91 (m, 1H), 3.94 (mm, 2H), 3.77 (s, 3H), 1.96 (mm, 2H), 2.58 (s, 3H), 2.42 (s, 3H), 2.00 M, 4H).

### Synthesis Example 20

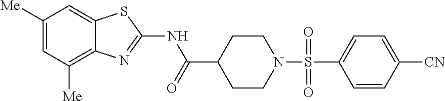 [SMILES: Cc1cc(C)c2nc(NC(=O)C3CCN(S(=O)(=O)c4ccc(C#N)cc4)CC3)sc2c1]

### VU0944248

1-((4-Cyanophenyl)sulfonyl)-N-(4,6-dimethylbenzo[d]thiazol-2-yl)piperidine-4-carboxamide: In a manner similar to that described in Example 19, N-(4,6-dimethylbenzo[d]thiazol-2-yl)piperidine-4-carboxamide (B-2) (75 mg, 0.19 mmol), DIPEA (0.08 mL, 0.46 mmol), 4-cyanobenzenesulfonyl chloride (56 mg, 0.28 mmol) and dichloromethane (3.5 mL) afforded the title compound (30 mg, 35%). LC=90% at 254 nm RT=1.09 min, MS=455 (m+1). ¹H NMR (CDCl₃), 400 MHz): δ 7.88 (m, 4H), 7.43 (s, 1H), 7.07 (s, 1H), 3.79 (m, 2H), 2.63 (m, 2H), 2.56 (s, 3H), 2.43 (s, 3H), 2.37 (m, 1H) 2.05 (m, 4H).

### Synthesis Example 21

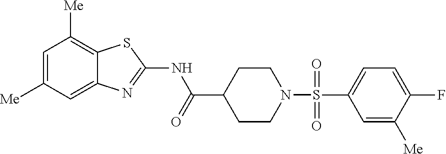 [SMILES: Cc1cc(C)c2sc(NC(=O)C3CCN(S(=O)(=O)c4ccc(F)c(C)c4)CC3)nc2c1]

### VU0944249

N-(5,7-Dimethylbenzo[d]thiazol-2-yl)-1-((4-fluoro-3-methylphenyl)sulfonyl)piperidine-4-carboxamide: In a manner similar to that described in Example 19, N-(5,7-dimethylbenzo[d]thiazol-2-yl)piperidine-4-carboxamide (B-2) (75 mg, 0.19 mmol), DIPEA (0.08 mL, 0.46 mmol), 4-fluoro-3-methylbenzenesulfonyl chloride (58 mg, 0.28 mmol) and dichloromethane (3.5 mL) afforded the title compound (70 mg, 82%). LC=98% at 254 nm RT=1.16 min, MS=461 (m+1).

### Synthesis Example 22

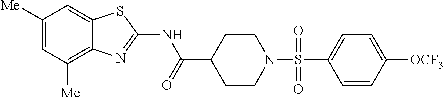 [SMILES: Cc1cc(C)c2nc(NC(=O)C3CCN(S(=O)(=O)c4ccc(OC(F)(F)F)cc4)CC3)sc2c1]

### VU0944250

N-(4,6-Dimethylbenzo[d]thiazol-2-yl)-1-((4-(trifluoromethoxy)phenyl)sulfonyl)piperidine-4-carboxamide: In a manner r similar to that described in Example 19, N-(4,6-dimethylbenzo[d]thiazol-2-yl)piperidine-4-carboxamide (B-2) (75 mg, 0.19 mmol), DIPEA (0.08 mL, 0.46 mmol), 4-(trifluoromethoxy)benzenesulfonyl chloride (58 mg, 0.28 mmol) and dichloromethane (3.5 mL) afforded the title compound (50 mg, 56%). LC=99% at 254 nm RT=1.18 min, MS=514 (m+1). ¹H NMR (CDCl₃), 400 MHz): δ 7.83 (d, J=8.29, 2H), 7.43 (s, 1H), 7.37 (d, J=8.28 Hz, 2H), 7.05 (s, 1H), 3.79 (m, 2H), 2.56 (s, 3H), 2.51 (m, 2H), 2.41 (s, 3H), 2.34 (m, 1H), 2.00 (m, 4H).

### Synthesis Example 23

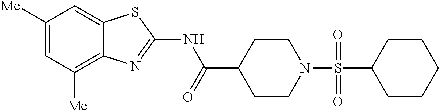 [SMILES: Cc1cc(C)c2nc(NC(=O)C3CCN(S(=O)(=O)C4CCCCC4)CC3)sc2c1]

### VU0944243

1-(Cyclohexylsulfonyl)-N-(4,6-dimethylbenzo[d]thiazol-2-yl)piperidine-4-carboxamide: In a manner similar to that described in Example 19, N-(4,6-dimethylbenzo[d]thiazol-2-yl)piperidine-4-carboxamide (B-2) (75 mg, 0.19 mmol), DIPEA (0.08 mL, 0.46 mmol), cyclohexanesulfonyl chloride (58 mg, 0.28 mmol) and dichloromethane (3.5 mL) afforded the title compound (20 mg, 24%). LC=91% at 254 nm RT=1.13 min, MS=436 (m+1).

### Synthesis Example 24

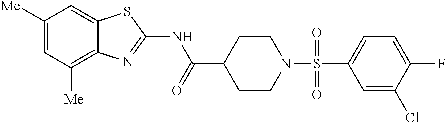 [SMILES: Cc1cc(C)c2nc(NC(=O)C3CCN(S(=O)(=O)c4ccc(F)c(Cl)c4)CC3)sc2c1]

### VU0944295

1-((3-Chloro-4-fluorophenyl)sulfonyl)-N-(4,6-dimethylbenzo[d]thiazol-2-yl)piperidine-4-carboxamide: In a manner similar to that described in Example 19, N-(4,6-dimethylbenzo[d]thiazol-2-yl)piperidine-4-carboxamide (B-2) (50 mg, 0.17 mmol), DIPEA (0.09 mL, 0.51 mmol), 3-chloro-4-fluorobenzenesulfonyl chloride (77 mg, 0.34 mmol) and dichloromethane (3.5 mL) afforded the title compound (25 mg, 30%). LC=91% at 254 nm RT=1.18 min, MS=482 (m+1). ¹H NMR (CDCl₃), 400 MHz): δ 7.85 (dd, J=1.78 Hz, J=3.18 Hz, 1H), 7.67 (m, 1H), 7.43 (s, 1H), 7.31 (t, J=8.29 Hz, J=8.58 Hz, 1H), 7.06 (s, 1H), 3.77 (m, 2H), 2.56 (s, 3H), 2.52 (m, 2H), 2.41 (s, 3H), 2.34 (m, 1H), 2.00 (m, 4H).

### Synthesis Example 25

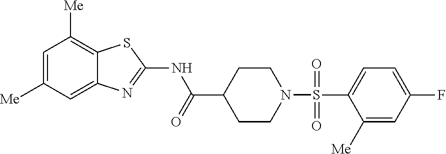 [SMILES: Cc1cc(C)c2sc(NC(=O)C3CCN(S(=O)(=O)c4ccc(F)cc4C)CC3)nc2c1]

### VU0944247

N-(5,7-Dimethylbenzo[d]thiazol-2-yl)-1-((4-fluoro-2-methylphenyl)sulfonyl)piperidine-4. carboxamide: In a manner similar to that described in Example 19, N-(5,7-dimethylbenzo[d]thiazol-2-yl)piperidine-4-carboxamide (33 mg, 0.11 mmol), DIPEA (0.06 mL, 0.34 mmol), 4-methoxybenzenesulfonyl chloride (47 mg, 0.23 mmol) and dichloromethane (3.5 mL) afforded the title compound (26 mg, 51%). LC=94% at 254 nm RT=1.15 min, MS=462 (m+1).

### Synthesis Example 26

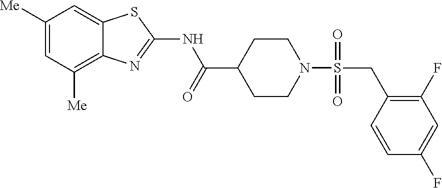 [SMILES: Cc1cc(C)c2nc(NC(=O)C3CCN(S(=O)(=O)Cc4ccc(F)cc4F)CC3)sc2c1]

### VU0944257

1-((2,4-Difluorobenzyl)sulfonyl)-N-(4,6-dimethylbenzo[d]thiazol-2-yl)piperidine-4-carboxamide: In a manner similar to that described in Example 19, N-(4,6-dimethylbenzo[d]thiazol-2-yl)piperidine-4-carboxamide (B-2) (40 mg, 0.14 mmol), DIPEA (0.07 mL, 0.41 mmol), (2,4-difluorophenyl) methanesulfonyl chloride (62 mg, 0.28 mmol) and dichloromethane (3.0 mL) afforded the title compound (21 mg, 32%). LC=98% at 254 nm RT=1.12 min, MS=480 (m+1).

### Synthesis Example 27

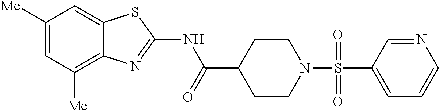 [SMILES: Cc1cc(C)c2nc(NC(=O)C3CCN(S(=O)(=O)c4cccnc4)CC3)sc2c1]

### VU0944258

N-(4,6-Dimethylbenzo[d]thiazol-2-yl)-1-(pyridin-3-ylsulfonyl)piperidine-4-carboxamide: In a manner similar to that described in Example 19, N-(4,6-dimethylbenzo[d]thiazol-2-yl)piperidine-4-carboxamide (B-2) (40 mg, 0.14 mmol), DIPEA (0.07 mL, 0.41 mmol), pyridine-3-sulfonyl chloride (48 mg, 0.28 mmol) and dichloromethane (3.0 mL) afforded the title compound (54 mg, 84%). LC=98% at 254 nm RT=1.03 min, MS=465 (m+1).

### Synthesis Example 28

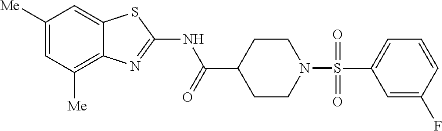 [SMILES: Cc1cc(C)c2nc(NC(=O)C3CCN(S(=O)(=O)c4cccc(F)c4)CC3)sc2c1]

### VU0944244

N-(4,6-Dimethylbenzo[d]thiazol-2-yl)-1-((3-fluorophenyl)sulfonyl)piperidine-4-carboxamide: In a manner similar to that described in Example 19, N-(4,6-dimethylbenzo[d]thiazol-2-yl)piperidine-4-carboxamide (B-2) (50 mg, 0.17 mmol), DIPEA (0.05 mL, 0.89 mmol), 3-fluorobenzenesulfonyl chloride (66 mg, 0.34 mmol) and dichloromethane (3.0 mL) afforded the title compound (27 mg, 35%). LC=98% at 254 nm RT=1.12 min, MS=448 (m+1).

### Synthesis Example 29

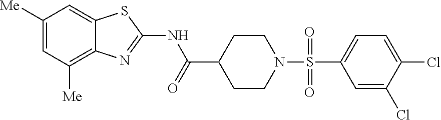 [SMILES: Cc1cc(C)c2nc(NC(=O)C3CCN(S(=O)(=O)c4ccc(Cl)c(Cl)c4)CC3)sc2c1]

### VU0944245

1-((3,4-Dichlorophenyl)sulfonyl)-N-(4,6-dimethylbenzo[d]thiazol-2-yl)piperidine-4-carboxamide: In a manner similar to that described in Example 19, N-(4,6-dimethylbenzo[d]thiazol-2-yl)piperidine-4-carboxamide (B-2) (50 mg, 0.17 mmol), DIPEA (0.05 mL, 0.89 mmol), 3,4-dichlorobenzenesulfonyl chloride (83 mg, 0.34 mmol) and dichloromethane (3.0 mL) afforded the title compound (27 mg, 32%). LC=98% at 254 nm RT=1.20 min, MS=498 (m+1).

### Synthesis Example 30

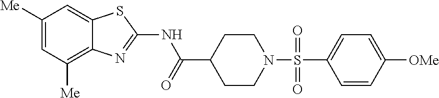 [SMILES: COc1ccc(S(=O)(=O)N2CCC(C(=O)Nc3nc4c(C)cc(C)cc4s3)CC2)cc1]

### VU0944246

N-(4,6-Dimethylbenzo[d]thiazol-2-yl)-1-((4-methoxyphenyl)sulfonyl)piperidine-4-carboxamide: In a manner similar to that described in Example 19, N-(4,6-dimethylbenzo[d]thiazol-2-yl)piperidine-4-carboxamide (B-2) (50 mg, 0.17 mmol), DIPEA (0.09 mL, 0.89 mmol), 4-methoxybenzenesulfonyl chloride (71 mg, 0.34 mmol) and dichloromethane (3.0 mL) afforded the title compound (15 mg, 19%). LC=98% at 254 nm RT=1.20 min, MS=464 (m+1).

### Synthesis Example 31

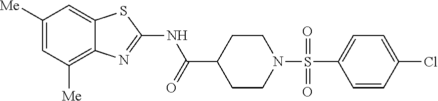 [SMILES: Cc1cc(C)c2nc(NC(=O)C3CCN(S(=O)(=O)c4ccc(Cl)cc4)CC3)sc2c1]

### VU0944259

1-((4-Chlorophenyl)sulfonyl)-N-(4,6-dimethylbenzo[d]thiazol-2-yl)piperidine-4-carboxamide: In a manner similar to that described in Example 19, N-(4,6-dimethylbenzo[d]thiazol-2-yl)piperidine-4-carboxamide (B-2) (50 mg, 0.17 mmol), DIPEA (0.09 mL, 0.89 mmol), 4-chlorobenzenesulfonyl chloride (71 mg, 0.34 mmol) and dichloromethane (3.0 mL) afforded the title compound (13 mg, 17%). LC=98% at 254 nm RT=1.20 min, MS=464 (m+1). ¹H NMR (CDCl₃), 400 MHz): δ 7.71 (d, J=8.24 Hz, 2H), 7.52 (d, J=9.46, 2H), 7.43 (s, 1H), 7.05 (s, 1H), 3.78 (m, 2H), 2.56 (s, 3H), 2.47 (m, 2H), 2.41 (s, 3H), 2.31 (m, 1H), 1.99 (m, 4H).

### Synthesis Example 32

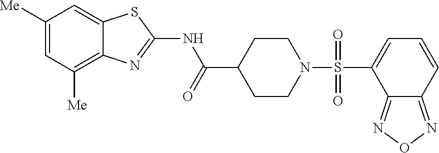 [SMILES: Cc1cc(C)c2nc(NC(=O)C3CCN(S(=O)(=O)c4cccc5nonc45)CC3)sc2c1]

### VU0944292

1-(Benzo[c][1,2,5]oxadiazol-4-ylsulfonyl)-N-(4,6-dimethylbenzo[d]thiazol-2-yl)piperidine-4-carboxamide: In a manner similar to that described in Example 19, N-(4,6-dimethylbenzo[d]thiazol-2-yl)piperidine-4-carboxamide (B-2) (50 mg, 0.17 mmol), DIPEA (0.09 mL, 0.89 mmol), benzo[c][1,2,5]oxadiazole-4-sulfonyl chloride (74 mg, 0.34 mmol) and dichloromethane (3.0 mL) afforded the title compound (32 mg, 40%). LC=98% at 254 nm RT=1.09 min, MS=472 (m+1). ¹H NMR (CDCl₃), 400 MHz): δ 8.10 (d, J=9.47 Hz, 1H), 8.04 (d, J=5.92 Hz, 1H), 7.56 (m, 1H), 7.43 (s, 1H), 7.06 (s, 1H), 4.06 (m, 2H), 2.95 (m, 2H), 2.56 (s, 3H), 2.44 (m, 1H), 2.41 (s, 3H), 2.00 (m, 4H).

### Synthesis Example 33

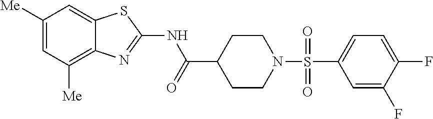 [SMILES: Cc1cc(C)c2nc(NC(=O)C3CCN(S(=O)(=O)c4ccc(F)c(F)c4)CC3)sc2c1]

### VU0944294

1-((3,4-Difluorophenyl)sulfonyl)-N-(4,6-dimethylbenzo[d]thiazol-2-yl)piperidine-4-carboxamide: In a manner similar to that described in Example 19, N-(4,6-dimethylbenzo[d]thiazol-2-yl)piperidine-4-carboxamide (B-2) (50 mg, 0.17 mmol), DIPEA (0.09 mL, 0.89 mmol), 3,4-difluorobenzenesulfonyl chloride (72 mg, 0.34 mmol) and dichloromethane (3.0 mL) afforded the title compound (31 mg, 31%). LC=98% at 254 nm RT=1.13 min, MS=466 (m+1).

### Synthesis Example 34

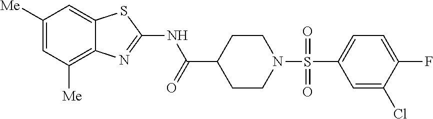 [SMILES: Cc1cc(C)c2nc(NC(=O)C3CCN(S(=O)(=O)c4ccc(F)c(Cl)c4)CC3)sc2c1]

### VU0944295

1-((3-Chloro-4-fluorophenyl)sulfonyl)-N-(4,6-dimethylbenzo[d]thiazol-2-yl)piperidine-4-carboxamide: In a manner similar to that described in Example 19, N-(4,6-dimethylbenzo[d]thiazol-2-yl)piperidine-4-carboxamide (B-2) (50 mg, 0.17 mmol), DIPEA (0.09 mL, 0.89 mmol), 3-chloro-4-fluorobenzenesulfonyl chloride (77 mg, 0.34 mmol) and dichloromethane (3.0 mL) afforded the title compound (25 mg, 30%). LC=92% at 254 nm RT=1.18 min, MS=482 (m+1). ¹H NMR (CDCl₃), 400 MHz): δ 7.85 (dd, J=2.37 Hz, J=1.77 HZ, 1H), 7.66 (m, 1H), 7.43 (s, 1H), 7.31 (t, J=7.68 Hz, J=9.47 Hz, 1H), 7.06 (s, 1H), 3.77 (m, 2H), 2.56 (s, 3H), 2.51, m, 2H), 2.41 (s, 3H), 2.34 (m, 1H), 2.00 (m, 4H).

### Synthesis Example 35

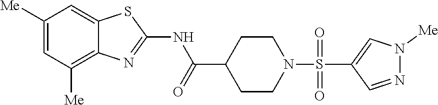 [SMILES: Cc1cc(C)c2nc(NC(=O)C3CCN(S(=O)(=O)c4cnn(C)c4)CC3)sc2c1]

### VU0944293

N-(4,6-Dimethylbenzo[d]thiazol-2-yl)-1-((1-methyl-1H-pyrazol-4-yl)sulfonyl)piperidine-4-carboxamide: In a manner similar to that described in Example 19, N-(4,6-dimethylbenzo[d]thiazol-2-yl)piperidine-4-carboxamide (B-2) (50 mg, 0.17 mmol), DIPEA (0.09 mL, 0.89 mmol), 1-methyl-1H-pyrazole-4-sulfonyl chloride (77 mg, 0.34 mmol) and dichloromethane (3.0 mL) afforded the title compound (21 mg, 30%). LC=98% at 254 nm RT=0.95 min, MS=434 (m+1).

### Synthesis Example 36

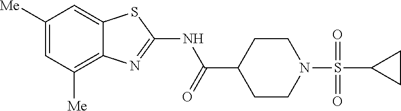 [SMILES: Cc1cc(C)c2nc(NC(=O)C3CCN(S(=O)(=O)C4CC4)CC3)sc2c1]

### VU0944296

1-(Cyclopropylsulfonyl)-N-(4,6-dimethylbenzo[d]thiazol-2-yl)piperidine-4-carboxamide: In a manner similar to that described in Example 19, N-(4,6-dimethylbenzo[d]thiazol-2-yl)piperidine-4-carboxamide (B-2) (50 mg, 0.17 mmol), DIPEA (0.09 mL, 0.89 mmol), cyclopropanesulfonyl chloride (47 mg, 0.34 mmol) and dichloromethane (3.0 mL) afforded the title compound (24 mg, 36%). LC=98% at 254 nm RT=1.02 min, MS=394 (m+1). ¹H NMR (CDCl₃), 400 MHz): δ 9.05 (s, 1H), 7.45 (s, 1H), 7.10 (s, 1H), 3.85 (m, 2H), 2.95 (t, J=10.65 Hz, J=11.84 Hz, 1H), 2.59 (s, 3H), 2.50 (m, 1H), 2.43 (s, 3H), 2.22 (m, 2H), 2.02 (m, 4H), 1.19 (m, 2H), 1.01 (d, J=7.1 Hz, 2H).

### Synthesis Example 37

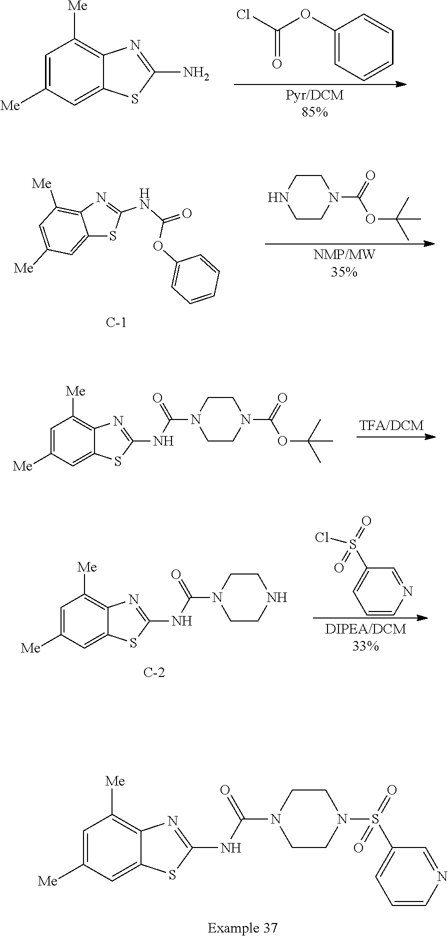

i. Preparation of phenyl (4,6-dimethylbenzo[d]thiazol-2-yl) carbamate (C-1): To a solution of 5,7-dimethylbenzo[d]thiazol-2-amine (0.5 g, 2.8 mmol) in DCM (20 mL) was added pyridine (0.24 g, 3.08 mmol) followed by phenyl carbonochloridate (0.40 mL, 3.08 mmol). The reaction was allowed to stir at ambient temperature for 48 hours. The solution was extracted with 1 N HCl (5 mL), brine, and dried over MgSO₄. The filtered solution was concentrated in vacuo and purified on silica gel using ethyl acetate/hexanes as a mobile phase to afford the title compound (0.71 g, 85%). ¹H NMR (DMSO-d₆), 400 MHz): δ 7.56 (s, 1H), 7.46 (t, J=10.65 Hz, J=6.65 Hz, 2H), 7.31 (m, 1H), 7.29 (m, 2H), 2.54 (s, 3H), 2.36 (s, 3H).

ii. Preparation of N-(4,6-dimethylbenzo[d]thiazol-2-yl) piperazine-1-carboxamide (C-2): A solution of phenyl (5,7-dimethylbenzo[d]thiazol-2-yl) carbamate (0.2 g, 0.67 mmol), tert-butyl piperazine-1-carboxylate (0.18 g, 0.80 mmol) in NMP (2 mL) was heated for 10 min at 1000° C. on a microwave machine. The reaction was poured onto water (10 mL) and extracted with ethyl acetate (3× 10 mL). The organics were extracted with brine (10 mL) dried over MgSO₄, filtered, and concentrated in vacuo. The crude was purified on silica gel using ethyl acetate/hexanes to afford tert-butyl 4-((4,6-dimethylbenzo[d]thiazol-2-yl) carbamoyl) piperazine-1-carboxylate (0.09 g, 35), MS=391 (m+1). The residue was dissolved in DCM (3 mL), cooled on ice, and treated with TFA (2 mL). The mixture was stirred for 2 hours then the reaction was concentrated in vacuo to afford the title compound (0.16 g, >100%). ¹H NMR (DMSO-d₆), 400 MHz): δ 7.45 (s, 1H), 7.01 (s, 1H), 3.77 (m, 4H), 3.16 (m, 4H), 2.48 (s, 3H), 2.33 (s, 3H).

iii. Preparation of the title compound, N-(4,6-dimethylbenzo[d]thiazol-2-yl)-4-(pyridin-3-ylsulfonyl) piperazine-1-carboxamide: In a manner similar to that described in Example 19, N-(4,6-dimethylbenzo[d]thiazol-2-yl) piperazine-1-carboxamide (74 mg, 0.18 mmol), DIPEA (0.09 mL, 0.54 mmol), pyridine-3-sulfonyl chloride (65 mg, 0.36 mmol) and dichloromethane (3.0 mL) afforded the title compound (26 mg, 33%). LC=99% at 254 nm RT=0.97 min, MS=432 (m+1). ¹H NMR (DMSO-d₆), 400 MHz): δ 8.93 (d, J=1.78 Hz, 1H), 8.89 (dd, J=1.19 Hz, J=1.18 Hz, 1H), 8.18 (d, J=8.29 Hz, 1H), 7.70) m, 1H), 7.43 (s, 1H), 6.98 (s, 1H) 3.65 (s, 4H), 3.02 (s, 4H), 2.45 (s, 3H), 2.31 (s, 3H).

All of the compounds shown below may be prepared according to the methods described above, with appropriate starting materials.

### 4,6-Dimethyl Substituted Compounds:

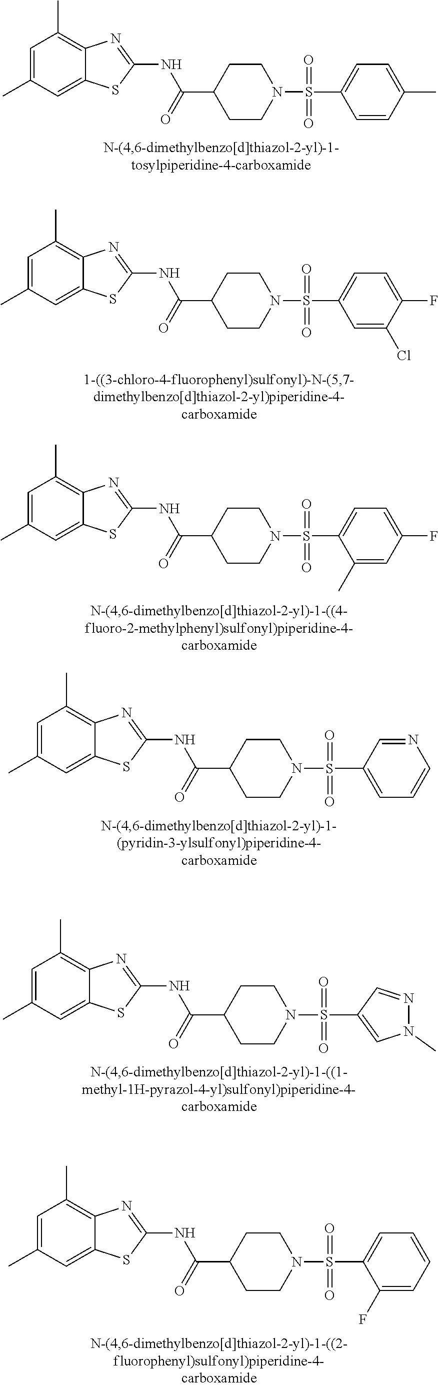

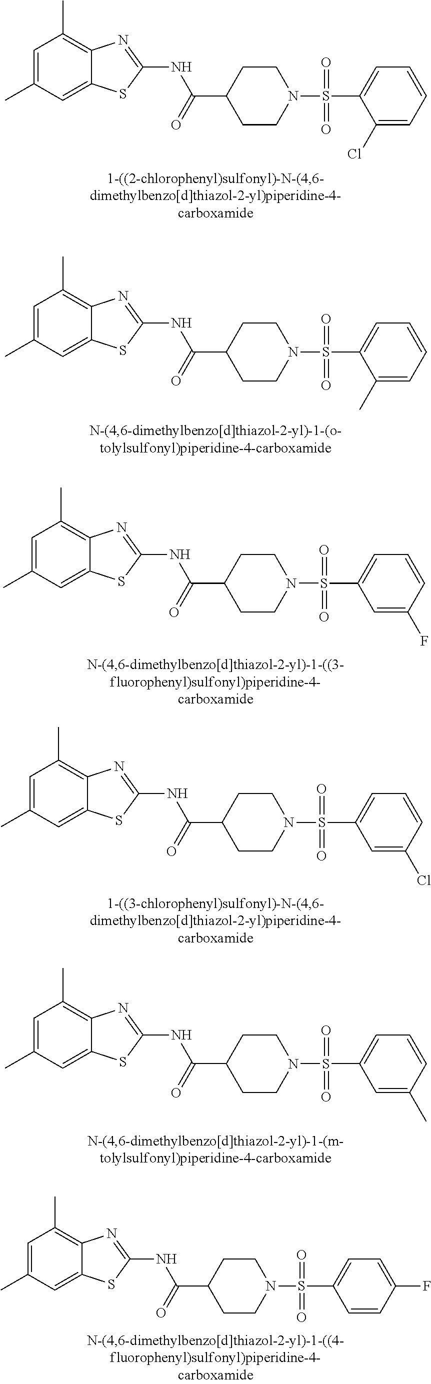

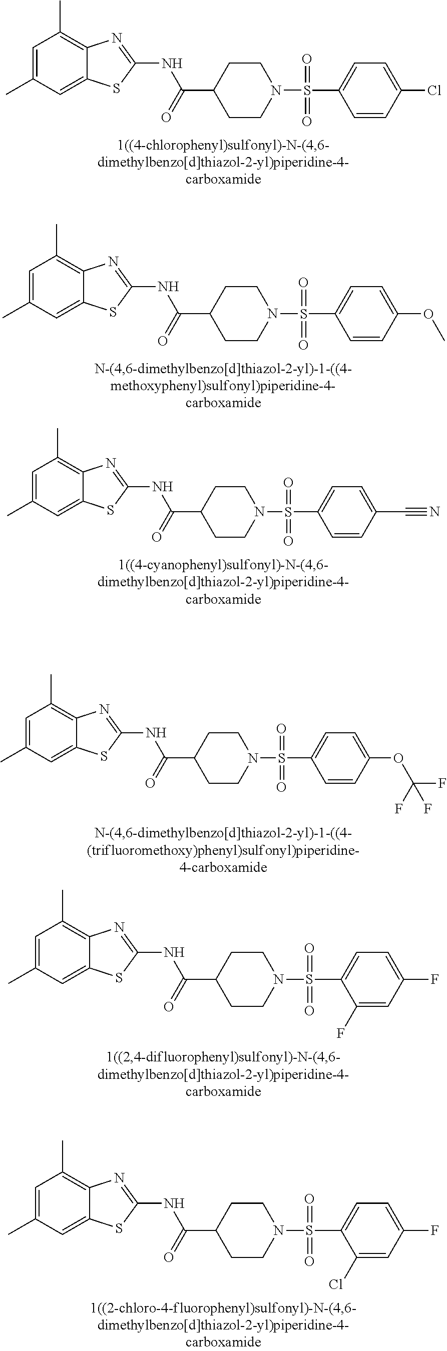

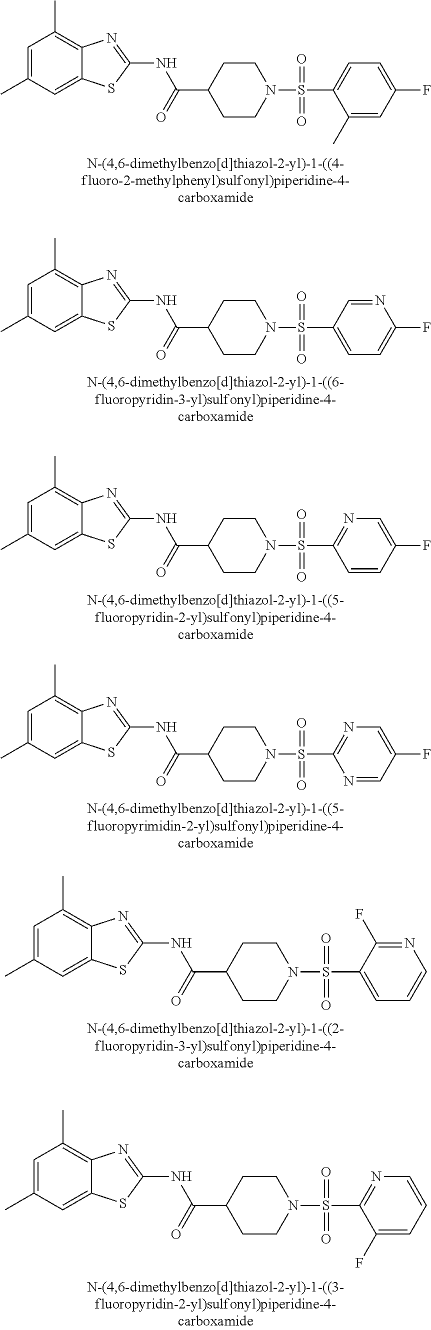 [SMILES: Cc1cc(C)c2nc(NC(=O)C3CCN(S(=O)(=O)c4ccc(F)cc4C)CC3)sc2c1 ; Cc1cc(C)c2nc(NC(=O)C3CCN(S(=O)(=O)c4ccc(F)nc4)CC3)sc2c1 ; Cc1cc(C)c2nc(NC(=O)C3CCN(S(=O)(=O)c4ccc(F)cn4)CC3)sc2c1 ; Cc1cc(C)c2nc(NC(=O)C3CCN(S(=O)(=O)c4ncc(F)cn4)CC3)sc2c1 ; Cc1cc(C)c2nc(NC(=O)C3CCN(S(=O)(=O)c4cccnc4F)CC3)sc2c1 ; Cc1cc(C)c2nc(NC(=O)C3CCN(S(=O)(=O)c4ncccc4F)CC3)sc2c1]

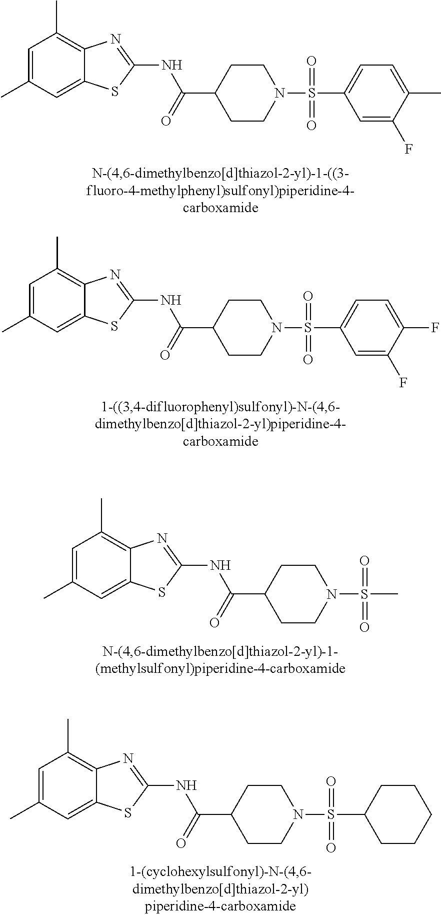

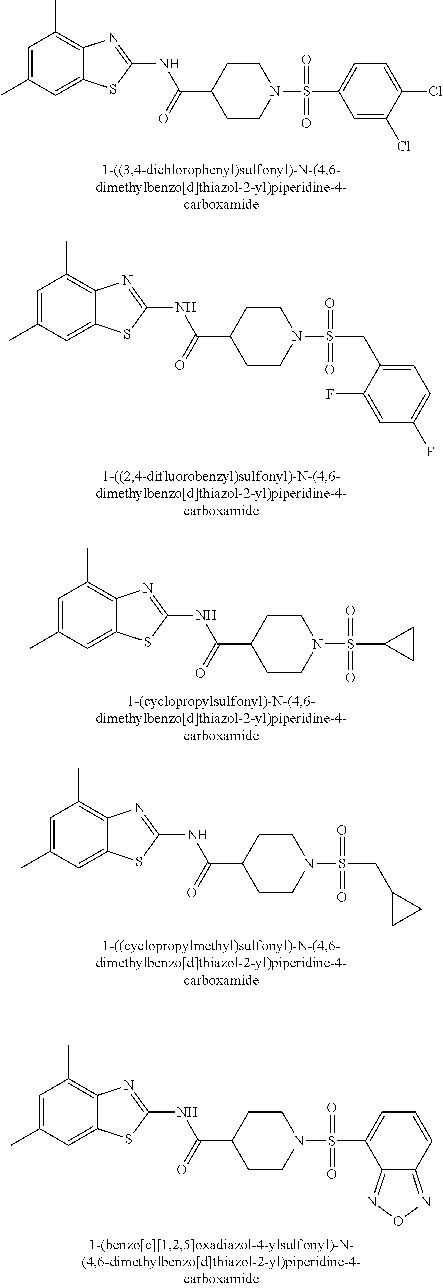 [SMILES: Cc1cc(C)c2nc(NC(=O)C3CCN(S(=O)(=O)c4ccc(Cl)c(Cl)c4)CC3)sc2c1 ; Cc1cc(C)c2nc(NC(=O)C3CCN(S(=O)(=O)Cc4ccc(F)cc4F)CC3)sc2c1 ; Cc1cc(C)c2nc(NC(=O)C3CCN(S(=O)(=O)C4CC4)CC3)sc2c1 ; Cc1cc(C)c2nc(NC(=O)C3CCN(S(=O)(=O)CC4CC4)CC3)sc2c1 ; Cc1cc(C)c2nc(NC(=O)C3CCN(S(=O)(=O)c4cccc5nonc45)CC3)sc2c1]

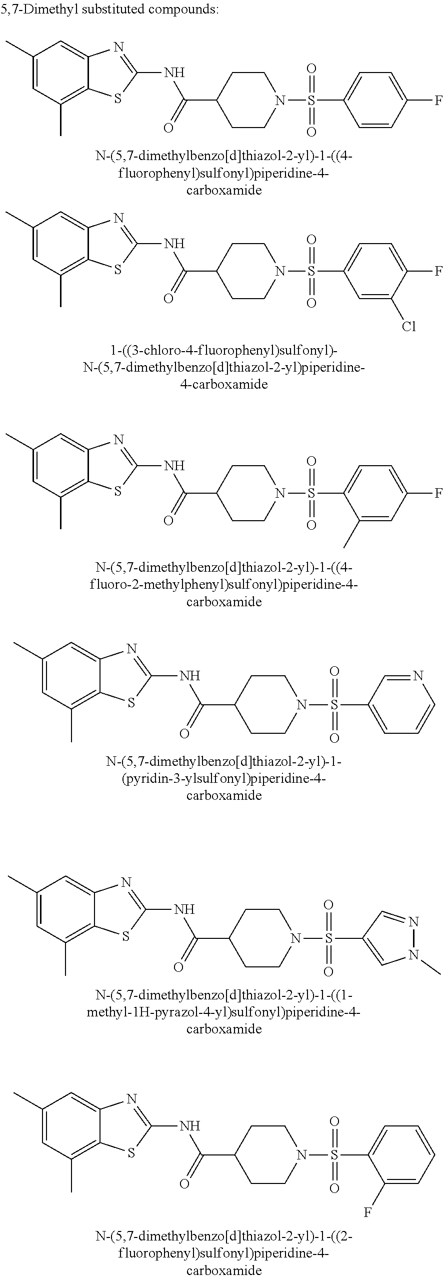

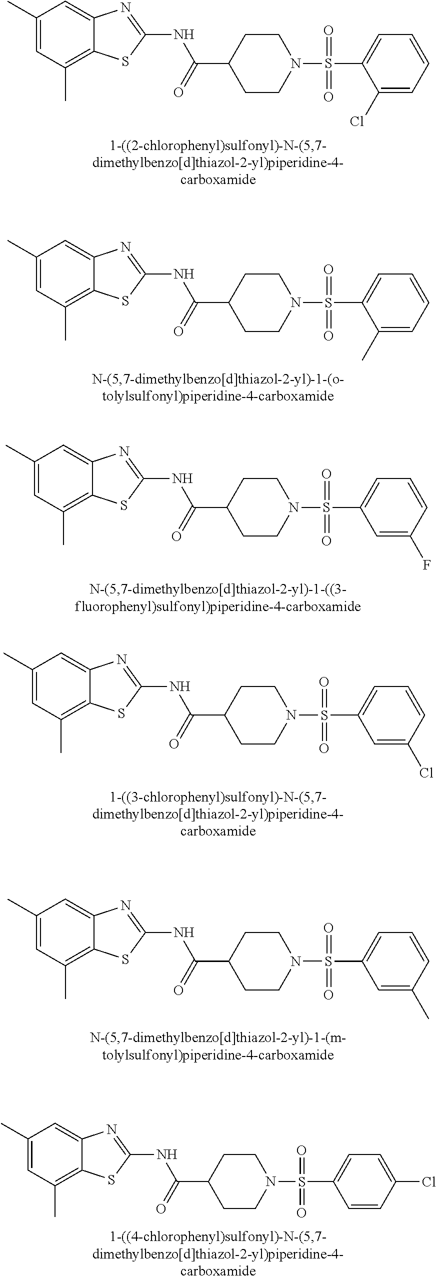

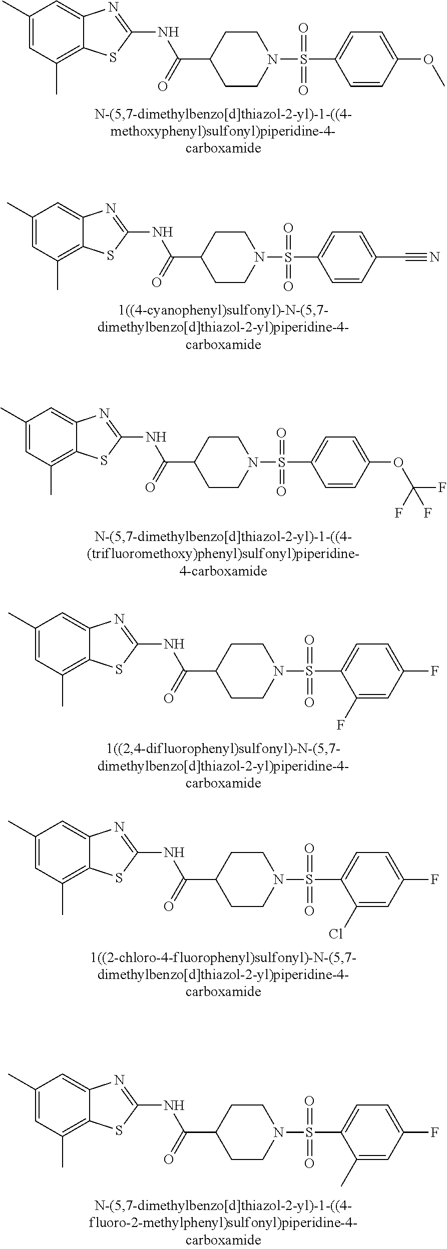

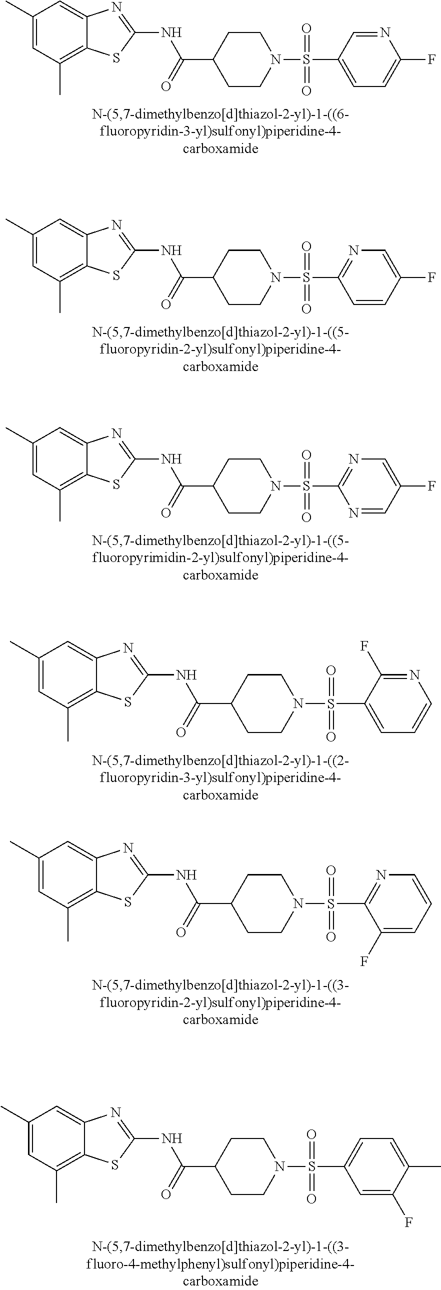

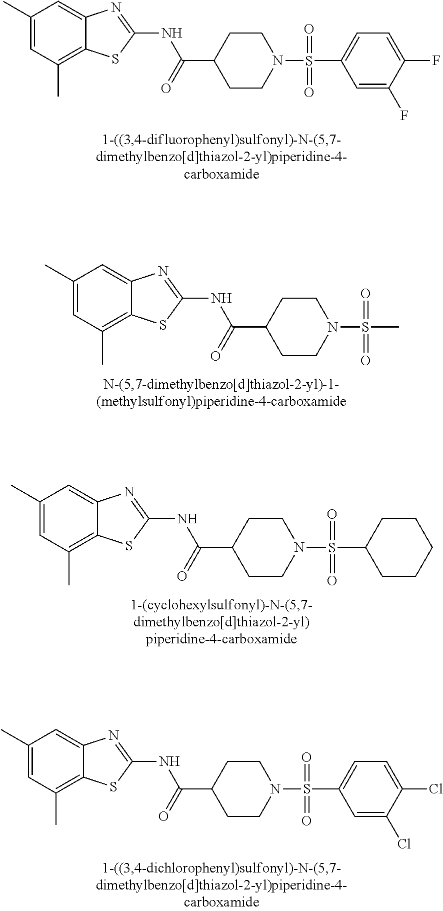

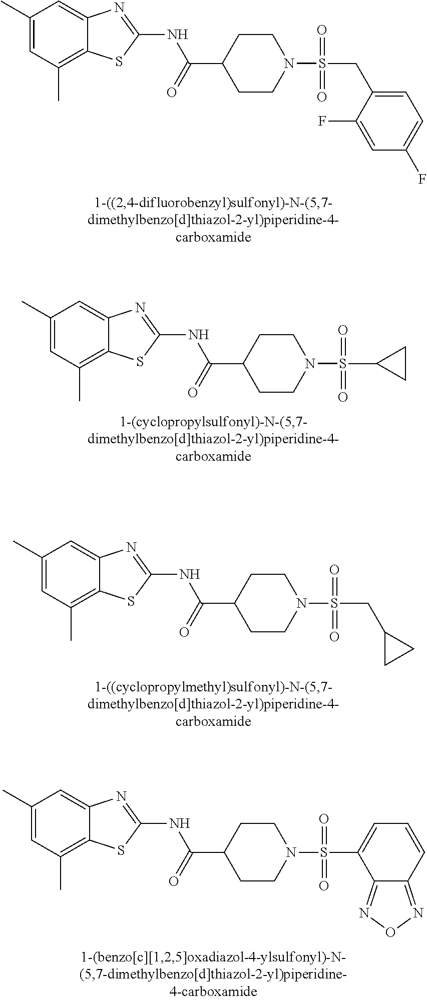 [SMILES: Cc1cc(C)c2sc(NC(=O)C3CCN(S(=O)(=O)Cc4ccc(F)cc4F)CC3)nc2c1 ; Cc1cc(C)c2sc(NC(=O)C3CCN(S(=O)(=O)C4CC4)CC3)nc2c1 ; Cc1cc(C)c2sc(NC(=O)C3CCN(S(=O)(=O)CC4CC4)CC3)nc2c1 ; Cc1cc(C)c2sc(NC(=O)C3CCN(S(=O)(=O)c4cccc5nonc45)CC3)nc2c1]

### NAPE Synthesis

### 1,2-dihexanoyl-sn-glycero-3-phospho-N-oleoyl-ethanolamine (N-oleoyl-PE)

2.5 mL of dry chloroform was added to a 25 mL round-bottom flask. To that, 1,2-dihexanoyl-sn-glycero-3-phosphoethanolamine (164.6 μL, 4 μmol, Avanti Polar Lipids), triethylamine (1.12 μL, 8 μmol), and oleoyl chloride (1.49 μL, 4 μmol, Millipore Sigma) were added. The reaction was stirred for 19 h at room temperature. After the reaction was completed, product was collected using a modified Folch extraction (final extraction solvent composition 6:2:1, chloroform, methanol, saturated NaHCO₃ solution v/v/v). The chloroform (lower) layer was transferred to a glass 10 ml sample tube and dried under N₂ gas. Then, the dried product was dissolved in 2 mL deionized water and 4 mL of ice-cold Folch solution (2:1 chloroform:methanol). This mixture was vortexed for 5 min and incubated on ice for 30 min. The organic and aqueous layers were separated by centrifugation (500×g, 5 min, 4° C.), and the chloroform (lower) layer was saved while the aqueous was discarded. The chloroform layer was dried under gaseous N₂ and re-dissolved in 1 mL of chloroform. This was passed over a Sep-pak plus silica gel cartridge (Waters #WAT036580). The column was washed with 8 mL of 1:9 methanol:chloroform, and then the N-oleoyl-PE eluted with 8 mL of Folch solution. Eluted N-oleoyl-PE was dried under gaseous N₂ and re-dissolved in 800 μL of chloroform. Product was stored in an amber glass vial at −20° C. LC/MS was conducted and recorded on an ThermoFinnigan Quantum electrospray ionization triple quadrupole mass spectrometer in positive ion mode. LCMS calc'd for C₃₅H₆₇NO₉P⁺ [M+H]⁺ 676.5. measured 676.6.

### 1,2-Dioleoyl sn-glycero-3-phospho-N-[²H₄]oleoyl-ethanolamine ([²H₄]N-palmitoyl-PE)

Hydroxybenzotriazole (HOBt) (8.0 mg, 0.052 mmol) and 1-ethyl-3-carbodiimide hydrochloride (EDC-HCl, 10 mg, 0.052 mmol) were added to a solution of 1,2-dioleoyl-sn-3-glycerophosphoethanolamine (25 mg, 0.034 mmol, Avanti Polar Lipids) and 7,7,8,8-d₄-palmitic acid (9.0 mg, 0.035 mmol, Cambridge Isotopes) in CHCl₃ (1 mL). After allowing to react overnight, the reaction mixture was diluted with CHCl₃/MeOH (30 mL) and washed with saturated NH₄Cl (10 mL) and concentrated. The product was purified by column chromatography on silica gel (10% MeOH/CH₂Cl₂) and isolated as a white sticky solid (31 mg, 94%). ¹H NMR (CDCl₃) δ 5.36-5.27 (m, 4H), 5.22-5.18 (m, 1H), 4.37-4.29 (m, 1H), 4.13-4.09 (m, 2H), 3.92-3.90 (m, 3H), 3.50-3.43 (m, 2H), 2.72 (br s, 1H), 2.32-2.24 (m, 5H), 2.20-2.14 (m, 2H), 1.97 (app q, 8H, J=5.9 Hz), 1.62-1.50 (m, 6H), 1.27-1.23 (m, 60H), 0.85 (t, 9H, J=6.5 Hz). LC/MS was conducted and recorded on an ThermoFinnigan Quantum electrospray ionization triple quadrupole mass spectrometer in positive ion mode. LCMS calc'd for C₅₇H₁₀₅D₄NO₉P⁺ [M+H]⁺ 986.8. measured 986.8.

### Example 2

TNFα Expression Studies with Nape-Pld^{-/-} Mice

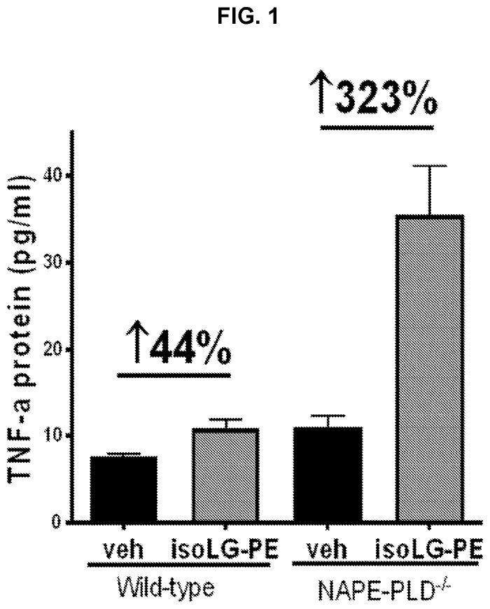

Aldehyde-modified phosphatidylethanolamines (ALPEs) are substrates that induce macrophages and endothelial cells to secrete increased tumor necrosis factor alpha (TNFα). The relationship between NAPE-PLD's phosphodiesterase activity and pro-inflammatory ALPEs, such as isolevuglandin-modified phosphatidylethanolamine (IsoLG-PE) was investigated using bone marrow derived macrophages (BMDMϕs) from Nape-pld^{-/-} mice. The data showed that BMDMϕs from Nape-pld^{-/-} mice have markedly increased TNFα expression in response to IsoLG-PE compared to those from wild-type (WT) mice (FIG. 1). These results indicate that NAPE-PLD inactivates pro-inflammatory ALPEs, such as IsoLG-PE.

### Example 3

### Bioinformatic Analysis

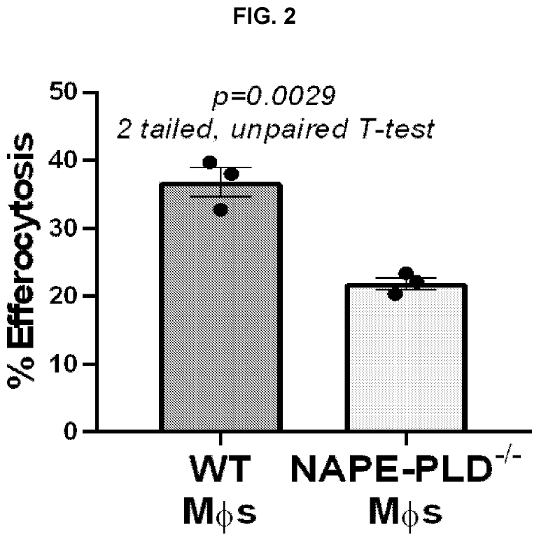

Bioinformatic analysis using the Vanderbilt PrediXcan tool indicated that genetically encoded reduced NAPE-PLD expression increases risk for obesity and type 2 diabetes and diabetic foot and skin ulcers. The reduced capacity of tissue macrophages to carry out efferocytosis (phagocytosis of apoptotic cells) is observed in both diabetic wound ulcers and the necrotic core of unstable atherosclerotic plaques. Additionally, in preliminary studies, it was observed that BMDMϕs from Nape-pld^{-/-} mice had reduced efferocytosis capacity (FIG. 2).

Using cellular expression data (GTex) and the Vanderbilt biobank (BioVU) the Vanderbilt PrediXcan database was used to identify associations between genetically encoded variations in gene expression and the risk for clinical phenotypes. The PrediXcan database was queried for the relationship between reduced NAPE-PLD expression and specific metabolic diseases. The PrediXcan query returned hits including chronic hepatitis (p=7.9×10⁻⁴, r²=0.02, beta=−25.3), type 2 diabetes with ketoacidosis (p=1.5×10⁻³, r²=0.005, beta=−4.5), and dysmetabolic syndrome X (p=4.7×10⁻³, r²=0.003, beta=−2.4). Querying for all disease phenotypes, reduced NAPE-PLD expression was strongly associated with chronic ulcer of leg or foot (p=2.5×10⁻⁷, r²=0.019; beta=−20.4) and chronic ulcer of skin (p=2.3×10⁻⁵, r²=0.019, beta=−12.8).

### Example 4

High-Throughput (HTS) Assay with PED-A1 Probe

Using the fluorogenic NAPE substrate, PED-A1, and recombinant mouse NAPE-PLD (rNAPE-PLD), an in vitro high-throughput (HTS) assay was conducted to screen ˜40,000 compounds for their ability to modulate NAPE-PLD activity. The HTS assay enabled the detection of both NAPE-PLD activators and inhibitors. Over 80 compounds were identified that increased NAPE-PLD activity. Initial structure-activity response studies with the top hits identified benzothiazole phenylsulfonyl-piperidine carboxamide analogs (BT-PSP analogs) as lead compounds for NAPE-PLD activators (Table 1). The lead BT-PSP analog identified, VU534, induces 50% of its activation effect on NAPE-PLD at 270 nM (AC₅₀ 270 nM), and induces a maximal effect of 195% of basal NAPE-PLD activity (Table 1).

TABLE 1
Structure Activity Relationship for Benzothiazole Phenylsulfonyl-Piperidine
Carboxamide (BT-PSP) Analogs

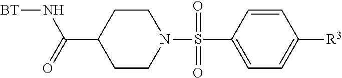

BT = benzothiazolyl moiety

| Compound | BT | R³ | AC₅₀ (nM) | Max % bA (%) | bAC₁₇₅ (nM) |
|---|---|---|---|---|---|
| VU534 | 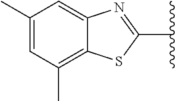 [SMILES: *c1nc2cc(C)cc(C)c2s1] | 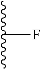 [SMILES: *F] | 270 | 195 | 750 |
| VU536 | 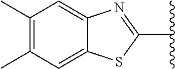 [SMILES: *c1nc2cc(C)c(C)cc2s1] | 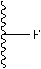 [SMILES: *F] | 1540 | 215 | 2400 |
| VU517 | 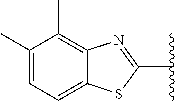 [SMILES: *c1nc2c(C)c(C)ccc2s1] | 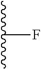 [SMILES: *F] | 767 | 190 | 1480 |
| VU533 | 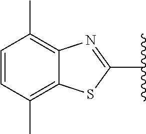 [SMILES: *c1nc2c(C)ccc(C)c2s1] | 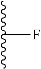 [SMILES: *F] | Not active | <120 | NA |
| VU231 | 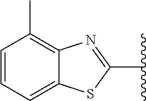 [SMILES: *c1nc2c(C)cccc2s1] | 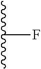 [SMILES: *F] | Not active | <120 | NA |
| VU542 | 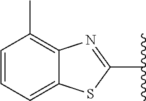 [SMILES: *c1nc2c(C)cccc2s1] | 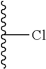 [SMILES: *Cl] | Not active | <120 | NA |
| VU605 | 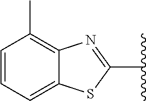 [SMILES: *c1nc2c(C)cccc2s1] | 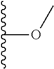 [SMILES: *OC] | 1040 | 170 | NA |
| VU539 | 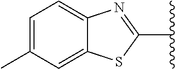 [SMILES: *c1nc2ccc(C)cc2s1] | 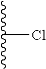 [SMILES: *Cl] | 2380 | 170 | NA |
| VU575 | 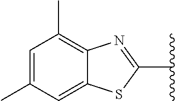 [SMILES: *c1nc2c(C)cc(C)cc2s1] | 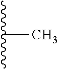 | 950 | 210 | 1690 |

AC₅₀: concentration for 50% maximum activation from basal activity (no activator).
Max % bA: Maximum induced activity compared to basal (100%) activity.
bAC₁₇₅: Compound concentration to induce 175% basal activity.

### Example 5

### Flame-NAPE Fluorescence Probe Design

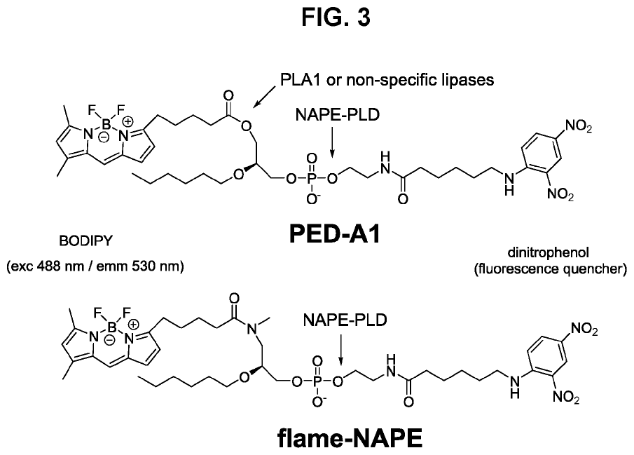

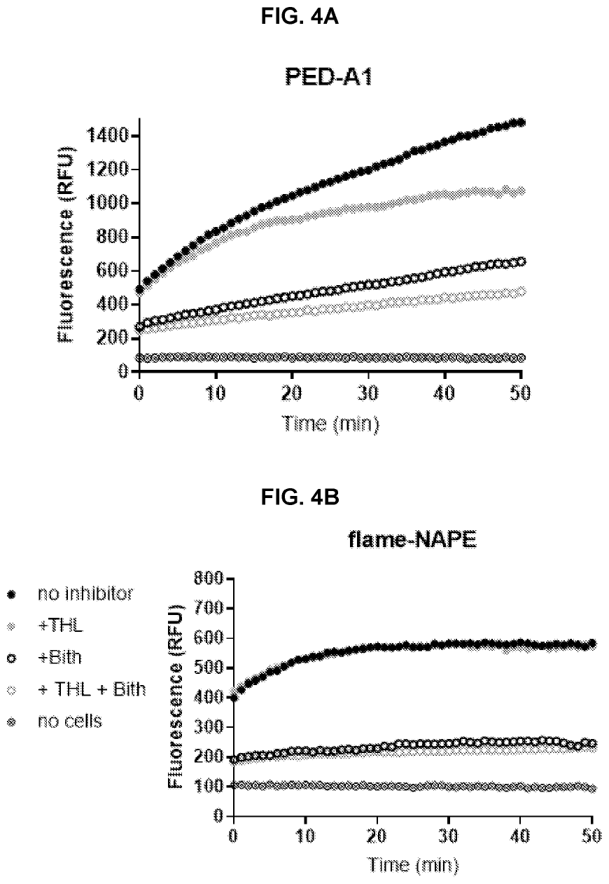

To selectively measure cellular and tissue NAPE-PLD activity fluorogenic amide/ether-NAPE (flame-NAPE), a PED-A1 analog, was developed (FIG. 3). The synthesis and product verification for flame-NAPE were performed by the Molecular Design and Synthesis Center at Vanderbilt University. For flame-NAPE the sn-1 ester bond of the PED-A1 probe was replaced with an N-methyl amide moiety to remove PED-A1's sensitivity to A1-type phospholipases (PLA1s) and other non-specific lipases. HepG2 cells in 96-well plates were treated with 10 μM tetrahydrolipstatin (THL, a pan-lipase inhibitor) and/or 15 μM bithionol (Bith, a NAPE-PLD inhibitor) prior to the addition of either PED-A1 or flame-NAPE. Experimental data showed that phospholipase activity in HepG2 cells measured using flame-NAPE is sensitive to NAPE-PLD inhibition but not PED-A1 inhibition, whereas phospholipase activity measured by PED-A1 is sensitive to both (4 μM) (FIG. 4).

### Example 6

### Efferocytosis Studies

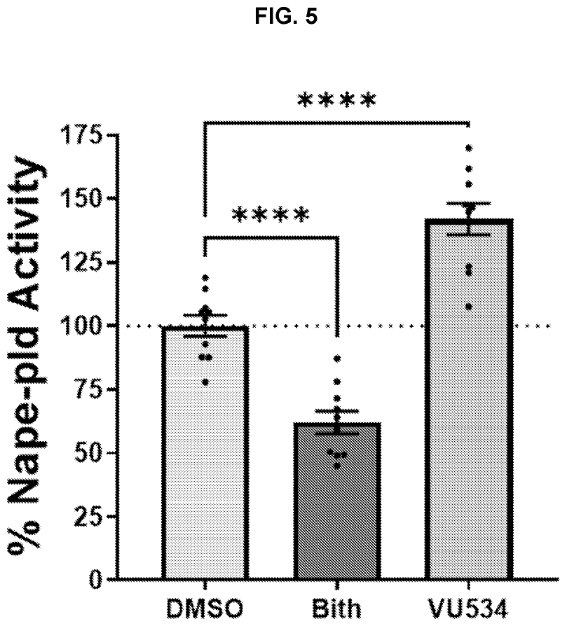

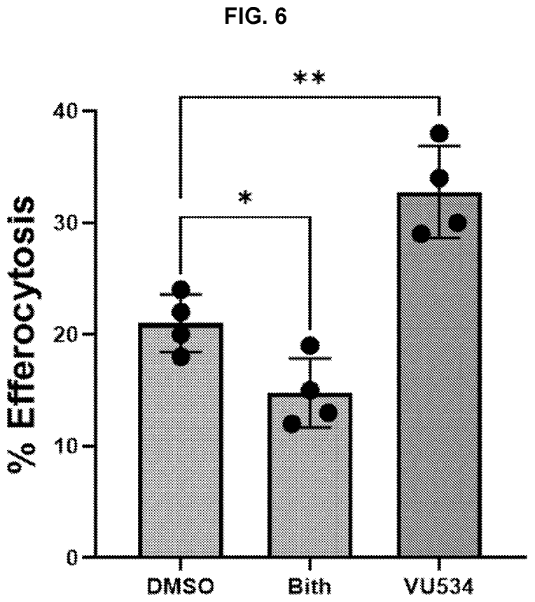

The effects of increased NAPE-PLD activity on efferocytosis were assessed as reduced efferocytosis may contribute to both poor diabetic wound healing and to atherosclerosis. Due to their high MerTK expression, M2c macrophages are 3-fold more effective at efferocytosis than M1 macrophages. Previous studies have demonstrated that inflammatory stimuli (e.g., IFNγ or LPS) reduces NAPE-PLD expression and induces an M1-like phenotype including Tnfα secretion, while treatment with dexamethasone (DEXA) induces an M2c-like phenotype with increased NAPE-PLD expression. Experiments showed that VU534 at 10 μM increased NAPE-PLD activity in primary BMD macrophases (FIG. 5), and thus increased efferocytosis capacity (FIG. 6). Experiments showed that Bith inhibited NAPE-PLD activity, and thus reduced efferocytosis (FIG. 5-6).

### Example 7

### Hepatocyte Cell Line Assays Using Flame-NAPE

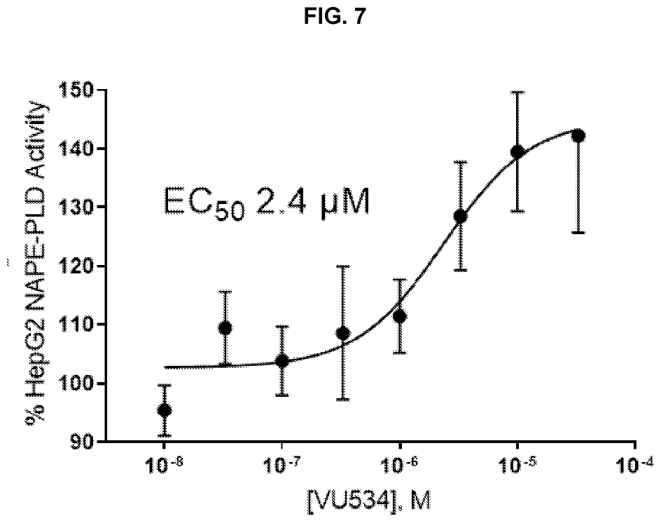

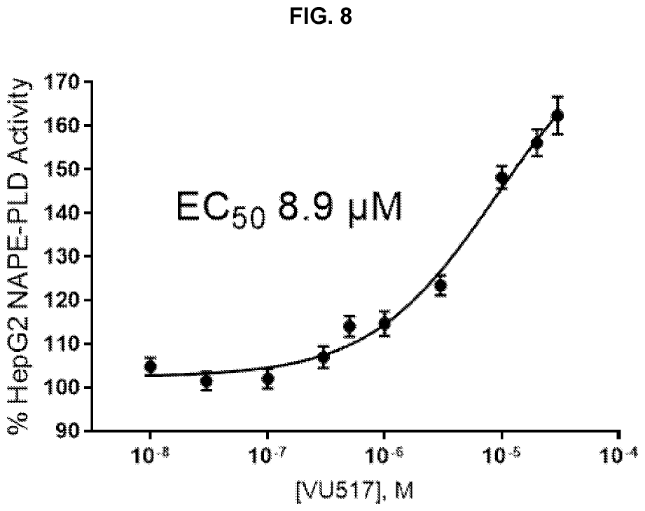

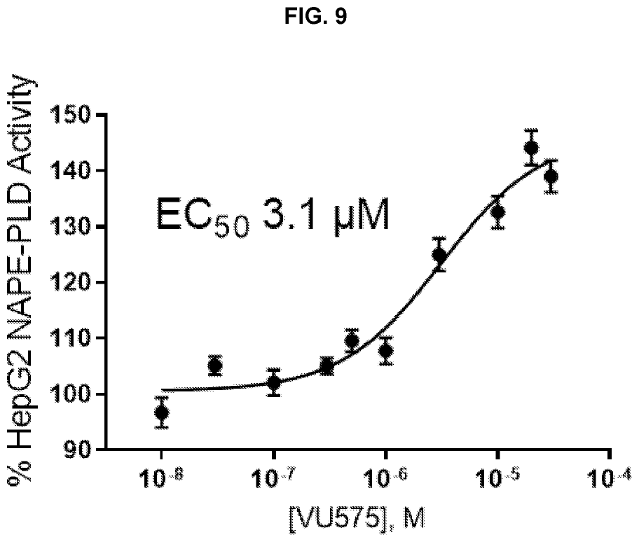

In vitro models of the hepatic steatosis of non-alcoholic fatty liver disease (NAFLD) are well-established, with the primary method being incubation of primary human hepatocytes or hepatocyte cell lines such as HepG2, HuH7, HepaRG, with oleic acid and palmitic acid for 24 to 48 h, then measuring the resulting steatosis, lipid peroxidation, apoptosis, and inflammatory gene expression. Using flame-NAPE to measure NAPE-PLD activity in the human hepatoma HepG2 cell line, the experimental data confirmed that VU534 activated NAPE-PLD in human cells (AC₅₀ 940 nM; max % bA 155%). Bithionol (10 μM) reduced HepG2 flame-NAPE hydrolysis by >67%. Experimental data also showed that VU534 dose-dependently increases NAPE-PLD activity in human hepatocyte HepG2 cells measured with flame-NAPE (EC₅₀ 2.4 μM, Eₘₐₓ 145%, FIG. 7). Additionally, experimental data showed that VU534 analogs such as VU517 and VU575 also dose-dependently increase NAPE-PLD activity in HepG2 cells measured with flame-NAPE (EC₅₀=8.9 μM and 3.1 μM respectively, FIG. 8-9).

### Example 8

### Computational Modeling

To guide the medicinal chemistry campaign, computational modeling of VU534 binding was applied to the solved crystal structure of NAPE-PLD (PDB ID: 4QN9). Three different methods (Maestro SiteFind, ProteinsPlus, and Python-Rx) independently identified a large, druggable pocket in the homodimer interface. The unbiased docking studies produced best scoring poses with VU534 bound in this interface pocket with the sulfonamide moiety making polar contacts to side chains of Arg167, Lys162, Ser152 and the benzothiazole (BT) core moiety and amide carbonyl contacting Ser 151 of the other NAPE-PLD monomer, and the 7-methyl group of the benzothiazole core moiety occupying a small sub-pocket. The computed binding energies for the inactive analog VU533 were higher (i.e., lower affinity) than VU534 (−9.044±0.088 kcal/mol VU533 vs. −9.522±0.101 VU534).

### Example 9

### Michaelis-Menten Studies for VU534

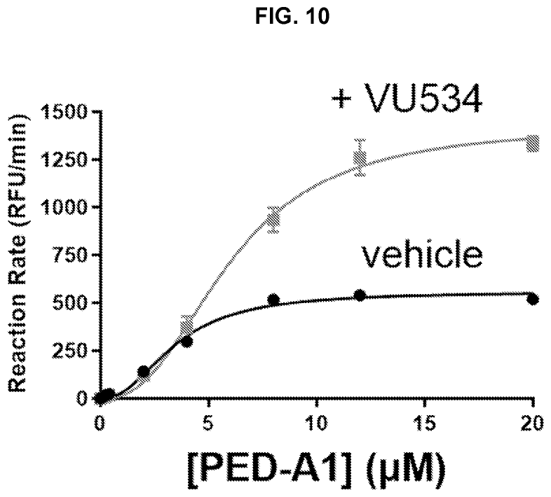

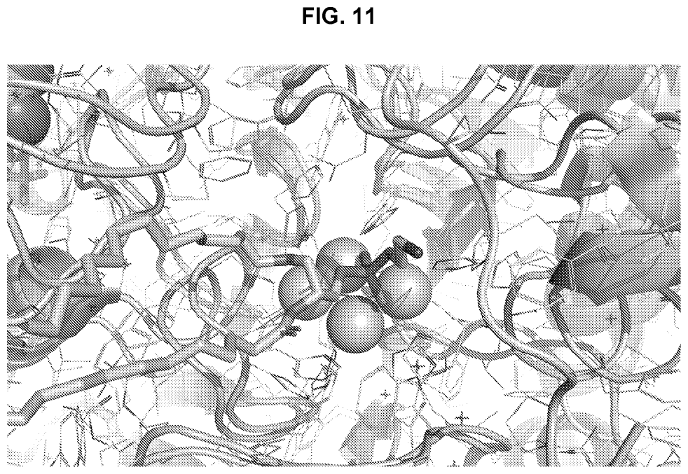

Michaelis-Menten studies demonstrated that VU534 increased the catalytic rate of NAPE-PLD. The data illustrates that VU534 enhanced kcat (turnover number) without reducing the Michaelis-Menten constant, Kₘ (FIG. 10). To further understand this effect, a 50-nanosecond molecular dynamic (MD) simulation was performed using AMBER17. The PDQ9:VU534 complex was prepped for simulation using CHARMM-GUI and ff14SB, TIP3P, GAFF2 force fields were employed for proteins, ligand, and water molecules, respectively. This simulation predicted that VU534 binding to the interface pocket allosterically alters the catalytic site conformation including reorienting the zinc-hydroxide-zinc complex parallel to the substrate (FIG. 11), consistent with the measured increased kcat.

### Example 10

### Concentration Response Curves for FAAH and sEH Inhibition

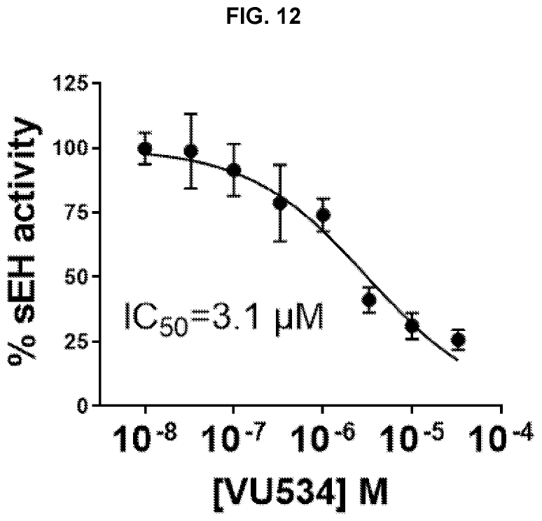

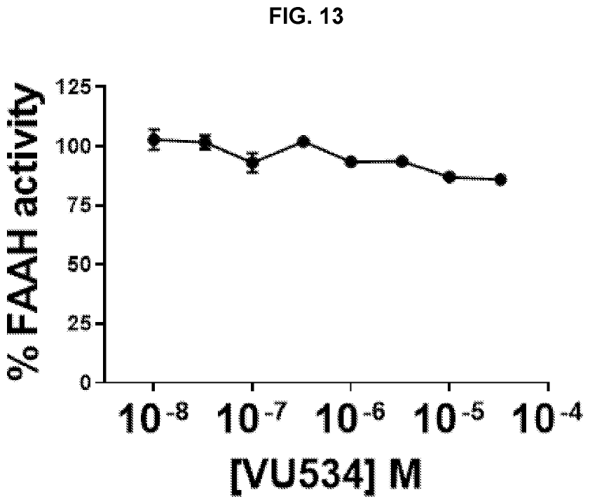

Concentration response curves were plotted for VU534 for the enzymes fatty acid amide hydrolase (FAAH) and soluble epoxide hydrolase (sEH). The concentration response curves for VU534 showed the inhibition of sEH (FIG. 12), and no inhibition of FAAH (even at [VU534]=33 UM, FIG. 13). Additional experiments showed that at 33 μM, several other BT-PSP analogs also inhibited sEH activity >50% (Tables 2 and 3).

TABLE 2
Structure Activity Relationship (SAR) for Benzothiazole Phenylsulfonyl-Piperidine
Carboxamide (BT-PSP) Analogs

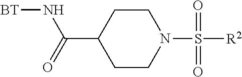

BT = benzothiazolyl moiety

| | | | | | | 10 | | | |
|---|---|---|---|---|---|---|---|---|---|
| | | | | | | μM | | | |
| | | | | | | Mϕ | | | |
| | | | | | | % NA | Hep | | |
| | | | | | 33 μM | PE- | G2 | % FA | % sE |
| | | | | | Mϕ | PLD | EC₅₀ | AH | H |
| Com- | | | Emax | EC₅₀ | % Via- | activ- | (% E | inhi- | inhi- |
| pound | BT | R² | (%) | (μM) | bility | ity | max) | bition | bition |
| VU488 | 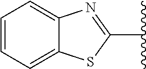 [SMILES: *c1nc2ccccc2s1] | 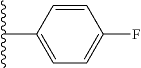 [SMILES: *c1ccc(F)cc1] | 135 | 0.39 | | | | | |
| VU486 | 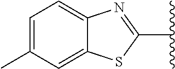 [SMILES: *c1nc2ccc(C)cc2s1] | 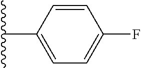 [SMILES: *c1ccc(F)cc1] | 190 | 0.24 | >85 | 105 | | <15 | 40 |
| VU536 | 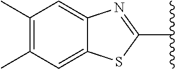 [SMILES: *c1nc2cc(C)c(C)cc2s1] | 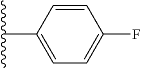 [SMILES: *c1ccc(F)cc1] | 225 | 0.89 | >85 | 155 | | 25 | 50 |
| VU534 | 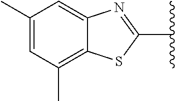 [SMILES: *c1nc2cc(C)cc(C)c2s1] | 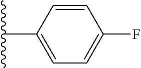 [SMILES: *c1ccc(F)cc1] | 205 | 0.28 | >85 | 140 | 2.4 μM (145%) | <15 | 75 |
| VU533 | 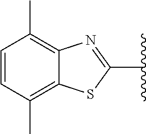 [SMILES: *c1nc2c(C)ccc(C)c2s1] | 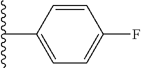 [SMILES: *c1ccc(F)cc1] | <120 | Inactive | | | | | |
| VU517 | 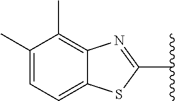 [SMILES: *c1nc2c(C)c(C)ccc2s1] | 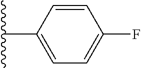 [SMILES: *c1ccc(F)cc1] | 210 | 1.50 | 80 | 125 | 8.9 μM (185%) | <15 | 70 |
| VU231 | 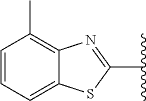 [SMILES: *c1nc2c(C)cccc2s1] | 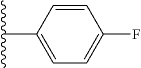 [SMILES: *c1ccc(F)cc1] | <120 | Inactive | | | | | |
| MRJ- 058A | 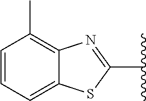 [SMILES: *c1nc2c(C)cccc2s1] | 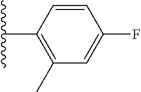 [SMILES: *c1ccc(F)cc1C] | 150 | 2.90 | 55 | | | 20 | 85 |
| MRJ- 058B | 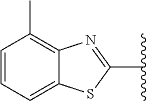 [SMILES: *c1nc2c(C)cccc2s1] | 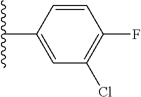 [SMILES: *c1ccc(F)c(Cl)c1] | 180 | 4.70 | >85 | 135 | | <15 | 50 |
| VU542 | 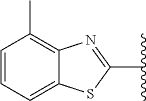 [SMILES: *c1nc2c(C)cccc2s1] | 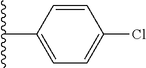 [SMILES: *c1ccc(Cl)cc1] | <120 | Inactive | | | | | |
| VU605 | 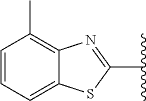 [SMILES: *c1nc2c(C)cccc2s1] | 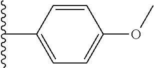 [SMILES: *c1ccc(OC)cc1] | <120 | Inactive | | | | | |
| MRJ- 058C | 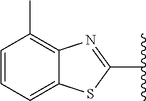 [SMILES: *c1nc2c(C)cccc2s1] | 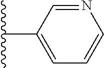 [SMILES: *c1cccnc1] | 175 | 0.94 | >85 | 115 | | <15 | 50 |
| MRJ- 058D | 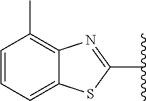 [SMILES: *c1nc2c(C)cccc2s1] | 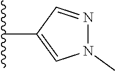 [SMILES: *c1cnn(C)c1] | 240 | 1.50 | >85 | 100 | | <15 | 20 |
| VU539 | 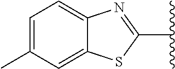 [SMILES: *c1nc2ccc(C)cc2s1] | 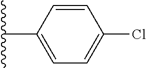 [SMILES: *c1ccc(Cl)cc1] | 170 | 2.38 | | | | | |
| VU575 | 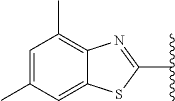 [SMILES: *c1nc2c(C)cc(C)cc2s1] | 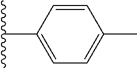 [SMILES: *c1ccc(C)cc1] | 220 | 0.19 | >85 | 105 | 3.1 μM (145%) | <15 | 45 |

Emax (%): Maximal induced activity for recombinant NAPE-PLD compared to basal (100%) activity
EC₅₀: Concentration needed to achieve 50% maximal induced activity in recombinant NAPE-PLD (rNAPE-PLD) activity assay
Mϕ viability: Concentration needed to achieve 50% maximal induced activity in INAPE-PLD activity assay
Mϕ activity: % activity with 10 μM compound compared to vehicle (100%) with 33 μM compound
HepG2 EC₅₀ (Emax): Concentration need to achieve 50% maximal induced activity in HepG2 cells (% maximal induced activity)
% FAAH inhibition: % inhibition compared to vehicle (0%) with 33 μM compound
% sEH inhibition: % inhibition compared to vehicle (0%) with 33 μM compound

TABLE 3
Structure Activity Relationship (SAR) for Benzothiazole Phenylsulfonyl-Piperidine
Carboxamide (BT-PSP) Analogs

| | | | | | In- | | | | |
|---|---|---|---|---|---|---|---|---|---|
| Name | | | | In- | cell | | | | |
| (−1; | | | | cell | NAPE- | | | | |
| HTS | | | | NAPE- | PLD | | | | |
| stock; | | | | PLD | activity | | | | |
| −2; | | | | activity | in | | | | |
| reorder | | | | in | RAW | | | | |
| of | | | | HepG2 | 264.7 | | | | |
| com- | | | In | (10 | (10 | | | | |
| mercial | | | vitro | μM) | μM) | | | | |
| stock; | | | Eₘₐₓ | with | with | | | | |
| −3; | | | (100% | PED- | PED- | | | | |
| syn- | | | activity | A1 | A1 | | | | |
| thesis | | | is | or | or | | % | | |
| by | | | baseline | flame- | flame- | % | viability | Remaining | Remaining |
| VICB | | In | activity | NAPE | NAPE | HepG2 | RAW | FAAH | sEH |
| syn- | | vitro | of | as | as | viability | 264.7 | Activity | Activity |
| thesis | | EC₅₀ | vehicle | sub- | sub- | (33 | (33 | (33 | (33 |
| core) | Structure | (μM) | only) | strate | strate | μM) | μM) | μM) | μM) |
| VU212 | 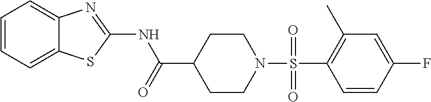 [SMILES: Cc1cc(F)ccc1S(=O)(=O)N1CCC(C(=O)Nc2nc3ccccc3s2)CC1] | | | 197% | | 66% | 32% | 61% | 0% |
| VU233 | 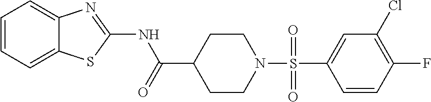 [SMILES: O=C(Nc1nc2ccccc2s1)C1CCN(S(=O)(=O)c2ccc(F)c(Cl)c2)CC1] | | | 130% | | 69% | 111% | 90% | 117% |
| VU203 | 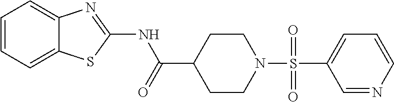 [SMILES: O=C(Nc1nc2ccccc2s1)C1CCN(S(=O)(=O)c2cccnc2)CC1] | 3.4 | 183% | 122% | | 41% | 57% | 79% | 59% |
| VU227 | 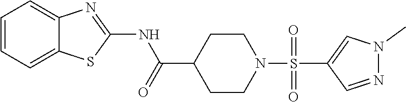 [SMILES: Cn1cc(S(=O)(=O)N2CCC(C(=O)Nc3nc4ccccc4s3)CC2)cn1] | ~2.9 | ~150% | 125% | 102% | 66% | 106% | 97% | 63% |
| VU488 | 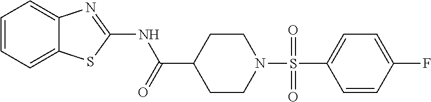 [SMILES: O=C(Nc1nc2ccccc2s1)C1CCN(S(=O)(=O)c2ccc(F)cc2)CC1] | 0.39 | 136% | | | | | | |
| VU541 | 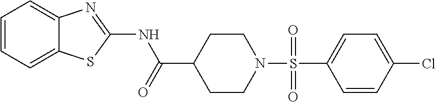 [SMILES: O=C(Nc1nc2ccccc2s1)C1CCN(S(=O)(=O)c2ccc(Cl)cc2)CC1] | 3.3 | 153% | | | | | | |
| VU231 | 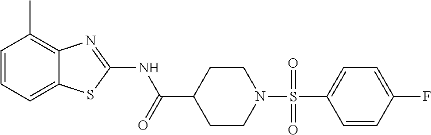 [SMILES: Cc1cccc2sc(NC(=O)C3CCN(S(=O)(=O)c4ccc(F)cc4)CC3)nc12] | 1.1 | 167.8% | | | | | | |
| VU212 | 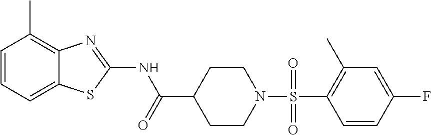 [SMILES: Cc1cc(F)ccc1S(=O)(=O)N1CCC(C(=O)Nc2nc3c(C)cccc3s2)CC1] | ~2.9 | ~150.8% | 268% | | 69% | 56% | 79% | 15% |
| VU209 | 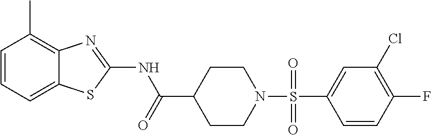 [SMILES: Cc1cccc2sc(NC(=O)C3CCN(S(=O)(=O)c4ccc(F)c(Cl)c4)CC3)nc12] | 4.7 | 179% | 206% | 136% | 73% | 88% | 88% | 62% |
| VU211 | 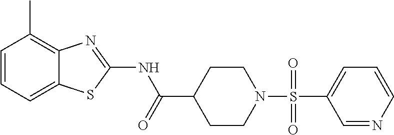 [SMILES: Cc1cccc2sc(NC(=O)C3CCN(S(=O)(=O)c4cccnc4)CC3)nc12] | ~0.94 | ~175.7% | 148% | 114% | 72% | 88% | 86% | 48% |
| VU210 | 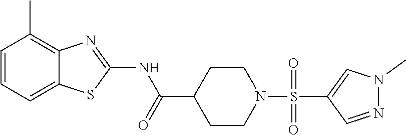 [SMILES: Cc1cccc2sc(NC(=O)C3CCN(S(=O)(=O)c4cnn(C)c4)CC3)nc12] | 1.5 | 239% | 124% | 98% | 67% | 86% | 89% | 81% |
| VU485 | 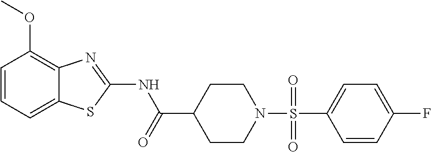 [SMILES: COc1cccc2sc(NC(=O)C3CCN(S(=O)(=O)c4ccc(F)cc4)CC3)nc12] | 29 | 232% | | | | | | |
| VU542 | 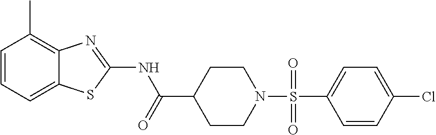 [SMILES: Cc1cccc2sc(NC(=O)C3CCN(S(=O)(=O)c4ccc(Cl)cc4)CC3)nc12] | 1.1 | 182.8% | | | | | | |
| VU605 | 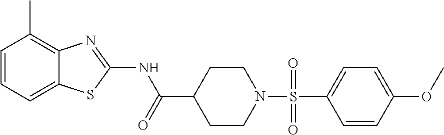 [SMILES: COc1ccc(S(=O)(=O)N2CCC(C(=O)Nc3nc4c(C)cccc4s3)CC2)cc1] | ~0.2 | ~118.8% | | | | | | |
| VU486 | 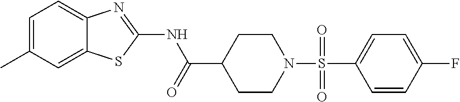 [SMILES: Cc1ccc2nc(NC(=O)C3CCN(S(=O)(=O)c4ccc(F)cc4)CC3)sc2c1] | 0.24 | 189% | | 107% | | 89% | 89% | 60% |
| VU539 | 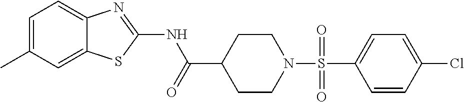 [SMILES: Cc1ccc2nc(NC(=O)C3CCN(S(=O)(=O)c4ccc(Cl)cc4)CC3)sc2c1] | 2.4 | 167% | | | | | | |
| VU601 | 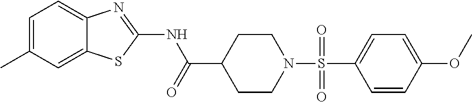 | ~1.0 | 171% | | | | | | |
| VU484 | 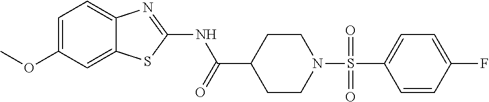 [SMILES: COc1ccc2nc(NC(=O)C3CCN(S(=O)(=O)c4ccc(F)cc4)CC3)sc2c1] | 2.6 | 162% | | | | | | |
| VU572 | 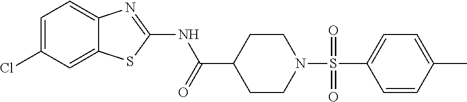 [SMILES: Cc1ccc(S(=O)(=O)N2CCC(C(=O)Nc3nc4ccc(Cl)cc4s3)CC2)cc1] | ~8138 | ~349% | | | | | | |
| VU534 | 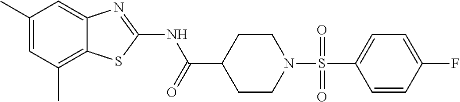 [SMILES: Cc1cc(C)c2sc(NC(=O)C3CCN(S(=O)(=O)c4ccc(F)cc4)CC3)nc2c1] | 0.29 | 209% | 211% | 139% | 91% | 89% | 86% | 27% |
| VU536 | 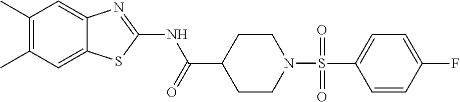 [SMILES: Cc1cc2nc(NC(=O)C3CCN(S(=O)(=O)c4ccc(F)cc4)CC3)sc2cc1C] | 0.89 | 227% | | 155% | | 114% | 75% | 49% |
| VU575 | 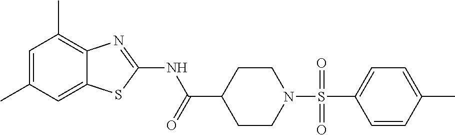 | 0.19 | 215% | | 107% | | 85% | 90% | 55% |
| VU517 | 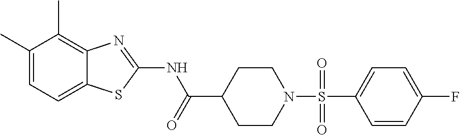 [SMILES: Cc1ccc2sc(NC(=O)C3CCN(S(=O)(=O)c4ccc(F)cc4)CC3)nc2c1C] | 1.5 | 213% | | 123% | | 83% | 90% | 29% |

### Example 10

### Effects of Various BT-PSP Analogs (10 μM) on NAPE-PLD Activity of RAW264.7 Macrophages

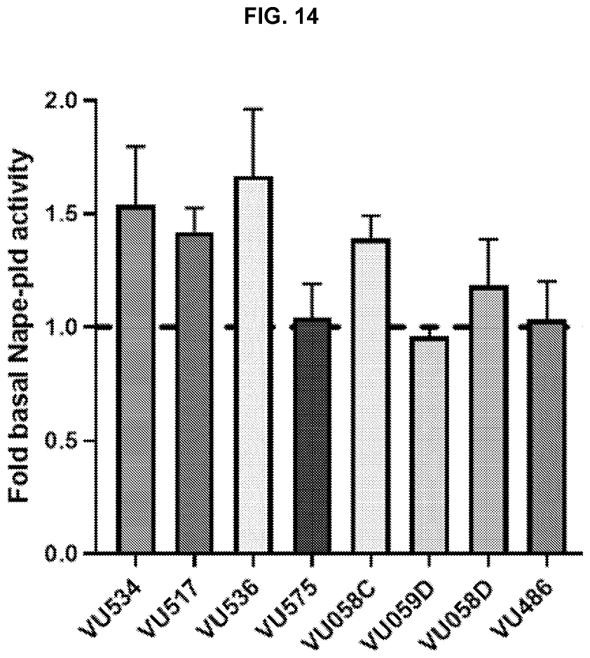

FIG. 14 shows the effects of various BT-PSP Analogs (10 μM) on NAPE-PLD activity of RAW264.7 macrophages.

### Example 11

### Materials

Initial stocks of potential NAPE-PLD modulator compounds were purchased from Life Chemicals and provided by the Vanderbilt HTS screening facility. Additional compounds were synthesized by the Vanderbilt Chemical Synthesis core. LEI-401, [²H₄]PEA and [²H₄]OEA were purchased from Cayman Chemicals. N-palmitoyl-PE, 1,2-dioleoyl-PE and 1,2-dihexanoyl-PE were purchased from Avanti Polar Lipids. PED-A1 was purchased from Invitrogen. Flame-NAPE was synthesized as previously described. See Zarrow et al., J. Lipid Res. 63(1): 100156 (2022). [²H₄]N-palmitoyl-PE was synthesized using [²H₄] palmitic acid (Cambridge Isotope Laboratories) and 1,2-dioleoyl-PE and N-oleoyl-PE was synthesized using 1,2-dihexanoyl-PE and oleoyl chloride (Millipore Sigma). Recombinant mouse NAPE-PLD with a C-terminal hexahistidine tag was expressed in E. coli and purified using cobalt affinity beads as previously described. See Aggarwal et al., J. Biol. Chem. 295(21): 7289-7300 (2020). The expression plasmid including the full-length human NAPEPLD gene with a C-terminal hexahistidine tag inserted in a pET plasmid was purchased from VectorBuilder, and the protein expressed and purified in an identical manner to recombinant mouse NAPE-PLD. The sEH inhibitors TPPU (N-[1-(1-Oxopropyl)-4-piperidinyl]-N′-[4-(trifluoromethoxy)phenyl]urea) and AUDA (12-[[(tricyclo[3.3.1.13,7]dec-1-ylamino)carbonyl]amino]-dodecanoic acid) were purchased from Cayman Chemical and Sigma Chemicals, respectively.

Biochemical NAPE-PLD Assays with Recombinant NAPE-PLD

In vitro fluorescence NAPE-PLD activity assays using recombinant NAPE-PLD and either PED-A1 or flame-NAPE as fluorogenic substrate were performed as previously described except with small modifications as noted below. See Aggarwal et al., J. Biol. Chem. 295(21): 7289-7300 (2020); Zarrow et al., J. Lipid Res. 63(1): 100156 (2022).

For the HTS assays, test compounds were incubated with recombinant enzyme for 1 h prior to adding PED-A1 (final 0.4 μM) mixed with N-palmitoyl-dioleoyl-PE (final 3.6 μM) to adjust for the high sensitivity of the Panoptic instrument (WaveFront Biosciences). Assays used black-wall, clear-bottom, non-sterile, and non-treated 384-well plates (Greiner Bio-One 781906). The assay was read in kinetic fluorescence mode on the Panoptic instrument for 4 min and the slope of the signal from 30-100 s was used for analysis. A total of 39,328 compounds from the Vanderbilt Discovery Collection were tested, each at 10 μM. The tested compounds were chosen to represent a structurally diverse selection from the full library. Each 384-well plate included 320 test compound wells and 64 control wells. Unlike previous pilot screening assays, bithionol (10 μM final) was used in place of lithocholic acid (100 μM final) as the inhibitor control. Before performing high-throughput screening, a checkerboard assay was performed to validate the assay parameters. This yielded a Z′ score of 0.676. Z′ scores were also calculated for each plate during screening, and plates with scores <0.5 were re-run. The average Z′ across all screening plates was 0.52, and the total hit rate was 3.6%. B-scores were calculated from the initial slopes across each plate using WaveGuide software (WaveFront Biosiences). Modulator hits were defined as compounds with absolute B-scores of 3 or higher. The number of compounds in various B-score ranges were as follows: 21 to −10, 12 compounds; −10 to −5, 221 compounds; −5 to −3, 770 compounds; −3 to 3, 37924 compounds; 3 to 4, 314 compounds; 4 to 6, 70 compounds; 6 to 10, 15 compounds. Activator hits with B-scores of >3 were selected for the replication assay and a selection of analogs of the activators.

To identify false hits that modulated fluorescence of the BODIPY moiety indirectly, potential hit compounds were incubated with BODIPY-FL C5, a BODIPY-labeled free fatty acid, and measured the effect on fluorescence. One compound directly modulated fluorescence in the absence of enzyme and was therefore eliminated from further evaluation.

Concentration response curve (CRC) experiments used the same assay conditions as the HTS assay, except that graded concentrations of each test compound were used, with total amount of vehicle (DMSO) kept constant. CRC experiments with purified recombinant mouse NAPE-PLD were performed on two separate days, with values from each day normalized to vehicle only controls on same plate, and then all normalized values from both days averaged together. CRC experiments with human NAPE-PLD represent value from only a single day, due to limited amounts of this recombinant enzyme.

A similar in vitro fluorescence Nape-pld activity assay was used for LEI-401 and VU233 competition assays except that this assay was performed using black-walled clear-bottom 96-well plates with 3.5 μM PED-A1 and no N-palmitoyl-dioleoyl-PE used as substrate and read in a BioTek Synergy H1 plate reader with the slope of the fluorescence signal from 0-4 min (linear phase) used as the assay readout. LEI-401, VU233, and VU534 were incubated for 1 h prior to addition of PED-A1. The Michaelis-Menten study used this same assay except with graded concentration of flame-NAPE.

For LC/MS assays, N-oleoyl-PE was added as substrate after 1 h pre-incubation with compound VU534, VU533, VU233 or vehicle. 90 min after N-oleoyl-PE was added, the reaction was quenched by adding 3 volumes of ice-cold methanol containing [²H₄]OEA and [²H₄]N-palmitoyl-PE and then 6 volumes of ice-cold chloroform.33 The lower phase was dried under nitrogen gas and dissolved in 100 μL mobile phase A. High performance liquid chromatography was performed using a 2.1 mm C18 guard column (Phenomenex AJ0-8782), and a rapid gradient ramp. Mobile phase A was 5:1:4 (v/v/v) isopropanol: methanol: water, with 0.2% v/v formic acid, 0.66 mM ammonium formate and 3 μM phosphoric acid included as additives. Mobile phase B was 0.2% (v/v) formic acid in isopropanol. Initial column conditions were 5% mobile phase B, followed by gradient ramp to 95% B over 0.5 min, held at 95% B for 2 min, the returned to initial conditions (5% B) over 1 min. Flow rate throughout was 100 μL/min. Injection volume was 2 μL. The sample injector needle was washed before each injection using a strong wash of methanol, and a weak wash of 1:1:1:1 (v/v/v/V) isopropanol: methanol: acetonitrile: water, with 0.2% formic acid, 0.3 mM ammonium formate, and 0.37 mM phosphoric acid included as additives. Multiple reaction monitoring for the following ions were monitored: OEA [M+H]⁺: m/z 326.3→m/z 62.1; [²H₄]OEA [M+H]⁺: m/z 330.3→m/z 66.1; N-oleoyl-PE [M+NH₄]⁺: m/z 693.5→m/z 308.3 (quantifier), m/z 693.5→m/z 271.2 (qualifier); [²H₄]N-palmitoyl-PE [M+NH₄]⁺: m/z 1003.8→m/z 286.3 (quantifier), m/z 1003.8→m/z 603.5 (qualifier). The ratio of peak height for OEA to [²H₄]OEA was used to calculate to amount of OEA generated and the ratio of peak height for N-oleoyl-PE to [²H₄]N-palmitoyl-PE was used to calculate the amount of N-oleoyl-PE remaining. These values were then used to calculate the OEA/N-oleoyl-PE ratio.

### Other Biochemical Assays

The fluorescence interference assay was performed using the same method as the HTS assay, but with BODIPY-FL C5 (Thermo Fisher Scientific) used in place of PED-A1. sEH and FAAH activity assays were performed according to the manufacturer's specifications (Cayman Chemicals).

### Cell-Based NAPE-PLD Assays

NAPE-PLD activity was measured in cells as previously described, except that FluoroBrite DMEM (Gibco A1896701) was used as media. See Zarrow et al., J. Lipid Res. 63(1): 100156 (2022). For RAW264.7 assays, PED-A1 (3.6 μM final) was used as the substrate with 10 μM orlistat added to inhibit PLA1 activity, while for HepG2 cells, flame-NAPE was used (with no orlistat added).

Cytotoxicity was measured using MTT as previously described except that the studies used 96-well plates with 100 μL of 0.3 mg/mL MTT solution was added after 24 h of treatment and then replaced after 3 h with 0.1 M HCl in isopropanol. See Aggarwal et al., J. Biol. Chem. 295(21): 7289-7300 (2020). Viability was expressed as percent absorbance at 560 nm relative to vehicle controls.

### Efferocytosis Assays

Male C57BL6/j wild-type or Napepld^{-/-} mice were euthanized with isoflurane and hind legs were removed. Marrow was flushed from femurs and tibias using DMEM containing 4.5 g/L glucose and a 26-gauge needle. Cell suspensions were passed over a 40-μm filter, centrifuged at 500×g, and resuspended in 50 ml of DMEM containing 4.5 g/L glucose, 20% L-cell conditioned media, 10% heat-inactivated FBS, and 1% penicillin/streptomycin. 10 ml of cell suspension was plated into each of five 100-mm dishes and incubated for four days at 37° C. and 5% CO₂. On day four, non-adherent cells and debris were aspirated from the plates and replaced with fresh media. After 7 days of differentiation, cells were harvested for use in experiments.

Assays were performed according to previously described protocols. See e.g., Doran et al. J. Clin. Invest. 127(11): 4075-4089 (2017); Proto et al., Immunity 49(4): 666-677 (2018). Bone marrow-derived macrophages were seeded at 0.25×10⁶ cells/well in a non-tissue culture-treated 24-well plate and allowed to adhere overnight. Macrophages were treated with various compounds at a final concentration of 10 μM or DMSO as a vehicle for 6 hours prior to each experiment. Jurkat cells were exposed to UV light (254 nm) for 5 minutes to induce apoptosis and then incubated in a 37° C. incubator with 5% CO₂ for 2 hours. Surveillance staining of these cells routinely yielded approximately 80-90% apoptosis (Annexin V+) using this method. Apoptotic Jurkat cells were labeled with either CellVue Claret (Millipore Sigma) or Cell Trace Violet (Invitrogen) per the manufacturer's instructions. After staining, cells were resuspended in macrophage medium at a density of 0.75×10⁶ cells/mL and 500 μL of this suspension was added to the drug-containing media on the macrophages to achieve a cell ratio of 3:1 Jurkats: macrophages. After incubating for 45 minutes at 37° C. and 5% CO₂, the medium was aspirated, and the macrophages were gently washed twice with PBS to remove unbound apoptotic cells. Macrophages were then removed from the plate using Cell Stripper (Sigma), washed, resuspended in staining buffer consisting of 2% FBS in PBS with 2 mM EDTA, and blocked with anti-mouse CD16/32 antibodies for 15 minutes on ice. After blocking, cells were pelleted and resuspended with F4/80. Cells were incubated for 45 minutes on ice in the dark, then washed and resuspended in staining buffer for analysis. Cells were analyzed using an Attune NxT cytometer (Thermo Fisher) and data were analyzed using FlowJo software to quantify the proportion of F4/80⁺ macrophages that co-stained for apoptotic cells (% efferocytosis).

### Statistical Analyses

All statistical analyses and non-linear regression analyses were performed using GraphPad Prism 9 software, except for calculation of B-scores for high throughput screening assays, which were calculated using WaveGuide software (WaveFront Biosiences).

### Example 12

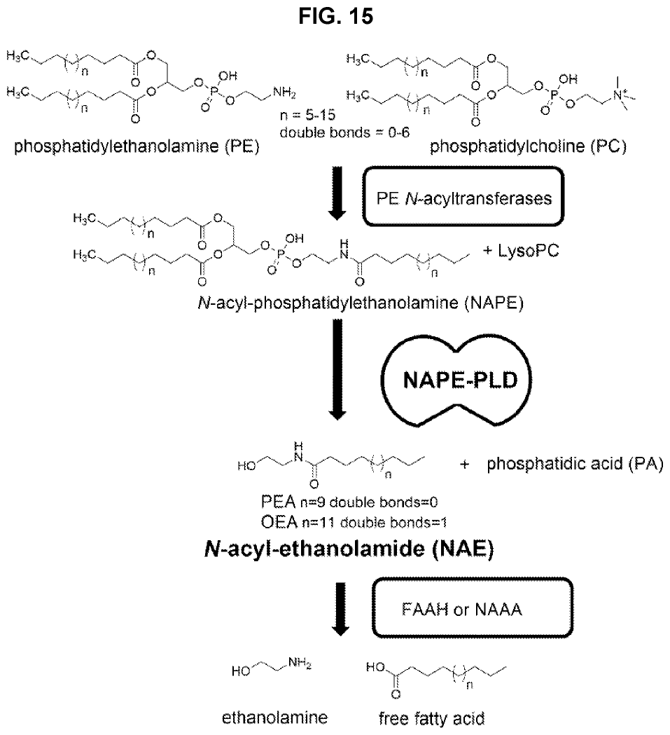

NAPE-PLD is a zinc metallohydrolase within the metallo-β-lactamase superfamily. NAPE-PLD hydrolyzes N-acyl-phosphatidylethanolamines (NAPEs) to phosphatidic acid and N-acyl-ethanolamides (NAEs) such as palmitoylethanolamide (PEA) and oleoylethanolamide (OEA) 4-5 (FIG. 15). In rodents, high-fat or Western diets decrease Napepld expression and levels of PEA and OEA in a variety of tissues. NAPEPLD expression is also reduced in atherosclerotic plaques (especially unstable plaques) of human coronary arteries. In mice, directly administering NAEs or NAE-boosting bacteria counteract atherosclerosis as well as other cardiometabolic diseases including obesity, glucose intolerance, and non-alcoholic fatty liver disease. Importantly, these treatments inhibit enlargement of the necrotic core within atherosclerotic lesions. Cellular studies show that PEA and OEA enhance M2 polarization and the efferocytosis capacity of bone-marrow derived macrophages (BMDM) via GPR55 and/or PPARα dependent mechanisms. Together these studies suggest that reduced macrophage NAPEPLD expression could lead to reduced efferocytosis by macrophages and thereby drive expansion of the necrotic core and atherosclerosis.

If NAPE-PLD regulates efferocytosis, then small molecules that enhance macrophage enzyme activity should enhance macrophage efferocytosis and could therefore potentially inhibit the development of unstable atherosclerotic lesions. While several small molecule inhibitors of NAPE-PLD have been reported, there are currently no small molecule activators of NAPE-PLD. The goal was to identify small molecules that could enhance macrophage enzyme activity in order to test their effects on the macrophage efferocytosis capacity.

### New NAPE-PLD Activator Chemotype Identified by HTS and Early SAR Studies

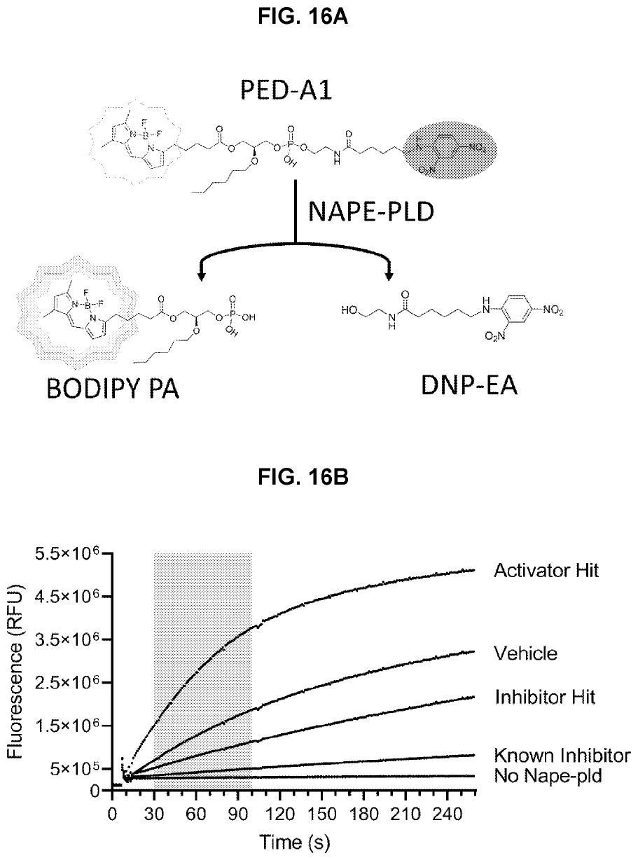

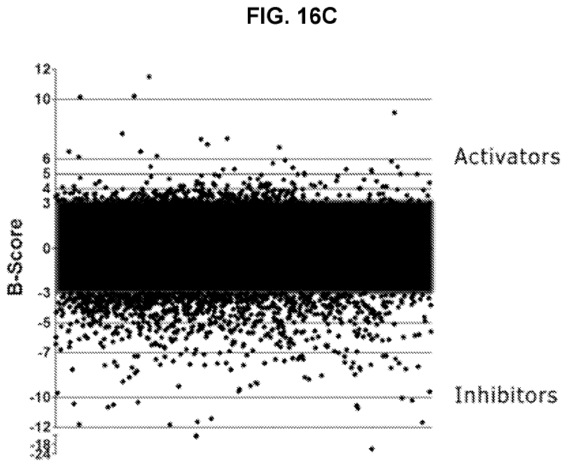

This study screened 39,328 compounds from the Vanderbilt Discovery Collection, a chemical library of lead-like compounds, for their effects on Nape-pld activity using the commercially available fluorogenic NAPE analog, PED-A1 (FIG. 16A) and recombinant mouse Nape-pld. The change in the measured rate of fluorescence after addition of commercially available PED-A1 (FIG. 16B) was used to calculate a B-score for each compound (FIG. 16C). From the 39,238 compounds screened, 399 were judged as potential activators based on observed change in fluorescence by at least 3 standard deviations compared to vehicle.

Three of the 399 identified activators (Table 4, Entries 1-3) shared a common benzothiazole phenylsulfonyl-piperidine carboxamide (BT-PSP) core structure. To determine if this series of benzothiazoles might serve as a tractable lead for the development of activators as chemical probes, additional structurally similar compounds were obtained using a combination of targeted purchasing of commercial molecules (Table 4, Entries 4-13) and discrete chemical synthesis (Table 4, Entries 14-22). The activity of this series of 22 benzothiazole phenylsulfonyl-piperidine carboxamides thus obtained was assessed for enzyme activation using recombinant mouse NAPE-PLD (Table 4). Compounds VU534 and VU533 (entries 8 and 9) proved the most potent of the series of enzyme activators, both showing half-maximal activation concentrations (EC₅₀) of 0.30 μM and a more than two-fold maximal induction of NAPE-PLD activity relative to vehicle controls (Eₘₐₓ>2.0). Nine other BT-PSPs (Entries 3, 5-7, 10-14) had EC₅₀≤1.1 μM and Eₘₐₓ>1.7. The remaining members of the series had EC₅₀<10 μM and Emax>1.7, except that compound VU212 (Entry 16) showed poor efficacy (Eₘₐₓ<1.6), and VU205 and VU233 proved inactive (Eₘₐₓ<1.2). With this preliminary structure-activity relationship (SAR) data, VU534 and VU533 were selected as chemical probes for study of NAPE-PLD activation and VU233 as a negative control in studies outlined below.

Table 4 shows in vitro NAPE-PLD modulation by benzothiazole phenylsulfonyl-piperidine carboxamides (BT-PSPs). EC₅₀ represent concentration (in μM) required for half-maximal activity, expressed in μM. Eₘₐₓ represents maximal increase in activity (as fold activity of vehicle control).

TABLE 4
Preliminary SAR of NAPE-PLD activators^{a}

| | | | EC₅₀ μM^{b} | Eₘₐₓ^{c} (95% |
|---|---|---|---|---|
| Entry | Structure | VU Number | (95% Cl) | Cl) |
| 1 | 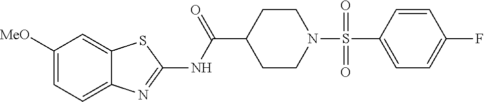 [SMILES: COc1ccc2nc(NC(=O)C3CCN(S(=O)(=O)c4ccc(F)cc4)CC3)sc2c1] | VU484 | 2.3 (1.8-3.1) | 2.1 (2.0-2.2) |
| 2 | 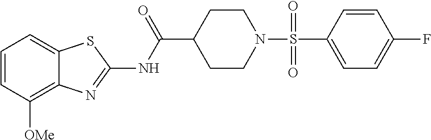 [SMILES: COc1cccc2sc(NC(=O)C3CCN(S(=O)(=O)c4ccc(F)cc4)CC3)nc12] | VU485 | 12.0 (7.3-85.9) | 2.0 (1.8-4.0) |
| 3 | 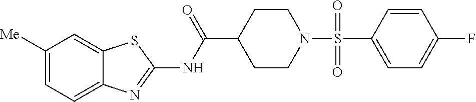 [SMILES: Cc1ccc2nc(NC(=O)C3CCN(S(=O)(=O)c4ccc(F)cc4)CC3)sc2c1] | VU486 | 0.32 (0.15-0.58) | 1.8 (1.7-1.9) |
| 4 | 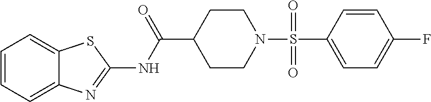 [SMILES: O=C(Nc1nc2ccccc2s1)C1CCN(S(=O)(=O)c2ccc(F)cc2)CC1] | VU488 | 3.3 (2.7-4.2) | 1.7 (1.6-1.8) |
| 5 | 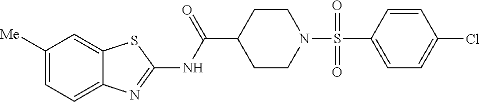 [SMILES: Cc1ccc2nc(NC(=O)C3CCN(S(=O)(=O)c4ccc(Cl)cc4)CC3)sc2c1] | VU539 | 0.74 (0.46-1.1) | 1.7 (1.6-1.8) |
| 6 | 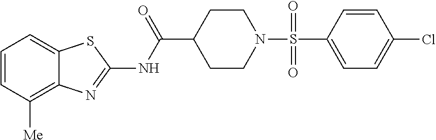 [SMILES: Cc1cccc2sc(NC(=O)C3CCN(S(=O)(=O)c4ccc(Cl)cc4)CC3)nc12] | VU542 | 0.95 (0.66-1.36) | 1.9 (1.9-2.0) |
| 7 | 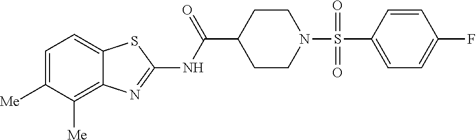 [SMILES: Cc1ccc2sc(NC(=O)C3CCN(S(=O)(=O)c4ccc(F)cc4)CC3)nc2c1C] | VU517 | 1.1 (0.9-1.4) | 2.0 (2.0-2.1) |
| 8 | 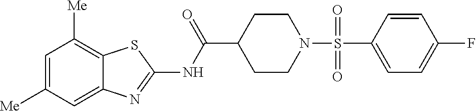 [SMILES: Cc1cc(C)c2sc(NC(=O)C3CCN(S(=O)(=O)c4ccc(F)cc4)CC3)nc2c1] | VU534 | 0.30 (0.17-0.47) | 2.1 (2.0-2.2) |
| 9 | 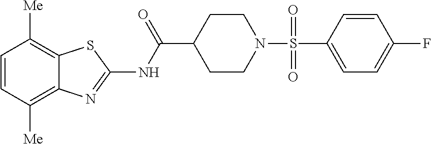 [SMILES: Cc1ccc(C)c2sc(NC(=O)C3CCN(S(=O)(=O)c4ccc(F)cc4)CC3)nc12] | VU533 | 0.30 (0.16-0.44) | 2.6 (2.6-2.8) |
| 10 | 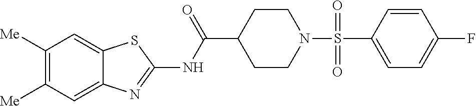 [SMILES: Cc1cc2nc(NC(=O)C3CCN(S(=O)(=O)c4ccc(F)cc4)CC3)sc2cc1C] | VU536 | 1.1 (0.7-1.6) | 2.3 (2.2-2.4) |
| 11 | 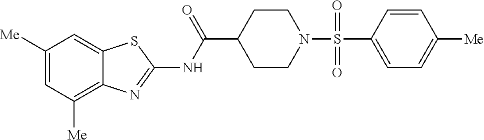 [SMILES: Cc1ccc(S(=O)(=O)N2CCC(C(=O)Nc3nc4c(C)cc(C)cc4s3)CC2)cc1] | VU575 | 0.57 (0.34-0.92) | 2.4 (2.3-2.5) |
| 12 | 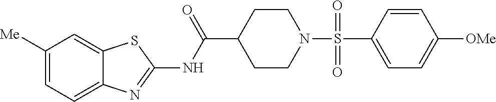 [SMILES: COc1ccc(S(=O)(=O)N2CCC(C(=O)Nc3nc4ccc(C)cc4s3)CC2)cc1] | VU0601 | 0.94 (0.71-1.2) | 1.8 (1.8-1.9) |
| 13 | 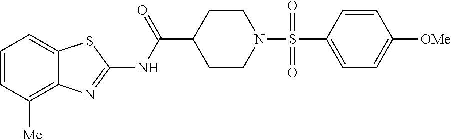 [SMILES: COc1ccc(S(=O)(=O)N2CCC(C(=O)Nc3nc4c(C)cccc4s3)CC2)cc1] | VU605 | 0.74 (0.50-1.22) | 2.1 (2.0-2.2) |
| 14 | 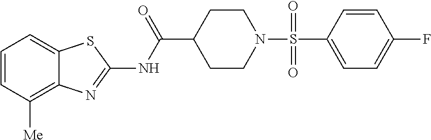 [SMILES: Cc1cccc2sc(NC(=O)C3CCN(S(=O)(=O)c4ccc(F)cc4)CC3)nc12] | VU231 | 1.1^{d} | 2.0^{d} |
| 15 | 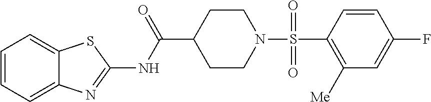 [SMILES: Cc1cc(F)ccc1S(=O)(=O)N1CCC(C(=O)Nc2nc3ccccc3s2)CC1] | VU205 | NA^{e} | <1.2 |
| 16 | 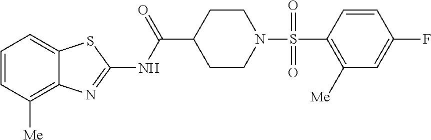 [SMILES: Cc1cc(F)ccc1S(=O)(=O)N1CCC(C(=O)Nc2nc3c(C)cccc3s2)CC1] | VU212 | 2.5 (1.8-3.3) | 1.6 (1.5-1.6) |
| 17 | 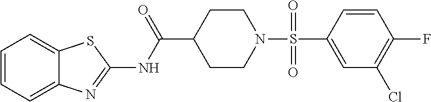 [SMILES: O=C(Nc1nc2ccccc2s1)C1CCN(S(=O)(=O)c2ccc(F)c(Cl)c2)CC1] | VU233 | NA^{e} | <1.2 |
| 18 | 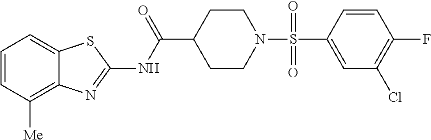 [SMILES: Cc1cccc2sc(NC(=O)C3CCN(S(=O)(=O)c4ccc(F)c(Cl)c4)CC3)nc12] | VU209 | 3.5 (2.4-5.4) | 1.9 (1.8-2.1) |
| 19 | 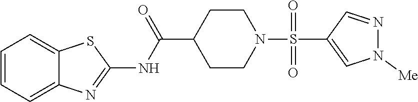 [SMILES: Cn1cc(S(=O)(=O)N2CCC(C(=O)Nc3nc4ccccc4s3)CC2)cn1] | VU227 | 4.1 (2.6-9.1) | 1.7 (1.6-1.9) |
| 20 | 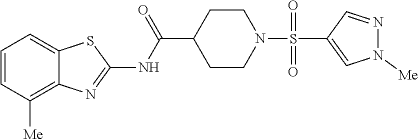 [SMILES: Cc1cccc2sc(NC(=O)C3CCN(S(=O)(=O)c4cnn(C)c4)CC3)nc12] | VU210 | 1.8 (1.3-2.3) | 2.4 (2.3-2.6) |
| 21 | 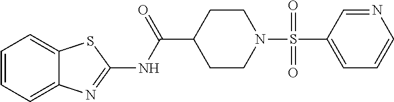 [SMILES: O=C(Nc1nc2ccccc2s1)C1CCN(S(=O)(=O)c2cccnc2)CC1] | VU203 | 4.8 (3.2-7.4) | 1.8 (1.7-2.0) |
| 22 | 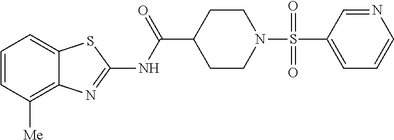 [SMILES: Cc1cccc2sc(NC(=O)C3CCN(S(=O)(=O)c4cccnc4)CC3)nc12] | VU211 | 1.4 (1.0-1.9) | 2.1 (2.0-2.2) |

^{a}Compounds were either purchased from commercial source or prepared in house by chemical synthesis.
^{b}EC₅₀: concentration needed for 50% maximal activator effect.
^{c}Eₘₐₓ: maximal efficacy expressed as fold increase in NAPE-PLD activity versus vehicle only.
^{d}95% Cl for EC₅₀ and Eₘₐₓ indeterminant due to poor fit.
^{e}NA: not active.

### Utility as Probes of NAPE-PLD Activation in Mouse and Human Cells

To determine if the selected small molecules could be used as probes of NAPE-PLD activation in cultured cells, their cytotoxicity was evaluated in RAW264.7 mouse macrophages and HepG2 human hepatocytoma cells. Graded concentrations of VU534 showed minimal cytotoxicity up to 30 μM in either cell line. Likewise, VU533 and VU233 also showed no cytotoxicity when tested at 30 μM in either RAW264.7 or HepG2 cells.

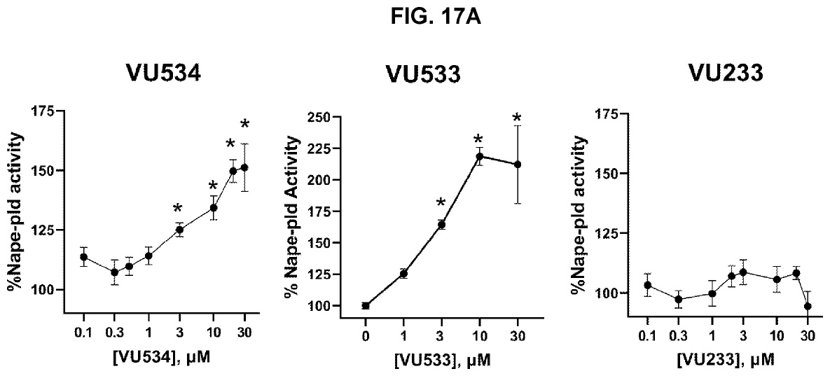

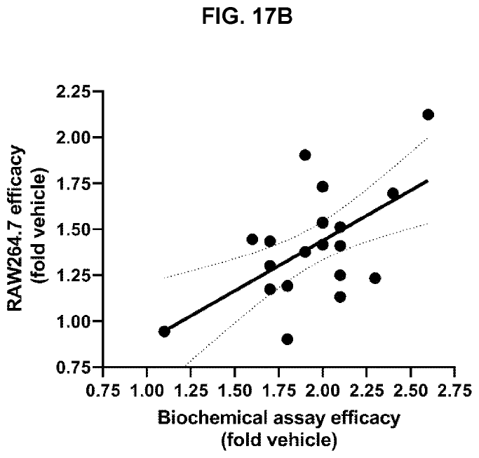

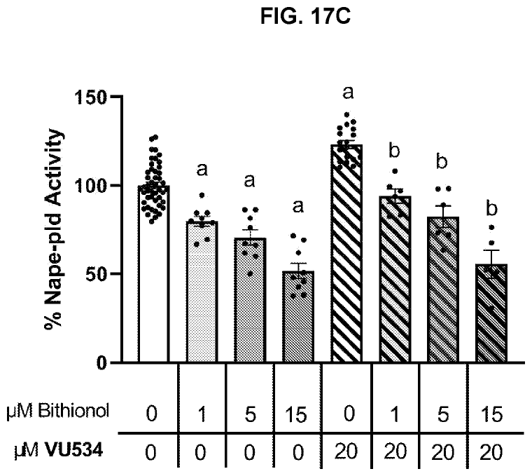

Next the efficacy of activators VU534 and VU533 and inactive VU233 were examined for the ability to increase NAPE-PLD activity in RAW264.7 cells. Both activators VU534 and VU533 significantly increased NAPE-PLD activity, while VU233 showed no significant effect (FIG. 17A). Other analogs in the series of 22 benzothiazole phenylsulfonyl-piperidine carboxamides that increased the activity of recombinant NAPE-PLD in the biochemical assay (Table 4) also significantly increased NAPE-PLD activity in RAW264.7 cells in a concentration-dependent manner, with a correlation observed between the efficacy in the biochemical assay and their efficacy in RAW264.7 cells (FIG. 17B). To confirm that the effect seen in RAW264.7 cells was due to NAPE-PLD modulation, the effects of bithionol, a known irreversible inhibitor of NAPE-PLD14 was tested. The increase in RAW264.7 cellular NAPE-PLD activity induced by 20 μM VU534 was blocked in a concentration-dependent manner by bithionol (FIG. 17C). These results are consistent with enzyme activation of VU534 on PED-A1 hydrolysis being dependent on Nape-pld rather than an alternate modulating pathway.

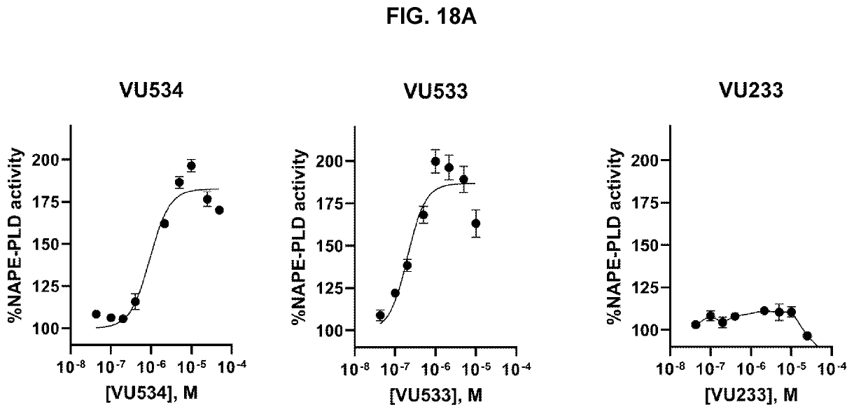

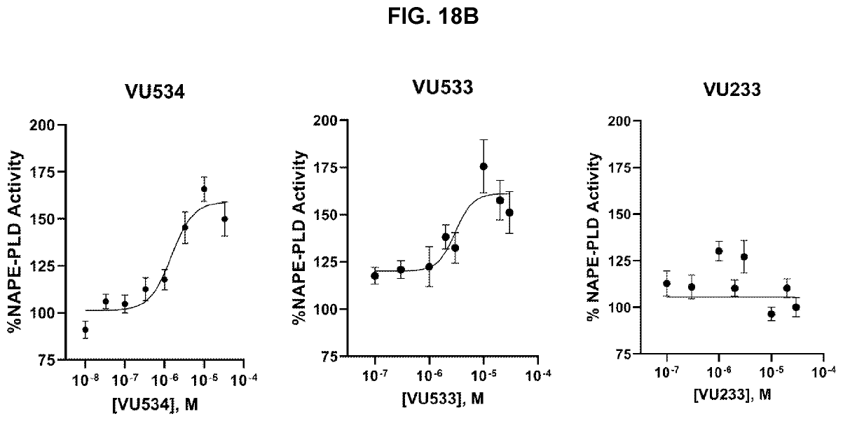

The effects of NAPE-PLD modulators were then determined using purified human NAPE-PLD and human-derived culture cells. Both VU534 and VU533 invoked concentration-dependent increases in the activity of recombinant human NAPE-PLD, while the inactive VU233 had no significant effect (FIG. 18A). The potency of VU534 and VU533 for activating human NAPE-PLD was somewhat less than for activating mouse NAPE-PLD (Table 4). Other benzothiazoles (Table 4) also activated recombinant human NAPE-PLD. VU534 and VU533, but not the inactive compound VU233, increased NAPE-PLD activity in HepG2 cells in a concentration-dependent manner (FIG. 18B).

### Biochemical Characterization of Lead NAPE-PLD Activators

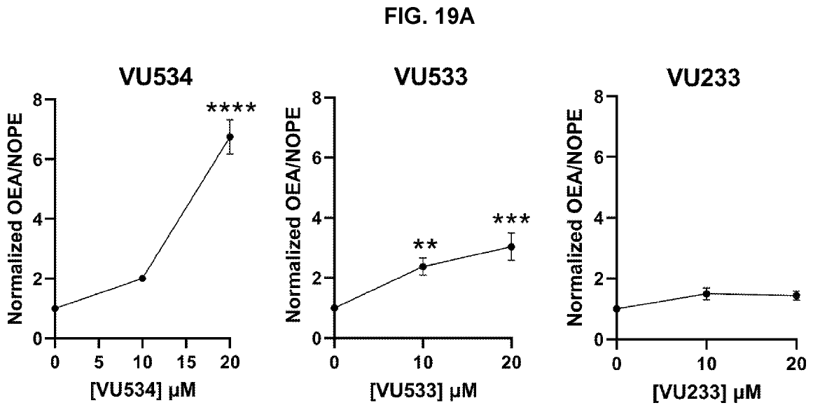

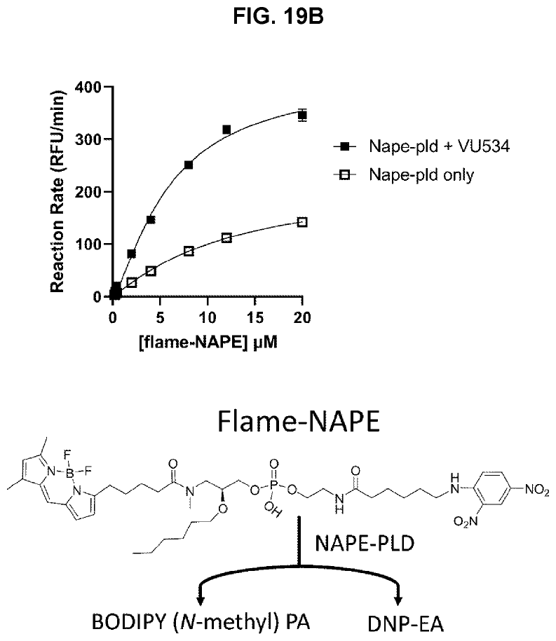

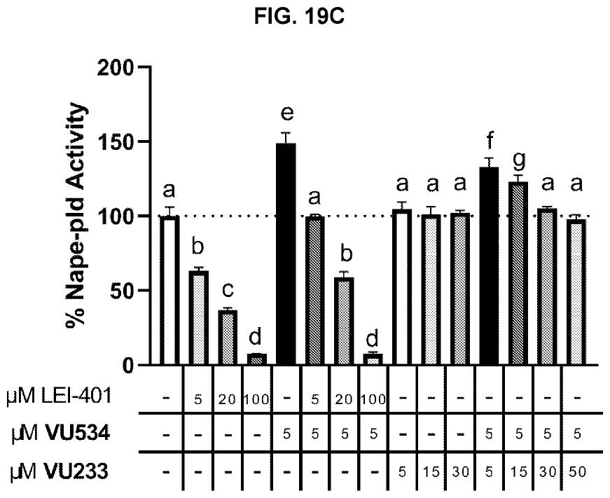

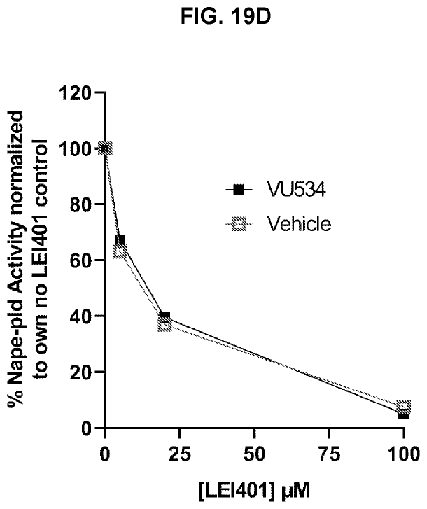

To confirm the results of the fluorescence-based assays, an orthogonal biochemical Nape-pld assay based on LC/MS was employed. Recombinant mouse NAPE-PLD was pre-treated with VU534, VU533, VU233 or vehicle for 30 min, then N-oleoyl-phosphatidylethanolamine (NOPE) was added for 90 min, and the resulting levels of OEA and NOPE was measured by LC/MS. Both activators VU534 and VU533 significantly increased the OEA/NOPE ratio, while inactive VU233 had no significant effect (FIG. 19A). To further characterize the effect of VU534 on NAPE-PLD activity, a Michaelis-Menten analysis was performed using recombinant mouse NAPE-PLD and graded concentrations of flame-NAPE, a selective NAPE-PLD fluorogenic substrate resistant to competing esterase activity. Compound VU534 lowered the K_{1/2} (12.4 μM with vehicle vs. 5.9 μM with VU534) and increased maximal velocity (227 RFU/min with vehicle vs 421 RFU/min with VU534) of NAPE-PLD (FIG. 19B).

These results were consistent with VU534 leading to NAPE-PLD activation by way of allosteric modulation. The effect of LEI-401, a known, reversible inhibitor of NAPE-PLD, was examined or the inactive VU233 on the ability of VU534 to enhance Nape-pld activity. LEI-401 inhibited mouse NAPE-PLD activity in a concentration-dependent manner, with a concentration of 100 μM achieving nearly complete inhibition (FIG. 19C). In the absence of LEI-401, 5 μM VU534 increased Nape-pld activity 1.5-fold. When 5 μM of compound VU534 was added, LEI-401 still inhibited Nape-pld activity in a concentration-dependent manner, with 100 μM of LEI-401 again being sufficient for near complete effect. Although the absolute Nape-pld activity was higher in the presence of VU534 than without it, when activity was normalized as percentage of initial activity, the extent of inhibition by LEI-401 was not significantly different (FIG. 19D), suggesting that VU534 does not compete with LEI-401 for the same binding site. Further insight into the mode of enzyme activation was obtained when inactive VU233 was observed to suppress the enzyme activation of 5 μM VU534 in a concentration-dependent manner (FIG. 19C). These results are consistent with inactive VU233 being a neutral allosteric binder that binds to the same site as VU534.

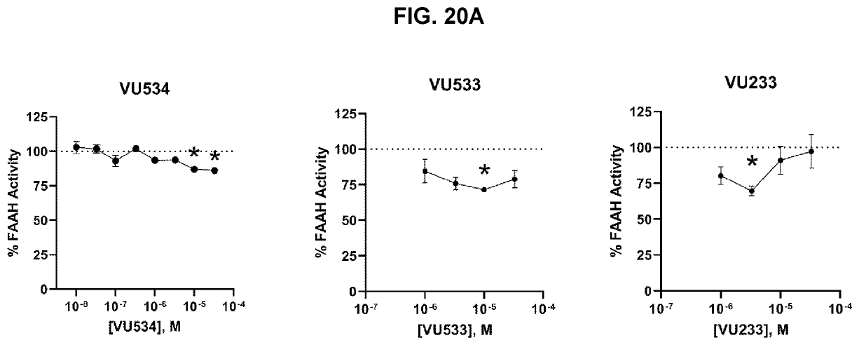

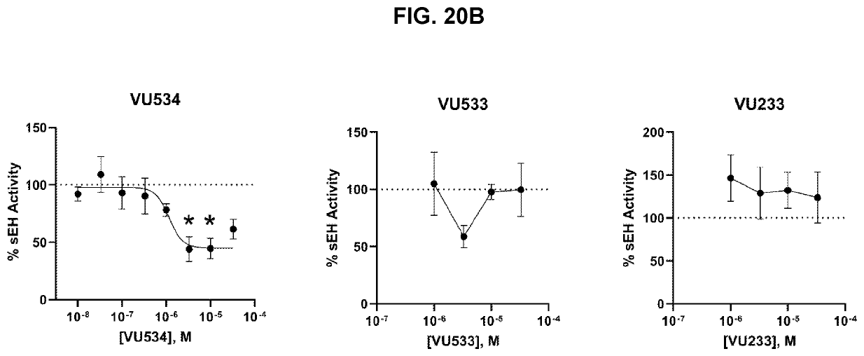

A survey of the literature revealed that a series of benzothiazoles with structural features somewhat similar to VU534 and VU533 have been developed as dual inhibitors of fatty acid amide hydrolase (FAAH) and soluble epoxide hydrolase (sEH). Because inhibition of FAAH would increase cellular NAE levels independently of NAPE-PLD (FIG. 15), the activators were tested to determine whether they modulated FAAH activity. Graded concentrations of VU534, VU533, and VU233 showed only weak inhibition of FAAH activity (FIG. 20A). The activators were tested to determine whether they modulated sEH activity. While sEH does not lie in the biochemical pathway for NAE biosynthesis or metabolism, inhibition, or genetic ablation of sEH increases the levels of epoxy fatty acids and thereby exerts biological effects similar to the known effects of NAEs such as reducing obesity, cardiovascular disease, pain, and inflammation. Graded concentrations of VU534 showed modest inhibition of sEH (IC₅₀ 1.2 μM, 95% CI 0.5-2.4 μM, maximal inhibition 55%), while neither VU533 nor VU233 significantly inhibited seH (FIG. 20B). Other compounds of this series showed variable effects on FAAH and sEH activity. These modest off-target effects should not significantly interfere with the use of these compounds to probe the contribution of NAPE-PLD activity as long as bona fide sEH inhibitors are also tested as controls.

### Modulation of NAPE-PLD Activity Modulates Efferocytosis by Macrophages

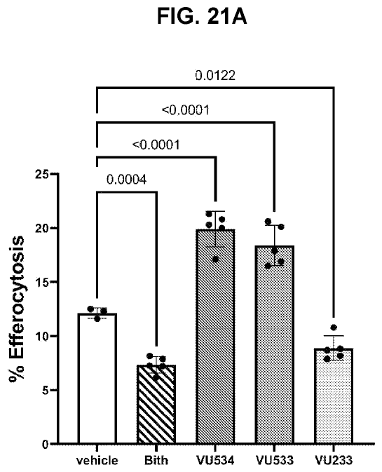

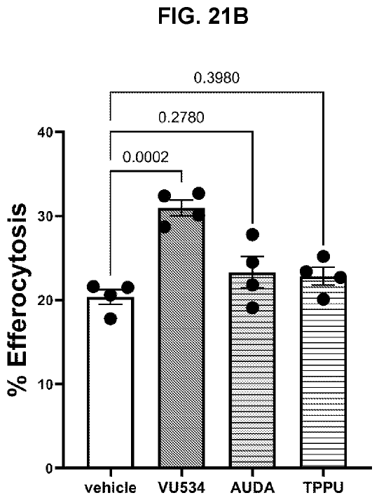

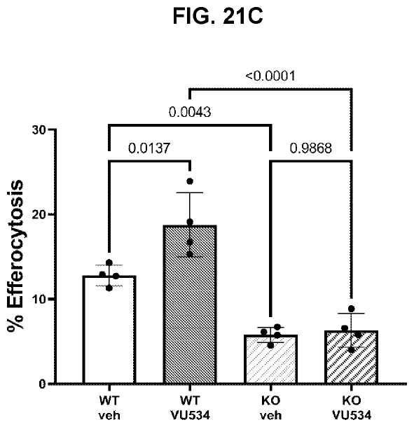

Rinne et al. showed that PEA, a NAPE-PLD product, enhanced the ability of bone-marrow derived macrophages (BMDM) to carry out efferocytosis. See Rinne et al., Arterioscler. Thromb. Vasc. Biol. 38(11): 2562-2575 (2018). The effects of NAPE-PLD modulation were assessed on efferocytosis by BMDM. Treatment of BMDM with 10 μM of NAPE-PLD inhibitor bithionol (Bith) markedly reduced efferocytosis compared to vehicle treated BMDM, while treatment with 10 μM of either NAPE-PLD activators VU534 or VU533 significantly enhanced efferocytosis. (FIG. 21A). In contrast, treatment with 10 μM compound VU233 modestly reduced efferocytosis. Given the modest inhibitory effects of compound VU534 on sEH, the effect of two bona fide sEH inhibitors, AUDA and TPPU, on efferocytosis was examined. Neither AUDA nor TPPU significantly enhanced efferocytosis (FIG. 21B), indicating that sEH inhibition was not responsible for the effect of VU534 on efferocytosis. To further assess the contribution of NAPE-PLD modulation on efferocytosis, BMDM was isolated from wild-type (WT) and Napepld^{-/-} (KO) mice and measured the extent of efferocytosis in the presence and absence of activator VU534. KO BMDM treated with vehicle (Veh) had significantly reduced efferocytosis compared to WT BMDM treated with Veh (FIG. 21C). In WT BMDM, treatment with activator VU534 significantly increased efferocytosis, but in KO BMDM, treatment with activator VU534 had no effect. Thus, NAPE-PLD appears to play a critical role in maximizing the efferocytosis capacity of macrophages, and activator VU534 enhances efferocytosis in a NAPE-PLD-dependent manner.

These studies demonstrate that select benzothiazole phenylsulfonyl-piperidine carboxamides significantly increase the activity of both mouse and human NAPE-PLD and that increasing NAPE-PLD activity increases efferocytosis by macrophages. The two most potent of the compound series tested, VU534 and VU533, incorporate a para-fluoro-group in the phenylsulfonyl moiety and di-methyl substitution of the benzothiazole aromatic ring. This preliminary SAR suggests that improved activators may be identified from a focused lead optimization campaign. Most of the tested analogs have minimal cytotoxicity against RAW264.7 and HepG2 cells, and their efficacy to enhance cellular NAPE-PLD activity correlates with their efficacy to enhance activity in the biochemical assay with purified recombinant NAPE-PLD. Although the lead series share structural features with some previously developed dual FAAH and sEH inhibitors, they show little inhibition of FAAH and only modest inhibition of sEH. Therefore, activators VU534 and VU533 should be useful tool compounds to assess the contribution of NAPE-PLD to various biological processes in cultured cells.

These studies provide further evidence that the NAPE-PLD/NAE signaling pathway plays a critical role in regulating efferocytosis. While NAPE-PLD expression was previously shown to be significantly reduced in unstable atherosclerotic plaques from human coronary arteries, and impaired efferocytosis has been implicated in the development of these plaques, these studies are the first to demonstrate that deletion of NAPE-PLD markedly diminished the ability of macrophages to carry out efferocytosis. Furthermore, inhibiting NAPE-PLD activity using bithionol phenocopied the effect of NAPE-PLD deletion. Consistent with the previous finding that treatment of BMDM with PEA enhances efferocytosis, treatment of wild-type BMDM with either VU534 or VU533 to increase NAPE-PLD activity also enhanced efferocytosis. This increase in efferocytosis required NAPE-PLD, as VU534 failed to enhance efferocytosis by Napepld^{-/-} BMDM.

Previously described effects of NAEs suggest some mechanisms by which increased NAPE-PLD activity could enhance efferocytosis. PEA acts via Gpr55 to increase the expression of MerTK, a receptor that helps macrophages recognize and bind to apoptotic cells. Deletion of MerTK in macrophages markedly enhances necrotic core expansion in Apoe^{-/-} mice. OEA also acts via PPARα to increase the expression of CD206 and TGFβ, two classic markers of the M2 macrophage phenotype with enhanced efferocytosis. A more complete elucidation of how NAPE-PLD regulates efferocytosis will require a variety of approaches including the use of both NAPE-PLD inhibitors and activators. Efferocytosis is a complex process involving macrophage recognition of so-called “find me” and “eat me” signals, and requires the binding, internalization, and controlled degradation of apoptotic cells, followed by export of their constituent components like cholesterol, so the effect of NAPE-PLD modulation on each of these steps needs to be examined. NAPEs exert membrane-stabilizing effects and facilitates the lateral diffusion of cholesterol, while phosphatidic acids exert membrane-bending effects. Therefore, the effect of increased NAPE-PLD activity on membrane topology as a mechanism to enhance efferocytosis also needs to be examined.

Although defective efferocytosis has been implicated in the progression to unstable atherosclerotic plaques, whether the enhanced efferocytosis induced by NAPE-PLD activators can translate to improved efferocytosis under atherogenic conditions requires future studies. Previous trials administering OEA, PEA, or NAE-boosting bacteria decrease the size of necrotic cores with atherosclerotic lesions. The poor pharmacokinetic properties of OEA and PEA have hampered their clinical use, while engineered bacteria that produce these bioactive lipids in situ still face significant regulatory hurdles for use for humans. While still at a very early stage, these results demonstrate that BT-PSP-based NAPE-PLD activators represent a potential alternative strategy to raise NAE levels and thereby achieve these same effects. It is worth noting that impaired efferocytosis has been implicated in a number of diseases besides atherosclerosis, including systemic lupus erythematosus, neurodegenerative diseases, retinal degeneration, pulmonary disorders, liver diseases, diabetes, inflammatory bowel disease, colon carcinoma, impaired wound healing, and rheumatoid arthritis. Therefore, future studies could also examine whether NAPE-PLD activators can protect against their development or progression. The value of NAPE-PLD activators as a therapeutic intervention may also extend beyond conditions with defective efferocytosis. For instance, NAPE-PLD expression and NAE levels are rapidly reduced by feeding a high-fat diet and administering OEA or PEA or their precursor NAPEs can markedly blunt the obesity, glucose intolerance, inflammation, and hepatosteatosis that results from these high-fat diets. Thus, future testing of NAPE-PLD activators should examine their potential to treat these conditions as well.

What is claimed:

1. A compound of formula (I): 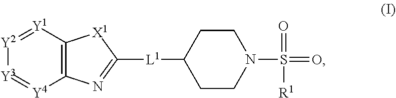
or a pharmaceutically acceptable salt thereof,
wherein:
X¹ is S or O;
Y¹ and Y³ are each C—R² and Y² and Y⁴ are each C—H, or
Y¹ and Y³ are each C—H and Y² and Y⁴ are each C—R²;
L¹ is 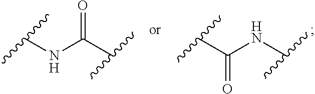 [SMILES: *NC(*)=O ; *NC(*)=O]
R¹ is G¹, —CH₂-G¹, or C₁₋₄alkyl;
R², at each occurrence, is independently C₁₋₄alkyl, C₁₋₄haloalkyl, halogen, C₃₋₄cycloalkyl, —OC₁₋₄alkyl, —OC₁₋₂haloalkyl, or —OC₃₋₄cycloalkyl;
G¹ is a phenyl, a 5- to 9-membered heteroaryl containing 1-3 heteroatoms, a 3- to 7-membered carbocycle, or a 4- to 6-membered heterocycle containing 1-2 heteroatoms, wherein G¹ is optionally substituted with 1 to 3 substituents, each independently R¹¹ or —C₁₋₂alkylene-R¹¹;
R¹¹ is C₁₋₄alkyl, C₁₋₄haloalkyl, C₃₋₄cycloalkyl, halogen, cyano, —OR^{11a}, —N(R^{11a})₂, —NR^{11a}C(O)R^{11b}, —C(O) R^{11b}, —CO₂R^{11b}, or —SO₂R^{11b};
R^{11a}, at each occurrence, are each independently hydrogen, C₁₋₄alkyl, C₁₋₄haloalkyl, or C₃₋₄cycloalkyl; and
R^{11b}, at each occurrence, is independently C₁₋₄alkyl, C₁₋₄haloalkyl, or C₃₋₄cycloalkyl;
with the proviso that the compound is not a compound of formula (I-a) 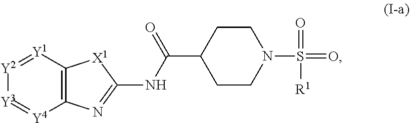
wherein:
i. X¹ is S,
Y¹ and Y³ are each C—CH₃, and Y² and Y⁴ are each C—H, or
Y¹ and Y³ are each C—H, and Y² and Y⁴ are each C—CH₃, Y² and Y⁴ are each C—F, or Y² is C—CI and Y⁴ is C—CH₃;
R¹ is methyl, 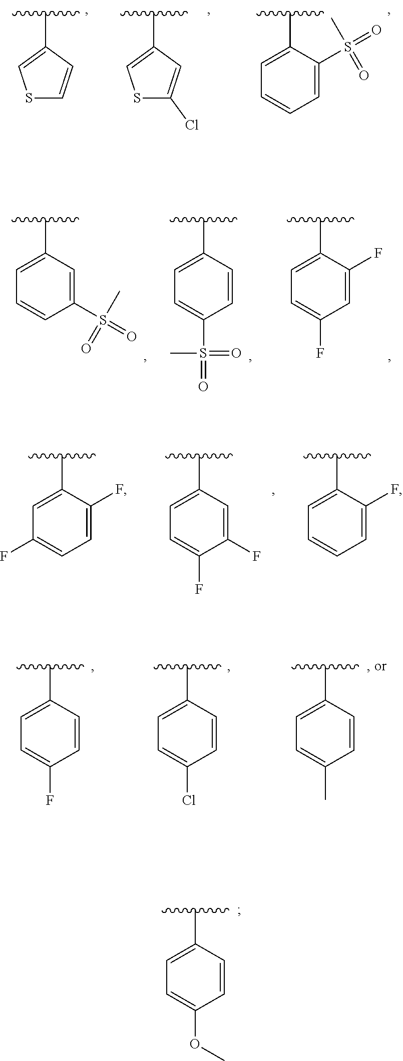
or wherein
ii. X¹ is S,
Y¹ and Y³ are each C—H, and Y² and Y⁴ are each C—CH₃, and
R¹ is ethyl, n-butyl, 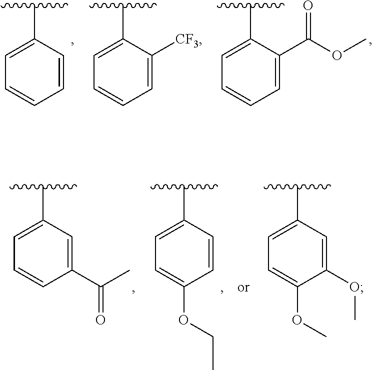
or wherein
iii. X¹ is S, Y¹ and Y³ are each C—H, Y² and Y⁴ are each C—F, and R¹ is 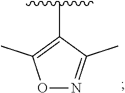
or wherein
iv. X¹ is O, Y¹ and Y³ are each C—CH₃, Y² and Y⁴ are each C—H, and R¹ is methyl.

2. The compound of claim 1, or a pharmaceutically acceptable salt thereof, wherein X¹ is S.

3. The compound of claim 1, or a pharmaceutically acceptable salt thereof, wherein Y¹ and Y³ are each C—C₁₋₄alkyl and Y² and Y⁴ are each C—H, or, where Y¹ and Y³ are each C—H and Y² and Y⁴ are each C—C₁₋₄alkyl.

4. The compound of claim 1, or a pharmaceutically acceptable salt thereof, wherein Y¹ and Y³ are each C—CH₃, and Y² and Y⁴ are each C—H.

5. The compound of claim 1, or a pharmaceutically acceptable salt thereof, wherein Y¹ and Y³ are each C—H, and Y² and Y⁴ are each C—CH₃.

6. The compound of claim 1, or a pharmaceutically acceptable salt thereof, wherein L¹ is

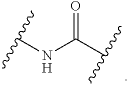 [SMILES: *NC(*)=O]

7. The compound of claim 1, or a pharmaceutically acceptable salt thereof, wherein R¹ is G¹.

8. The compound of claim 7, or a pharmaceutically acceptable salt thereof, wherein G¹ is the optionally substituted phenyl.

9. The compound of claim 8, or a pharmaceutically acceptable salt thereof, wherein G¹ is 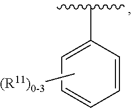
wherein R¹¹, at each occurrence, is independently halogen, cyano, C₁₋₄alkyl, C₁₋₂haloalkyl, —OC₁₋₄alkyl, or —OC₁₋₂haloalkyl.

10. The compound of claim 9, or a pharmaceutically acceptable salt thereof, wherein G¹ is

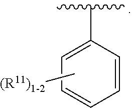

11. The compound of claim 10, or a pharmaceutically acceptable salt thereof, wherein G¹ is

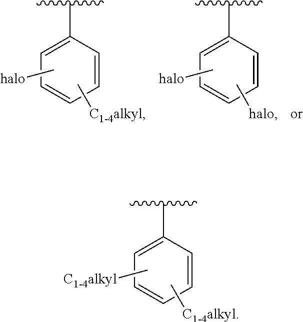

12. The compound of claim 11, or a pharmaceutically acceptable salt thereof, wherein G¹ is 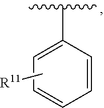
where R¹¹ is halogen, cyano, C₁₋₄alkyl, C₁₋₂haloalkyl, —OC₁₋₄alkyl, or —OC₁₋₂haloalkyl.

13. The compound of claim 7, or a pharmaceutically acceptable salt thereof, wherein G¹ is the optionally substituted 5- to 9-membered heteroaryl.

14. The compound of claim 13, or a pharmaceutically acceptable salt thereof, wherein the ring system of the optionally substituted 5- to 9-membered heteroaryl is a pyridinyl, a pyrimidinyl, a pyrazolyl, or a benzo[c][1,2,5]oxadiazolyl.

15. The compound of claim 14, or a pharmaceutically acceptable salt thereof, wherein R¹ is 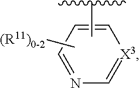
wherein X³ is C—H, or N, and R¹¹, at each occurrence, is independently halogen, cyano, C₁₋₄alkyl, C₁₋₂haloalkyl, —OC₁₋₄alkyl, or —OC₁₋₂haloalkyl.

16. The compound of claim 15, or a pharmaceutically acceptable salt thereof, wherein R¹ is

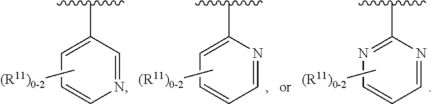

17. The compound of claim 16, or a pharmaceutically acceptable salt thereof, wherein R¹ is

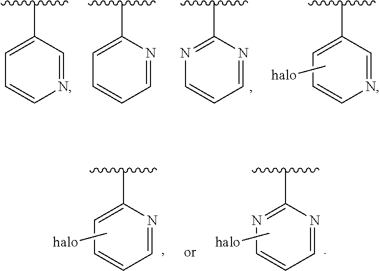

18. The compound of claim 1, or a pharmaceutically acceptable salt thereof, wherein R¹ is —CH₂-G¹.

19. The compound of claim 18, or a pharmaceutically acceptable salt thereof, wherein G¹ is the optionally substituted 3- to 7-membered carbocycle.

20. The compound of claim 19, or a pharmaceutically acceptable salt thereof, wherein the ring system of the optionally substituted 3- to 7-membered carbocycle is a 3- or 6-membered carbocycle.

21. The compound of claim 20, or a pharmaceutically acceptable salt thereof, wherein R¹ is

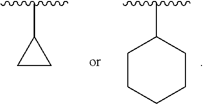

22. The compound of claim 1, or a pharmaceutically acceptable salt thereof, wherein R¹ is C₁₋₄alkyl.

23. The compound of claim 22, or a pharmaceutically acceptable salt thereof, wherein R¹ is methyl.

24. The compound of claim 1, wherein the compound is
1-((3-chloro-4-fluorophenyl)sulfonyl)-N-(5,7-dimethylbenzo[d]thiazol-2-yl)piperidine-4-carboxamide;
N-(4,6-dimethylbenzo[d]thiazol-2-yl)-1-((4-fluoro-2-methylphenyl)sulfonyl)piperidine-4-carboxamide;
N-(4,6-dimethylbenzo[d]thiazol-2-yl)-1-(pyridin-3-ylsulfonyl)piperidine-4-carboxamide;
N-(4,6-dimethylbenzo[d]thiazol-2-yl)-1-((1-methyl-1H-pyrazol-4-yl)sulfonyl)piperidine-4-carboxamide;
1-((2-chlorophenyl)sulfonyl)-N-(4,6-dimethylbenzo[d]thiazol-2-yl)piperidine-4-carboxamide;
N-(4,6-dimethylbenzo[d]thiazol-2-yl)-1-(o-tolylsulfonyl)piperidine-4-carboxamide;
N-(4,6-dimethylbenzo[d]thiazol-2-yl)-1-((3-fluorophenyl)sulfonyl)piperidine-4-carboxamide;
1-((3-chlorophenyl)sulfonyl)-N-(4,6-dimethylbenzo[d]thiazol-2-yl)piperidine-4-carboxamide;
N-(4,6-dimethylbenzo[d]thiazol-2-yl)-1-(m-tolylsulfonyl)piperidine-4-carboxamide;
1-((4-cyanophenyl)sulfonyl)-N-(4,6-dimethylbenzo[d]thiazol-2-yl)piperidine-4-carboxamide;
N-(4,6-dimethylbenzo[d]thiazol-2-yl)-1-((4-(trifluoromethoxy)phenyl)sulfonyl)piperidine-4-carboxamide;
1-((2-chloro-4-fluorophenyl)sulfonyl)-N-(4,6-dimethylbenzo[d]thiazol-2-yl)piperidine-4-carboxamide;
N-(4,6-dimethylbenzo[d]thiazol-2-yl)-1-((4-fluoro-2-methylphenyl)sulfonyl)piperidine-4-carboxamide;
N-(4,6-dimethylbenzo[d]thiazol-2-yl)-1-((6-fluoropyridin-3-yl)sulfonyl)piperidine-4-carboxamide;
N-(4,6-dimethylbenzo[d]thiazol-2-yl)-1-((5-fluoropyridin-2-yl)sulfonyl)piperidine-4-carboxamide;
N-(4,6-dimethylbenzo[d]thiazol-2-yl)-1-((5-fluoropyrimidin-2-yl)sulfonyl)piperidine-4-carboxamide;
N-(4,6-dimethylbenzo[d]thiazol-2-yl)-1-((2-fluoropyridin-3-yl)sulfonyl)piperidine-4-carboxamide;
N-(4,6-dimethylbenzo[d]thiazol-2-yl)-1-((3-fluoropyridin-2-yl)sulfonyl)piperidine-4-carboxamide;
N-(4,6-dimethylbenzo[d]thiazol-2-yl)-1-((3-fluoro-4-methylphenyl)sulfonyl)piperidine-4-carboxamide;
1-(cyclohexylsulfonyl)-N-(4,6-dimethylbenzo[d]thiazol-2-yl)piperidine-4-carboxamide;
1-((3,4-dichlorophenyl)sulfonyl)-N-(4,6-dimethylbenzo[d]thiazol-2-yl)piperidine-4-carboxamide;
1-((2,4-difluorobenzyl)sulfonyl)-N-(4,6-dimethylbenzo[d]thiazol-2-yl)piperidine-4-carboxamide;
1-(cyclopropylsulfonyl)-N-(4,6-dimethylbenzo[d]thiazol-2-yl)piperidine-4-carboxamide;
1-((cyclopropylmethyl)sulfonyl)-N-(4,6-dimethylbenzo[d]thiazol-2-yl)piperidine-4-carboxamide;
1-(benzo[c][1,2,5]oxadiazol-4-ylsulfonyl)-N-(4,6-dimethylbenzo[d]thiazol-2-yl)piperidine-4-carboxamide;
1-((3-chloro-4-fluorophenyl)sulfonyl)-N-(5,7-dimethylbenzo[d]thiazol-2-yl)piperidine-4-carboxamide;
N-(5,7-dimethylbenzo[d]thiazol-2-yl)-1-((4-fluoro-2-methylphenyl)sulfonyl)piperidine-4-carboxamide;
N-(5,7-dimethylbenzo[d]thiazol-2-yl)-1-(pyridin-3-ylsulfonyl)piperidine-4-carboxamide;
N-(5,7-dimethylbenzo[d]thiazol-2-yl)-1-((1-methyl-1H-pyrazol-4-yl)sulfonyl)piperidine-4-carboxamide;
1-((2-chlorophenyl)sulfonyl)-N-(5,7-dimethylbenzo[d]thiazol-2-yl)piperidine-4-carboxamide;
N-(5,7-dimethylbenzo[d]thiazol-2-yl)-1-(o-tolylsulfonyl)piperidine-4-carboxamide;
N-(5,7-dimethylbenzo[d]thiazol-2-yl)-1-((3-fluorophenyl)sulfonyl)piperidine-4-carboxamide;
1-((3-chlorophenyl)sulfonyl)-N-(5,7-dimethylbenzo[d]thiazol-2-yl)piperidine-4-carboxamide;
N-(5,7-dimethylbenzo[d]thiazol-2-yl)-1-(m-tolylsulfonyl)piperidine-4-carboxamide;
1-((4-cyanophenyl)sulfonyl)-N-(5,7-dimethylbenzo[d]thiazol-2-yl)piperidine-4-carboxamide;
N-(5,7-dimethylbenzo[d]thiazol-2-yl)-1-((4-(trifluoromethoxy)phenyl)sulfonyl)piperidine-4-carboxamide;
1-((2-chloro-4-fluorophenyl)sulfonyl)-N-(5,7-dimethylbenzo[d]thiazol-2-yl)piperidine-4-carboxamide;
N-(5,7-dimethylbenzo[d]thiazol-2-yl)-1-((4-fluoro-2-methylphenyl)sulfonyl)piperidine-4-carboxamide;
N-(5,7-dimethylbenzo[d]thiazol-2-yl)-1-((6-fluoropyridin-3-yl)sulfonyl)piperidine-4-carboxamide;
N-(5,7-dimethylbenzo[d]thiazol-2-yl)-1-((5-fluoropyridin-2-yl)sulfonyl)piperidine-4-carboxamide;
N-(5,7-dimethylbenzo[d]thiazol-2-yl)-1-((5-fluoropyrimidin-2-yl)sulfonyl)piperidine-4-carboxamide;
N-(5,7-dimethylbenzo[d]thiazol-2-yl)-1-((2-fluoropyridin-3-yl)sulfonyl)piperidine-4-carboxamide;
N-(5,7-dimethylbenzo[d]thiazol-2-yl)-1-((3-fluoropyridin-2-yl)sulfonyl)piperidine-4-carboxamide;
N-(5,7-dimethylbenzo[d]thiazol-2-yl)-1-((3-fluoro-4-methylphenyl)sulfonyl)piperidine-4-carboxamide;
1-(cyclohexylsulfonyl)-N-(5,7-dimethylbenzo[d]thiazol-2-yl)piperidine-4-carboxamide;
1-((3,4-dichlorophenyl)sulfonyl)-N-(5,7-dimethylbenzo[d]thiazol-2-yl)piperidine-4-carboxamide;
1-((2,4-difluorobenzyl)sulfonyl)-N-(5,7-dimethylbenzo[d]thiazol-2-yl)piperidine-4-carboxamide;
1-(cyclopropylsulfonyl)-N-(5,7-dimethylbenzo[d]thiazol-2-yl)piperidine-4-carboxamide;
1-((cyclopropylmethyl)sulfonyl)-N-(5,7-dimethylbenzo[d]thiazol-2-yl)piperidine-4-carboxamide; or
1-(benzo[c][1,2,5]oxadiazol-4-ylsulfonyl)-N-(5,7-dimethylbenzo[d]thiazol-2-yl)piperidine-4-carboxamide.

25. A pharmaceutical composition comprising the compound of claim 1 and a pharmaceutically acceptable carrier.

26. A pharmaceutical composition comprising a compound of formula (I) 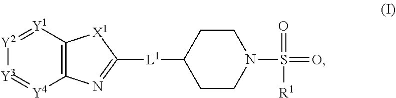
or a pharmaceutically acceptable salt thereof,
wherein:
X¹ is S or O;
Y¹, Y², Y³, and Y⁴ are each C—R² or C—H;
L¹ is 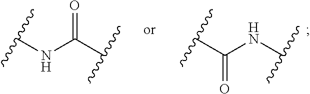 [SMILES: *NC(*)=O ; *NC(*)=O]
R¹ is G¹, —CH₂-G¹, or C₁₋₄alkyl;
R², at each occurrence, is independently C₁₋₄alkyl, C₁₋₄haloalkyl, halogen, C₃₋₄cycloalkyl, —OC₁₋₄alkyl, —OC₁₋₂haloalkyl, or —OC₃₋₄cycloalkyl;
G¹ is a phenyl, a 5- to 9-membered heteroaryl containing 1-3 heteroatoms, a 3- to 7-membered carbocycle, or a 4- to 6-membered heterocycle containing 1-2 heteroatoms, wherein G¹ is optionally substituted with 1 to 3 substituents, each independently R¹¹ or —C₁₋₂alkylene-R¹¹;
R¹¹ is C₁₋₄alkyl, C₁₋₄haloalkyl, C₃₋₄cycloalkyl, halogen, cyano, —OR^{11a}, —N(R^{11a})₂, —NR^{11a}C(O)R^{11b}, —C(O) R^{11b}, —CO₂R^{11b}, or —SO₂R^{11b};
R^{11a}, at each occurrence, are each independently hydrogen, C₁₋₄alkyl, C₁₋₄haloalkyl, or C₃₋₄cycloalkyl; and
R^{11b}, at each occurrence, is independently C₁₋₄alkyl, C₁₋₄haloalkyl, or C₃₋₄cycloalkyl;
and a pharmaceutically acceptable carrier,
with the proviso that the compound is not a compound of formula (I-b) 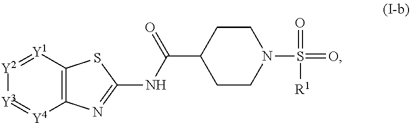
wherein:
i. Y¹, Y³, and Y⁴ are each C—H, Y² is C—CH₃, C—OCH₃, or C—OCH₂CH₃, and R¹ is 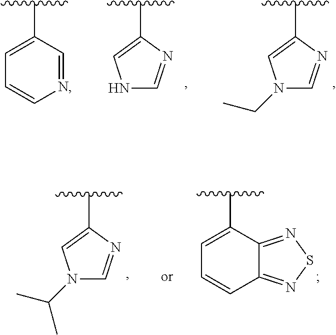
or wherein
ii. Y¹, Y³, and Y⁴ are each C—H, Y² is C—H, C—CH₃, C—OCH₃, or C—F, and R¹ is 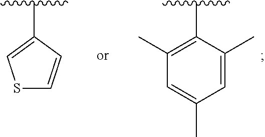
or wherein
iv. Y¹, Y³, and Y⁴ are each C—H, Y⁴ is C—CH₃, and R¹ is 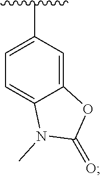 [SMILES: *c1ccc2c(c1)oc(=O)n2C]
or wherein
v. Y¹ and Y⁴ are each C—H, Y² and Y³ are each C—OH, and R¹ is

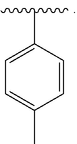 [SMILES: *c1ccc(C)cc1]

27. A method for treating a disease or disorder associated with metabolic dysfunction in a mammal, the method comprising administering to the mammal a therapeutically effective amount of the compound of claim 1, or pharmaceutically acceptable salt thereof, or the pharmaceutical composition of claim 26.

28. The method of claim 27, wherein the disease or disorder is associated with N-acyl phosphatidylethanolamine hydrolyzing phospholipase D (NAPE-PLD) dysfunction.

29. The method of claim 27, wherein the disease or disorder is obesity, type 2 diabetes, hyperlipidemia, non-alcoholic fatty liver disease, atherosclerosis, hypertriglyceridemia, or hypertension.

30. The method of claim 27, wherein the disease or disorder is a non-healing wound, a chronic ulcer of the leg or foot, cellulitis or abscess of the leg, or gangrene.

31. The pharmaceutical composition of claim 26, wherein the pharmaceutical composition is formulated for topical administration.

32. The compound of claim 1, or pharmaceutically acceptable salt thereof, or the pharmaceutical composition of claim 26, for use in the treatment of a disease or disorder associated with metabolic dysfunction in a mammal.

33. The use of the compound of claim 1, or pharmaceutically acceptable salt thereof, or the pharmaceutical composition of claim 26, for the preparation of a medicament for the treatment of a disease or disorder associated with metabolic dysfunction in a mammal.
